(19) United States
(12) Patent Application Publication
(10) Pub. No.: US 2007/0270311 A1
(43) Pub. Date: Nov. 22, 2007

(54) 6-Phenyl-7-Aminotriazolopyrimidines, Processes For Their Preparation And Their Use For Controlling Harmful Fungi, And Also Compositions Comprising Them

(75) Inventors: Carsten Blettner, Hong Kong (CN); Jordi Tormo i Blasco, Laudenbach (DE); Bernd Muller, Frankenthal (DE); Markus Gewehr, Kastellaun (DE); Wassilios Grammenos, Ludwigshafen (DE); Thomas Grote, Wachenheim (DE); Udo Hunger, Mainz (DE); Joachim Rheinheimer, Ludwigshafen (DE); Peter Schafer, Ottersheim (DE); Frank Schieweck, Hessheim (DE); Anja Schwogler, Mannheim (DE); Jochen Dietz, Mannheim (DE); John-Bryan Speakman, Bobenheim (DE); Thorsten Jabs, Hassloch (DE); Siegfried Strathmann, Limburgerhof (DE); Ulrich Schofl, Bruhl (DE); Maria Scherer, Landau (DE); Reinhard Stierl, Freinsheim (DE)

Correspondence Address: HUTCHISON LAW GROUP PLLC, PO BOX 31686, RALEIGH, NC, 27612, US

(73) Assignee: BASF AKTIENGESELLSCHAFT, Ludwigshafen (DE)

(21) Appl. No.: 11/661,566

(22) PCT Filed: Sep. 2, 2005
(86) PCT No.: PCT/EP05/09456

§ 371 Date: Feb. 28, 2007

(30) Foreign Application Priority Data
Sep. 8, 2004 (DE) 10 2004 043 836.6

(51) Int. Cl. A01N 43/90; A01P 3/00; C07D 487/02

(52) U.S. Cl. 504/100; 514/259.31; 544/263

(57) ABSTRACT

6-Phenyl-7-aminotriazolopyrimidines of the formula I 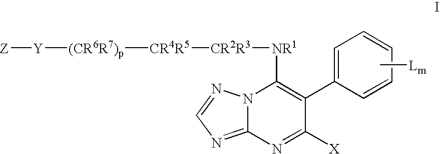 in which the substituents are as defined below:
R¹ is hydrogen, alkyl, haloalkyl, cycloalkyl, halocycloalkyl, alkenyl, haloalkenyl, cycloalkenyl, halocycloalkenyl, alkynyl, haloalkynyl or phenyl, naphthyl, or a five- or six-membered saturated, partially unsaturated or aromatic heterocycle, which comprises one to four heteroatoms from the group consisting of O, N and S,
R² is alkyl, haloalkyl, cycloalkyl, halocycloalkyl, alkenyl, haloalkenyl, cycloalkenyl, halocycloalkenyl, alkynyl, haloalkynyl or phenyl, naphthyl, or a five- or six-membered saturated, partially unsaturated or aromatic heterocycle which comprises one to four heteroatoms from the group consisting of O, N and S,
R³,R⁴,R⁵,R⁶,R⁷ are hydrogen or one of the groups mentioned under R², R⁴, together with R³ or R⁶, may also form a five- or six-membered saturated or unsaturated ring which, in addition to carbon atoms, may comprise one to three further heteroatoms from the group consisting of O, N and S as ring members; R² with R³, R⁴ with R⁵, R⁶ with R⁷ may in each case together, with formation of spiro groups, also form a C₂-C₅-alkylene, or alkenylene or alkynylene chain which may be interrupted by one to three heteroatoms from the group consisting of O, N and S; p is zero or 1;
L is halogen, alkyl, haloalkyl, alkoxy, cyano, nitro, amino, alkylamino, dialkylamino, alkylcarbonylamino, C(O)—R, S(O)ₙ—R; where n is zero, 1 or 2; R is hydrogen, alkyl, haloalkyl, alkoxy, alkenyloxy, alkynyloxy, amino, alkylamino, dialkylamino; m is 1, 2, 3, 4 or 5; X is halogen, cyano, alkyl, alkoxy, alkenyloxy, alkynyloxy or haloalkoxy; Y is oxygen or sulfur; Z is hydrogen, alkyl, haloalkyl, cycloalkyl, alkylcarbonyl, cycloalkylcarbonyl, alkenyl, haloalkenyl, cycloalkenyl, alkynyl, haloalkynyl, phenyl, naphthyl, a five- to ten-membered saturated, partially unsaturated or aromatic heterocycle which comprises one to four heteroatoms from the group consisting of O, N and S; or
   Z together with R⁴ or R⁶ may also form a five- or six-membered saturated or unsaturated ring which, in addition to carbon atoms and Y, may comprise one or two further heteroatoms from the group consisting of O, N and S as ring members the groups R¹ to R⁷, Z and R may be substituted according to the description; processes for preparing these compounds, compositions comprising them and their use for controlling phytopathogenic harmful fungi.

The present invention relates to 6-phenyl-7-aminotriazolopyrimidines of the formula I 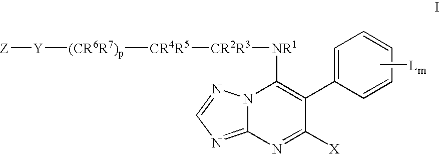
in which the substituents are as defined below:
R¹ is hydrogen, C₁-C₁₂-alkyl, C₁-C₁₂-haloalkyl, C₃-C₆-cycloalkyl, C₃-C₈-halocycloalkyl, C₂-C₁₂-alkenyl, C₂-C₁₂-haloalkenyl, C₃-C₆-cycloalkenyl, C₃-C₆-halocycloalkenyl, C₂-C₁₂-alkynyl, C₂-C₁₂-haloalkynyl or phenyl, naphthyl, or a five- or six-membered saturated, partially unsaturated or aromatic heterocycle, which comprises one to four heteroatoms from the group consisting of O, N and S,
R² is C₁-C₈-alkyl, C₁-C₆-haloalkyl, C₃-C₆-cycloalkyl, C₃-C₆-halocycloalkyl, C₂-C₈-alkenyl, C₂-C₈-haloalkenyl, C₃-C₆-cycloalkenyl, C₃-C₆-halocycloalkenyl, C₂-C₈-alkynyl, C₂-C₈-haloalkynyl or phenyl, naphthyl, or a five- or six-membered saturated, partially unsaturated or aromatic heterocycle which comprises one to four heteroatoms from the group consisting of O, N and S,
R³,R⁴,R⁵,R⁶,R⁷ are hydrogen or one of the groups mentioned under R²,
   R⁴, together with R³ or R⁶, may also form a five- or six-membered saturated or unsaturated ring which, in addition to carbon atoms, may comprise one to three further heteroatoms from the group consisting of O, N and S as ring members and/or may carry one or more substituents R^{a};
   R² with R³, R⁴ with R⁵, R⁶ with R⁷ may in each case together, with formation of carbonyl groups, also be oxygen and, with formation of spiro groups, form a C₂-C₅-alkylene, or alkenylene or alkynylene chain which may be interrupted by one to three heteroatoms from the group consisting of O, N and S;
R¹ to R⁷ may in each case carry one to four identical or different groups R^{a}:
   R^{a} is halogen, cyano, nitro, hydroxyl, C₁-C₆-alkyl, C₁-C₆-haloalkyl, C₁-C₆-alkylcarbonyl, C₃-C₆-cycloalkyl, C₁-C₆-alkoxy, C₁-C₆-haloalkoxy, C₁-C₆-alkoxycarbonyl, C₁-C₆-alkylthio, C₁-C₆-alkylamino, di-C₁-C₆-alkyl-amino, C₂-C₈-alkenyl, C₂-C₈-haloalkenyl, C₃-C₈-cycloalkenyl, C₂-C₆-alkenyloxy, C₃-C₆-haloalkenyloxy, C₂-C₆-alkynyl, C₂-C₆-haloalkynyl, C₃-C₆-alkynyloxy, C₃-C₆-haloalkynyloxy, C₃-C₆-cycloalkoxy, C₃-C₆-cycloalkenyloxy, oxy-C₁-C₃-alkyleneoxy, phenyl, naphthyl, a five- to ten-membered saturated, partially unsaturated or aromatic heterocycle which comprises one to four heteroatoms from the group consisting of O, N and S,
   where these aliphatic, alicylic or aromatic groups for their part may be partially or fully halogenated or may carry one to three groups R^{b}:
   R^{b} is halogen, cyano, nitro, hydroxyl, mercapto, amino, carboxyl, aminocarbonyl, aminothiocarbonyl, alkyl, haloalkyl, alkenyl, alkenyloxy, alkynyloxy, alkoxy, haloalkoxy, alkylthio, alkylamino, dialkylamino, formyl, alkylcarbonyl, alkylsulfonyl, alkylsulfoxyl, alkoxycarbonyl, alkylcarbonyloxy, alkylaminocarbonyl, dialkylaminocarbonyl, alkylaminothiocarbonyl, dialkylaminothiocarbonyl, where the alkyl groups in these radicals comprise 1 to 6 carbon atoms and the abovementioned alkenyl or alkynyl groups in these radicals comprise 2 to 8 carbon atoms;
      and/or one to three of the following radicals:
      cycloalkyl, cycloalkoxy, heterocyclyl, heterocyclyloxy, where the cyclic systems comprise 3 to 10 ring members; aryl, aryloxy, arylthio, aryl-C₁-C₆-alkoxy, aryl-C₁-C₆-alkyl, hetaryl, hetaryloxy, hetarylthio, where the aryl radicals preferably comprise 6 to 10 ring members and the hetaryl radicals 5 or 6 ring members, where the cyclic systems may be partially or fully halogenated or substituted by alkyl or haloalkyl groups;
p is zero or 1;
L is halogen, C₁-C₄-alkyl, C₁-C₂-haloalkyl, C₁-C₄-alkoxy, cyano, nitro, amino, C₁-C₄-alkylamino, di-(C₁-C₄)-alkylamino, C₁-C₄-alkylcarbonylamino, C(O)—R, S(O)ₙ—R;
   R is hydrogen, C₁-C₄-alkyl, C₁-C₂-haloalkyl, C₁-C₄-alkoxy, C₂-C₄-alkenyloxy, C₂-C₄-alkynyloxy, amino, C₁-C₄-alkylamino, di-(C₁-C₄)-alkylamino; where the groups R may be substituted by one to three groups R^{b};
   n is zero, 1 or 2;
m is 1, 2, 3, 4 or 5;
X is halogen, cyano, C₁-C₄-alkyl, C₁-C₄-alkoxy, C₂-C₄-alkenyloxy, C₂-C₄-alkynyloxy or C₁-C₂-haloalkoxy,
Y is oxygen or sulfur;
Z is hydrogen, C₁-C₈-alkyl, C₁-C₆-haloalkyl, C₃-C₆-cycloalkyl, C₁-C₈-alkylcarbonyl, C₁-C₈-alkoxycarbonyl, (C═O)NR^{A}R^{B}, C₃-C₈-alkenyloxycarbonyl, C₃-C₈-alkynyloxycarbonyl, C₃-C₆-cycloalkylcarbonyl, C₂-C₈-alkenyl, C₂-C₈-haloalkenyl, C₃-C₈-cycloalkenyl, C₂-C₆-alkynyl, C₂-C₆-haloalkynyl, C₃-C₆-cycloalkoxycarbonyl, C₃-C₆-cycloalkenyloxycarbonyl, C₁-C₈-alkylsulfinyl, C₁-C₈-alkylthio, C₁-C₈-alkylsulfonyl, phenyl, naphthyl, a five- to ten-membered saturated, partially unsaturated or aromatic heterocycle which comprises one to four heteroatoms from the group consisting of O, N and S; or
   Z together with R⁵ or R⁷ may also form a five- or six-membered saturated or unsaturated ring which, in addition to carbon atoms and Y, may comprise one or two further heteroatoms from the group consisting of O, N and S as ring members and/or may carry one or more substituents R^{a};
   the group Z may be partially or fully halogenated or carry one to three groups R^{b};
   R^{A}, R^{B} are independently hydrogen, C₁-C₈-alkyl, C₂-C₈-alkenyl, C₂-C₈-alkynyl, C₃-C₆-cycloalkyl or C₃-C₆-cycloalkenyl;
      R^{A} and R^{B} may combine with the nitrogen atom to which they are attached to form a five- or six-membered saturated, partially unsaturated or aromatic ring which, as well as carbon atoms, may comprise from one to three further heteroatoms from the group consisting of O, N and S as a ring member and/or may carry one or more substituents R^{a}.

Moreover, the invention relates to processes and intermediates for preparing these compounds, to compositions comprising them and to their use for controlling phytopathogenic harmful fungi.

6-Phenyl-7-aminotriazolopyrimidines are known in a general manner from EP-A 71 792. 6-Phenyl-7-aminotriazolopyrimidines having 7-amino substituents comprising hydroxyl or ether groups are disclosed in EP-A 550 113, U.S. Pat. No. 5,993,996, U.S. Pat. No. 6,117,865, U.S. Pat. No. 6,297,251 and WO 98/46607. These compounds are known to be suitable for controlling harmful fungi.

The compounds according to the invention differ from the compounds disclosed in the abovementioned applications by the specific embodiment of the 7-amino group, which is branched at the α-carbon atom.

In many cases, the activity of the known compounds is unsatisfactory. Based on this, it is an object of the present invention to provide compounds having improved activity and/or a broader activity spectrum.

We have found that this is achieved by the compounds defined at the outset. Furthermore, we have found processes and intermediates for their preparation, compositions comprising them and methods for controlling harmful fungi using the compounds I.

The compounds according to the invention can be obtained by different routes. Advantageously, they are prepared by reacting 7-dihalotriazolopyrimidines of the formula II with amines of the formula III in which the variables are as defined for formula I.

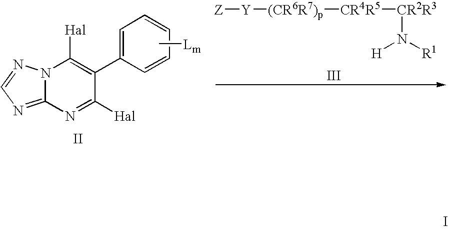

This reaction is advantageously carried out at from 0° C. to 70° C., preferably from 10° C. to 35° C., preferably in the presence of an inert solvent, such as an ether, for example, dioxane, diethyl ether or, in particular, tetrahydrofuran, a halogenated hydrocarbon, such as dichloromethane, or an aromatic hydrocarbon, such as, for example, toluene [cf. WO-A 98/46608].

Preference is given to using a base, such as a tertiary amine, for example triethylamine or an inorganic amine, such as potassium carbonate; it is also possible for excess amine of the formula III to serve as base.

By employing the 5,7-dihalotriazolopyrimidines known from EP-A 550 113 and EP-A 770 615, it is thus possible to access the 5-halotriazolopyrimidines of the formula I in which X is halogen, preferably chlorine. They form a preferred subject matter of the invention. Other 5,7-dihalotriazolopyrimidines are accessible analogously to the literature cited.

Depending on the embodiment of the group Z in formula I, it may be advantageous to react II with an appropriate hydroxyl- or mercaptoamine (Z=hydrogen) of the formula IIIa and to introduce the group Z at the stage of the 7-hydroxy or -mercapto-aminotriazolopyrimidine of the formula Ia formed, by etherification or esterification.

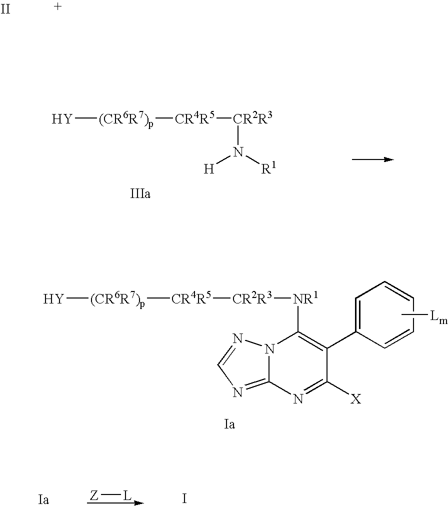

To this end, the 7-hydroxy or -mercaptoaminotriazolopyrimidine of the formula Ia is reacted with an alkylating or acylating agent Z-L, where L is a nucleophilically replaceable group. The person skilled in the art is generally familiar with the reaction conditions suitable for the etherification or esterification [cf.: Organikum, VEB Deutscher Verlag der Wissenschaften, Berlin (1981)].

Amines of the formulae III and IIIa are known from the literature, can be prepared by known methods or are commercially available.

Compounds of the formula I in which X is cyano, C₁-C₆-alkoxy or C₁-C₂-haloalkoxy can be obtained in an advantageous manner by reacting compounds I, in which X is halogen, preferably chlorine, with compounds M-X′ (formula IV). Depending on the meaning of the group X′ to be introduced, the compounds IV are inorganic cyamides, alkoxides or haloalkoxides. The reaction is advantageously carried out in the presence of an inert solvent. The cation M in formula IV is of little importance; for practical reasons, preference is usually given to ammonium, tetraalkylammonium or alkali metal or alkaline earth metal salts.
I(X=halogen)+M-X′→(X═X′) IV

The reaction temperature is usually from 0 to 120° C., preferably from 10 to 40° C. [cf. J. Heterocycl. Chem., 12, (1975), 861-863].

Suitable solvents including ethers, such as dioxane, diethyl ether and, preferably, tetrahydrofuran, halogenated hydrocarbons, such as dichloromethane and aromatic hydrocarbons, such as toluene.

Compounds of the formula I in which X is C₁-C₄-alkyl can be obtained in an advantageous manner via the following synthesis route:

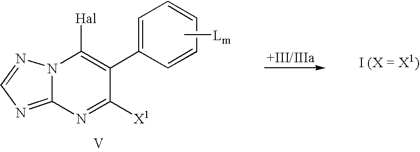

The reaction of the 5-alkyl-7-halotriazolopyrimidines of the formula V in which X¹ is C₁-C₄-alkyl or C₁-C₄-haloalkyl with amines III or IIIa is carried out under the conditions described further above. Compounds of the formula V are known from WO 03/093271 or can be prepared in accordance with the literature cited.

Alternatively, compounds of the formula I, in which X is C₁-C₄-alkyl can also be prepared from compounds I in which X is in particular chlorine and malonates of the formula VI. In formula VI, X″ is hydrogen or C₁-C₃-alkyl and R is C₁-C₄-alkyl. They are converted into compounds of the formula VII and decarboxylated to give compounds I [cf. U.S. Pat. No. 5,994,360].

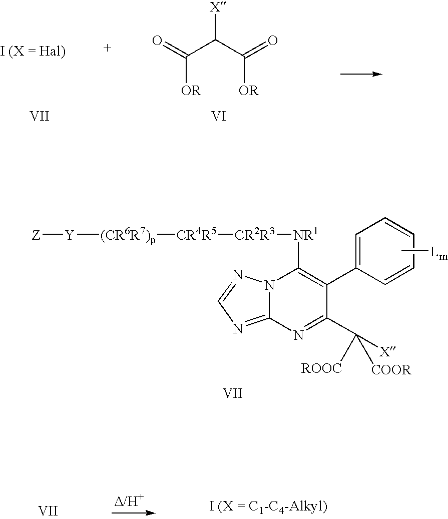

The malonates VI are known from the literature [J. Am. Chem. Soc., 64, (1942), 2714; J. Org. Chem., 39, (1974), 2172; Helv. Chim. Acta, 61, (1978), 1565], or they can be prepared in accordance with the literature cited.

The subsequent hydrolysis of the ester VII is carried out under generally customary conditions; depending on the various structural elements, alkali or acidic hydrolysis of the compounds VII may be advantageous. Under the conditions of ester hydrolysis, there may already be complete or partial decarboxylation to 1.

The decarboxylation is usually carried out at temperatures of from 20° C. to 180° C., preferably from 50° C. to 120° C., in an inert solvent, if appropriate in the presence of an acid.

Suitable acids are hydrochloric acid, sulfuric acid, phosphoric acid, formic acid, acetic acid, p-toluenesulfonic acid. Suitable solvents are water, aliphatic hydrocarbons, such as pentane, hexane, cyclohexane and petroleum ether, aromatic hydrocarbons, such as toluene, o-, m- and p-xylolene, halogenated hydrocarbons, such as methylene chloride, chloroform and chlorobenzene, ethers, such as diethyl ether, diisopropyl ether, tert-butyl methyl ether, dioxane, anisole and tetrahydrofuran, nitriles, such as acetonitrile and propionitrile, ketones, such as acetone, methyl ethyl ketone, diethyl ketone and tert-butyl methyl ketone, alcohols, such as methanol, ethanol, n-propanol, isopropanol, n-butanol and tert-butanol, and also dimethyl sulfoxide, dimethyl-formamide and dimethylacetamide; with particular preference, the reaction is carried out in hydrochloric acid or acetic acid. It is also possible to use mixtures of the solvents mentioned.

Compounds of the formula I in which X is C₁-C₄-alkyl can also be obtained by coupling 5-halotriazolopyrimidines of the formula I with organometallic reagents of the formula VIII. In one embodiment of this process, the reaction is carried out with transition metal catalysis, such as Ni- or Pd catalysis.
I(X=Hal)+M^{y}(—X″)_{y}→I(X═C₁-C₄-alkyl) VIII

In formula VIII, M is a metal ion of valency Y, such as, for example, B, Zn or Sn, and X″ is C₁-C₃-alkyl. This reaction can be carried out, for example, analogously to the following methods: J. Chem. Soc. Perkin Trans. 1 (1994), 1187, ibid. 1 (1996), 2345; WO 99/41255; Aust. J. Chem., 43(1990), 733; J. Org. Chem., 43 (1978), 358; J. Chem. Soc. Chem. Commun. (1979), 866; Tetrahedron Lett., 34 (1993), 8267; ibid. 33 (1992), 413.

The reaction mixtures are worked up in a customary manner, for example by mixing with water, separating the phases and, if appropriate, chromatographic purification of the crude products. Some of the intermediates and end products are obtained in the form of colorless or slightly brownish viscous oils which can be purified or freed from volatile components under reduced pressure and at moderately elevated temperature. If the intermediates and end products are obtained as solids, purification can also be carried out by recrystallization or digestion.

If individual compounds I cannot be obtained by the routes described above, they can be prepared by derivatization of other compounds I.

If the synthesis yields mixtures of isomers, a separation is generally not necessarily required since in some cases the individual isomers can be interconverted during work-up for use or during application (for example under the action of light, acids or bases). Such conversions may also take place after use, for example in the treatment of plants, in the treated plant, or in the harmful fungus to be controlled.

In the definitions of the symbols given in the formulae above, collective terms were used which are generally representative of the following substituents: halogen: fluorine, chlorine, bromine and iodine;

alkyl: saturated straight-chain or branched hydrocarbon radicals having 1 to 4, 6 or 8 carbon atoms, for example C₁-C₆-alkyl such as methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl;

haloalkyl: straight-chain or branched alkyl groups having 1 to 2, 4 or 6 carbon atoms (as mentioned above), where in these groups some or all of the hydrogen atoms may be replaced by halogen atoms as mentioned above; in particular, C₁-C₂-haloalkyl, such as chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl or 1,1,1-trifluoroprop-2-yl;

alkenyl: unsaturated straight-chain or branched hydrocarbon radicals having 2 to 4, 6 or 8 carbon atoms and one or two double bonds in any position, for example C₂-C₆-alkenyl, such as ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-penienyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl;

haloalkenyl: unsaturated straight-chain or branched hydrocarbon radicals having 2 to 8 carbon atoms and one or two double bonds in any position (as mentioned above), where in these groups some or all of the hydrogen atoms may be replaced by halogen atoms as mentioned above, in particular by fluorine, chlorine and bromine;

alkynyl: straight-chain or branched hydrocarbon groups having 2 to 4, 6 or 8 carbon atoms and one or two triple bonds in any position, for example C₂-C₆-alkynyl, such as ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl and 1-ethyl-1-methyl-2-propynyl;

cycloalkyl: mono- or bicyclic saturated hydrocarbon groups having 3 to 6 or 8 carbon ring members, for example C₃-C₈-cycloalkyl such as cyclopropyl, cyclobutyl, cyclopentyl, cyclohexyl, cycloheptyl and cyclooctyl;

five- to ten-membered saturated, partially unsaturated or aromatic heterocycle which comprises one to four heteroatoms from the group consisting of O, N and S:

5- or 6-membered heterocyclyl which contains one to three nitrogen atoms and/or one oxygen or sulfur atom or one or two oxygen and/or sulfur atoms, for example 2-tetrahydrofuranyl, 3-tetrahydrofuranyl, 2-tetrahydrothienyl, 3-tetrahydrothienyl, 2-pyrrolidinyl, 3-pyrrolidinyl, 3-isoxazolidinyl, 4-isoxazolidinyl, 5-isoxazolidinyl, 3-isothiazolidinyl, 4-isothiazolidinyl, 5-isothiazolidinyl, 3-pyrazolidinyl, 4-pyrazolidinyl, 5-pyrazolidinyl, 2-oxazolidinyl, 4-oxazolidinyl, 5-oxazolidinyl, 2-thiazolidinyl, 4-thiazolidinyl, 5-thiazolidinyl, 2-imidazolidinyl, 4-imidazolidinyl, 2-pyrrolin-2-yl, 2-pyrrolin-3-yl, 3-pyrrolin-2-yl, 3-pyrrolin-3-yl, 2-piperidinyl, 3-piperidinyl, 4-piperidinyl, 1,3-dioxan-5-yl, 2-tetrahydropyranyl, 4-tetrahydropyranyl, 2-tetrahydrothienyl, 3-hexahydropyridazinyl, 4-hexahydropyridazinyl, 2-hexahydropyrimidinyl, 4-hexahydropyrimidinyl, 5-hexahydropyrimidinyl and 2-piperazinyl;
5-membered heteroaryl which comprises one to four nitrogen atoms or one to three nitrogen atoms and one sulfur or oxygen atom: 5-membered heteroaryl groups which, in addition to carbon atoms, may comprise one to four nitrogen atoms or one to three nitrogen atoms and one sulfur or oxygen atom as ring members, for example 2-furyl, 3-furyl, 2-thienyl, 3-thienyl, 2-pyrrolyl, 3-pyrrolyl, 3-pyrazolyl, 4-pyrazolyl, 5-pyrazolyl, 2-oxazolyl, 4-oxazolyl, 5-oxazolyl, 2-thiazolyl, 4-thiazolyl, 5-thiazolyl, 2-imidazolyl, 4-imidazolyl and 1,3,4-triazol-2-yl;
6-membered heteroaryl which comprises one to three or one to four nitrogen atoms: 6-membered heteroaryl groups which, in addition to carbon atoms, may contain one to three or one to four nitrogen atoms as ring members, for example 2-pyridinyl, 3-pyridinyl, 4-pyridinyl, 3-pyridazinyl, 4-pyridazinyl, 2-pyrimidinyl, 4-pyrimidinyl, 5-pyrimidinyl and 2-pyrazinyl;
   alkylene: divalent unbranched chains of 3 to 5 CH₂ groups, for example CH₂, CH₂CH₂, CH₂CH₂CH₂, CH₂CH₂CH₂CH₂ and CH₂CH₂CH₂CH₂CH₂;
   oxyalkylene: divalent unbranched chains of 2 to 4 CH₂ groups, where one valency is attached to the skeleton via an oxygen atom, for example OCH₂CH₂, OCH₂CH₂CH₂ and OCH₂CH₂CH₂CH₂;
   oxyalkyleneoxy: divalent unbranched chains of 1 to 3 CH₂ groups, where both valencies are attached to the skeleton via an oxygen atom, for example OCH₂O, OCH₂CH₂O and OCH₂CH₂CH₂O.

The scope of the present invention includes the (R)- and (S)-isomers and the racemates of compounds of the formula I having chiral centers.

With a view to the intended use of the triazolopyrimidines of the formula I, particular preference is given to the following meanings of the substituents, in each case on their own or in combination:

Preference is given to compounds I in which R¹ is hydrogen or C₁-C₄-alkyl, such as hydrogen or methyl or ethyl, in particular hydrogen.

In addition, preference is given to compounds I in which R² is C₁-C₈-alkyl or C₃-C₆-cycloalkyl, in particular C₁-C₆-alkyl or C₃-C₆-cycloalkyl, preferably methyl, ethyl, isopropyl, isobutyl, tert-Butyl, sec-pentyl, cyclopropyl or cyclopentyl, in particular tert-butyl.

Preference is likewise given to compounds I in which R³ is hydrogen, C₁-C₈-alkyl or C₃-C₆-cycloalkyl, in particular hydrogen, C₁-C₆-alkyl or C₃-C₆-cycloalkyl, preferably hydrogen, methyl, ethyl, n-propyl or isopropyl. If R³ is an alkyl group, R³ preferably has the same meaning as R².

In a further embodiment of the compounds of the formula I, R² and R³ together form a C₃-C₆-alkylene group, in particular a C₃-C₄-alkylene group.

In a further embodiment of the compounds of the formula I, R⁴, R⁵, R⁶ and R⁷ are each hydrogen or C₁-C₄-alkyl, in particular hydrogen, methyl or ethyl, in particular hydrogen.

In a further embodiment of the compounds of the formula I, R⁴ and R⁵ and/or R⁶ and R⁷, in each case together, form a C₃-C₆-alkylene, C₃-C₆-oxyalkylene or C₂-C₅-oxyalkyleneoxy group, in particular a C₃-C₄-alkylene group.

In a preferred embodiment of the compounds of the formula I, the index p has the value zero.

In a further preferred embodiment of the compounds of the formula I, Y is oxygen.

In a further embodiment of the compounds of the formula I, Z is a monovalent group.

In a preferred embodiment of the compounds of the formula I, Z is C₁-C₄-alkyl or C₁-C₄-alkylcarbonyl, in particular methyl, ethyl, n-propyl, isopropyl, acetyl, propan-1-one or butan-1-one.

Preference is given to compounds I in which X is halogen, C₁-C₄-alkyl, cyano or C₁-C₄-alkoxy, such as chlorine, bromine, methyl, cyano, methoxy or ethoxy, in particular chlorine.

In a preferred embodiment of the compounds I, at least one group L is located in the ortho-position to the point of attachment to the triazolopyrimidine skeleton, in particular chlorine, fluorine or methyl.

Moreover, particular preference is given to compounds I in which the phenyl group substituted by Lₘ is the group A 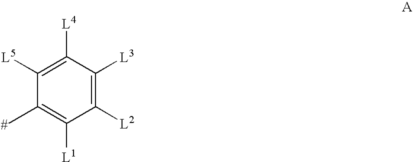
in which # is the point of attachment to the triazolopyrimidine skeleton and
L¹ is fluorine, chlorine, CH₃ or CF₃;
L², L⁴ independently of one another are hydrogen or fluorine;
L³ is hydrogen, fluorine, chlorine, CN, CH₃, OCH₃, NH₂, NHCH₃, N(CH₃)₂, C(O)NH₂ or C(O)CH₃; and
L⁵ is hydrogen, fluorine, chlorine or CH₃.

Particular preference is given to compounds I in which Lₘ is one of the following combinations of substituents: 2-fluoro-6-chloro, 2,6-difluoro, 2,6-dichloro, 2-fluoro-6-methyl, 2,4,6-trifluoro, 2,6-difluoro-4-methoxy, 2-chloro-4-methoxy, pentafluoro, 2-methyl-4-fluoro, 2-trifluoromethyl, 2-methoxy-6-fluoro, 2-chloro, 2-fluoro, 2,4-difluoro, 2-fluoro-4-chloro, 2-chloro-4-fluoro, 2-chloro-5-fluoro, 2,3-difluoro, 2,5-difluoro, 2,3,4-trifluoro, 2-methyl, 2,4-dimethyl, 2-methyl-4-chloro, 2-methyl-5-fluoro, 2-fluoro-4-methyl, 2,6-dimethyl, 2,4,6-trimethyl, 2,6-difluoro-4-methyl, 2-trifluoromethyl-4-fluoro, 2-trifluoromethyl-5-fluoro or 2-trifluoromethyl-5-chloro.

A further preferred embodiment of the invention relates to compounds of the formula I.1: 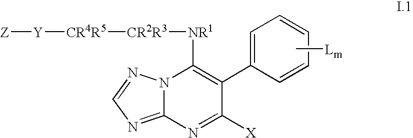
in which the variables are as defined above.

A further embodiment of the invention relates to compounds of the formula I.2, 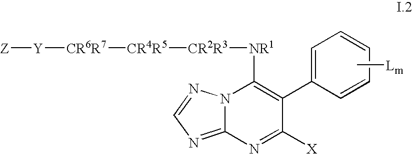
in which the variables are as defined above.

A further embodiment of the invention relates to compounds of the formula I and 1.1 in which Z is a group attached via a carbonyl group.

These compounds conform in particular to the formula I.3: 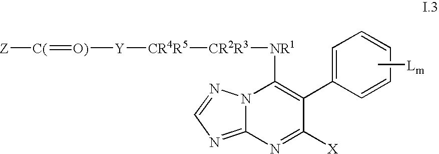
in which the variables are as defined according to the formula I and Z is defined as follows:
hydrogen, C₁-C₈-alkyl, C₁-C₈-alkoxy, C₃-C₆-cycloalkyl, C₃-C₈-alkenyloxy, C₃-C₈-alkynyloxy, C₃-C₆-cycloalkoxy, C₃-C₆-cycloalkenyloxy, NR^{A}R^{B}, a five- to ten-membered saturated, partially unsaturated or aromatic heterocycle which comprises one to four heteroatoms from the group consisting of O, N or S; the group Z may be partially or fully halogenated or carry one to three groups R^{b}.

In particular with a view to their use, preference is given to the compounds I compiled in the tables below. Moreover, the groups mentioned for a substituent in the tables are per se, independently of the combination in which they are mentioned, a particularly preferred embodiment of the substituent in question.

In addition to the compounds mentioned in tables 1 to 64, the corresponding derivatives in which X is cyano, methyl or methoxy also form a preferred subject matter.

Table 1

Compounds of the formula I.1 in which X is chlorine, Lₘ is 2-fluoro-6-chloro and R¹, R⁴ and R⁵ are hydrogen and the combination of R², R³, Y and Z corresponds for each compound to one row of table A

Table 2

Compounds of the formula I.1 in which X is chlorine, Lₘ is 2,6-difluoro and R¹, R⁴ and R⁵ are hydrogen and the combination of R², R³, Y and Z corresponds for each compound to one row of table A

Table 3

Compounds of the formula I.1 in which X is chlorine, Lₘ is 2,6-dichloro and R¹, R⁴ and R⁵ are hydrogen and the combination of R², R³, Y and Z corresponds for each compound to one row of table A

Table 4

Compounds of the formula I.1 in which X is chlorine, Lₘ is 2-fluoro-6-methyl and R¹, R⁴ and R⁵ are hydrogen and the combination of R², R³, Y and Z corresponds for each compound to one row of table A

Table 5

Compounds of the formula I.1 in which X is chlorine, Lₘ is 2,4,6-trifluoro and R¹, R⁴ and R⁵ are hydrogen and the combination of R², R³, Y and Z corresponds for each compound to one row of table A

Table 6

Compounds of the formula I.1 in which X is chlorine, Lₘ is 2,6-difluoro-4-methoxy and R¹, R⁴ and R⁵ are hydrogen and the combination of R², R³, Y and Z corresponds for each compound to one row of table A

Table 7

Compounds of the formula I.1 in which X is chlorine, Lₘ is pentafluoro and R¹, R⁴ and R⁵ are hydrogen and the combination of R², R³, Y and Z corresponds for each compound to one row of table A

Table 8

Compounds of the formula I.1 in which X is chlorine, Lₘ is 2-methyl-4-fluoro and R¹, R⁴ and R⁵ are hydrogen and the combination of R², R³, Y and Z corresponds for each compound to one row of table A

Table 9

Compounds of the formula I.1 in which X is chlorine, Lₘ is 2-trifluoromethyl and R¹, R⁴ and R⁵ are hydrogen and the combination of R², R³, Y and Z corresponds for each compound to one row of table A

Table 10

Compounds of the formula I.1 in which X is chlorine, Lₘ is 2-methoxy-6-fluoro and R¹, R⁴ and R⁵ are hydrogen and the combination of R², R³, Y and Z corresponds for each compound to one row of table A

Table 11

Compounds of the formula I.1 in which X is chlorine, Lₘ is 2-chloro and R¹, R⁴ and R⁵ are hydrogen and the combination of R², R³, Y and Z corresponds for each compound to one row of table A

Table 12

Compounds of the formula I.1 in which X is chlorine, Lₘ is 2-fluoro and R¹, R⁴ and R⁵ are hydrogen and the combination of R², R³, Y and Z corresponds for each compound to one row of table A

Table 13

Compounds of the formula I.1 in which X is chlorine, Lₘ is 2,4-difluoro and R¹, R⁴ and R⁵ are hydrogen and the combination of R², R³, Y and Z corresponds for each compound to one row of table A

Table 14

Compounds of the formula I.1 in which X is chlorine, Lₘ is 2-fluoro-4-chloro and R¹, R⁴ and R⁵ are hydrogen and the combination of R², R³, Y and Z corresponds for each compound to one row of table A

Table 15

Compounds of the formula I.1 in which X is chlorine, Lₘ is 2-chloro-4-fluoro and R¹, R⁴ and R⁵ are hydrogen and the combination of R², R³, Y and Z corresponds for each compound to one row of table A

Table 16

Compounds of the formula I.1 in which X is chlorine, Lₘ is 2,3-difluoro and R¹, R⁴ and R⁵ are hydrogen and the combination of R², R³, Y and Z corresponds for each compound to one row of table A

Table 17

Compounds of the formula I.1 in which X is chlorine, Lₘ is 2,5-difluoro and R¹, R⁴ and R⁵ are hydrogen and the combination of R², R³, Y and Z corresponds for each compound to one row of table A

Table 18

Compounds of the formula I.1 in which X is chlorine, Lₘ is 2,3,4-trifluoro and R¹, R⁴ and R⁵ are hydrogen and the combination of R², R³, Y and Z corresponds for each compound to one row of table A

Table 19

Compounds of the formula I.1 in which X is chlorine, Lₘ is 2-methyl and R¹, R⁴ and R⁵ are hydrogen and the combination of R², R³, Y and Z corresponds for each compound to one row of table A

Table 20

Compounds of the formula I.1 in which X is chlorine, Lₘ is 2,4-dimethyl and R¹, R⁴ and R⁵ are hydrogen and the combination of R², R³, Y and Z corresponds for each compound to one row of table A

Table 21

Compounds of the formula I.1 in which X is chlorine, Lₘ is 2-methyl-4-chloro and R¹, R⁴ and R⁵ are hydrogen and the combination of R², R³, Y and Z corresponds for each compound to one row of table A

Table 22

Compounds of the formula I.1 in which X is chlorine, Lₘ is 2-fluoro-4-methyl and R¹, R⁴ and R⁵ are hydrogen and the combination of R², R³, Y and Z corresponds for each compound to one row of table A

Table 23

Compounds of the formula I.1 in which X is chlorine, Lₘ is 2,6-dimethyl and R¹, R⁴ and R⁵ are hydrogen and the combination of R², R³, Y and Z corresponds for each compound to one row of table A

Table 24

Compounds of the formula I.1 in which X is chlorine, Lₘ is 2,4,6-trimethyl and R¹, R⁴ and R⁵ are hydrogen and the combination of R², R³, Y and Z corresponds for each compound to one row of table A

Table 25

Compounds of the formula I.1 in which X is chlorine, Lₘ is 2,6-difluoro-4-methyl and R¹, R⁴ and R⁵ are hydrogen and the combination of R², R³, Y and Z corresponds for each compound to one row of table A

Table 26

Compounds of the formula I.1 in which X is chlorine, Lₘ is 2-trifluoromethyl-4-fluoro and R¹, R⁴ and R⁵ are hydrogen and the combination of R², R³, Y and Z corresponds for each compound to one row of table A

Table 27

Compounds of the formula I.1 in which X is chlorine, Lₘ is 2-trifluoromethyl-5-fluoro and R¹, R⁴ and R⁵ are hydrogen and the combination of R², R³, Y and Z corresponds for each compound to one row of table A

Table 28

Compounds of the formula I.1 in which X is chlorine, Lₘ is 2-trifluoromethyl-5-chloro and R¹, R⁴ and R⁵ are hydrogen and the combination of R², R³, Y and Z corresponds for each compound to one row of table A

Table 29

Compounds of the formula I.1 in which X is chlorine, Lₘ is 2-chloro-4-fluoro and R¹, R⁴ and R⁵ are hydrogen and the combination of R², R³, Y and Z corresponds for each compound to one row of table A

Table 30

Compounds of the formula I.1 in which X is chlorine, Lₘ is 2-chloro-5-fluoro and R¹, R⁴ and R⁵ are hydrogen and the combination of R², R³, Y and Z corresponds for each compound to one row of table A

Table 31

Compounds of the formula I.1 in which X is chlorine, Lₘ is 2-methyl-5-fluoro and R¹, R⁴ and R⁵ are hydrogen and the combination of R², R³, Y and Z corresponds for each compound to one row of table A

Table 32

Compounds of the formula I.1 in which X is chlorine, Lₘ is 2-chloro-4-methoxy and R¹, R⁴ and R⁵ are hydrogen and the combination of R², R³, Y and Z corresponds for each compound to one row of table A

Table 33

Compounds of the formula I.1 in which X is chlorine, Lₘ is 2-fluoro-6-chloro and R¹ is methyl, R⁴ and R⁵ are hydrogen and the combination of R², R³, Y and Z corresponds for each compound to one row of table A

Table 34

Compounds of the formula I.1 in which X is chlorine, Lₘ is 2,6-difluoro and R¹ is methyl, R⁴ and R⁵ are hydrogen and the combination of R², R³, Y and Z corresponds for each compound to one row of table A

Table 35

Compounds of the formula I.1 in which X is chlorine, Lₘ is 2,6-dichloro and R¹ is methyl, R⁴ and R⁵ are hydrogen and the combination of R², R³, Y and Z corresponds for each compound to one row of table A

Table 36

Compounds of the formula I.1 in which X is chlorine, Lₘ is 2-fluoro-6-methyl and R¹ is methyl, R⁴ and R⁵ are hydrogen and the combination of R², R³, Y and Z corresponds for each compound to one row of table A

Table 37

Compounds of the formula I.1, in which X is chlorine, Lₘ is 2,4,6-trifluoro and R¹ is methyl, R⁴ and R⁵ are hydrogen and the combination of R², R³, Y and Z corresponds for each compound to one row of table A

Table 38

Compounds of the formula I.1 in which X is chlorine, Lₘ is 2,6-difluoro-4-methoxy and R¹ is methyl, R⁴ and R⁵ are hydrogen and the combination of R², R³, Y and Z corresponds for each compound to one row of table A

Table 39

Compounds of the formula I.1 in which X is chlorine, Lₘ is pentafluoro and R¹ is methyl, R⁴ and R⁵ are hydrogen and the combination of R², R³, Y and Z corresponds for each compound to one row of table A

Table 40

Compounds of the formula I.1 in which X is chlorine, Lₘ is 2-methyl-4-fluoro and R¹ is methyl, R⁴ and R⁵ are hydrogen and the combination of R², R³, Y and Z corresponds for each compound to one row of table A

Table 41

Compounds of the formula I.1 in which X is chlorine, Lₘ is 2-trifluoromethyl and R¹ is methyl, R⁴ and R⁵ are hydrogen and the combination of R², R³, Y and Z corresponds for each compound to one row of table A

Table 42

Compounds of the formula I.1 in which X is chlorine, Lₘ is 2-methoxy-6-fluoro and R¹ is methyl, R⁴ and R⁵ are hydrogen and the combination of R², R³, Y and Z corresponds for each compound to one row of table A

Table 43

Compounds of the formula I.1 in which X is chlorine, Lₘ is 2-chloro and R¹ is methyl, R⁴ and R⁵ are hydrogen and the combination of R², R³, Y and Z corresponds for each compound to one row of table A

Table 44

Compounds of the formula I.1 in which X is chlorine, Lₘ is 2-fluoro and R¹ is methyl, R⁴ and R⁵ are hydrogen and the combination of R², R³, Y and Z corresponds for each compound to one row of table A

Table 45

Compounds of the formula I.1 in which X is chlorine, Lₘ is 2,4-difluoro and R¹ is methyl, R⁴ and R⁵ are hydrogen and the combination of R², R³, Y and Z corresponds for each compound to one row of table A

Table 46

Compounds of the formula I.1, in which X is chlorine, Lₘ is 2-fluoro-4-chloro and R¹ is methyl, R⁴ and R⁵ are hydrogen and the combination of R², R³, Y and Z corresponds for each compound to one row of table A

Table 47

Compounds of the formula I.1 in which X is chlorine, Lₘ is 2-chloro-4-fluoro and R¹ is methyl, R⁴ and R⁵ are hydrogen and the combination of R², R³, Y and Z corresponds for each compound to one row of table A

Table 48

Compounds of the formula I.1 in which X is chlorine, Lₘ is 2,3-difluoro and R¹ is methyl, R⁴ and R⁵ are hydrogen and the combination of R², R³, Y and Z corresponds for each compound to one row of table A

Table 49

Compounds of the formula I.1 in which X is chlorine, Lₘ is 2,5-difluoro and R¹ is methyl, R⁴ and R⁵ are hydrogen and the combination of R², R³, Y and Z corresponds for each compound to one row of table A

Table 50

Compounds of the formula I.1 in which X is chlorine, Lₘ is 2,3,4-trifluoro and R¹ is methyl, R⁴ and R⁵ are hydrogen and the combination of R², R³, Y and Z corresponds for each compound to one row of table A

Table 51

Compounds of the formula I.1 in which X is chlorine, Lₘ is 2-methyl and R¹ is methyl, R⁴ and R⁵ are hydrogen and the combination of R², R³, Y and Z corresponds for each compound to one row of table A

Table 52

Compounds of the formula I.1 in which X is chlorine, Lₘ is 2,4-dimethyl and R¹ is methyl, R⁴ and R⁵ are hydrogen and the combination of R², R³, Y and Z corresponds for each compound to one row of table A

Table 53

Compounds of the formula I.1 in which X is chlorine, Lₘ is 2-methyl-4-chloro and R¹ is methyl, R⁴ and R⁵ are hydrogen and the combination of R², R³, Y and Z corresponds for each compound to one row of table A

Table 54

Compounds of the formula I.1 in which X is chlorine, Lₘ is 2-fluoro-4-methyl and R¹ is methyl, R⁴ and R⁵ are hydrogen and the combination of R², R³, Y and Z corresponds for each compound to one row of table A

Table 55

Compounds of the formula I.1 in which X is chlorine, Lₘ is 2,6-dimethyl and R¹ is methyl, R⁴ and R⁵ are hydrogen and the combination of R², R³, Y and Z corresponds for each compound to one row of table A

Table 56

Compounds of the formula I.1 in which X is chlorine, Lₘ is 2,4,6-trimethyl and R¹ is methyl, R⁴ and R⁵ are hydrogen and the combination of R², R³, Y and Z corresponds for each compound to one row of table A

Table 57

Compounds of the formula I.1 in which X is chlorine, Lₘ is 2,6-difluoro-4-methyl and R¹ is methyl, R⁴ and R⁵ are hydrogen and the combination of R², R³, Y and Z corresponds for each compound to one row of table A

Table 58

Compounds of the formula I.1 in which X is chlorine, Lₘ is 2-trifluoromethyl-4-fluoro and R¹ is methyl, R⁴ and R⁵ are hydrogen and the combination of R², R³, Y and Z corresponds for each compound to one row of table A

Table 59

Compounds of the formula I.1 in which X is chlorine, Lₘ is 2-trifluoromethyl-5-fluoro and R¹ is methyl, R⁴ and R⁵ are hydrogen and the combination of R², R³, Y and Z corresponds for each compound to one row of table A

Table 60

Compounds of the formula I.1 in which X is chlorine, Lₘ is 2-trifluoromethyl-5-chloro and R¹ is methyl, R⁴ and R⁵ are hydrogen and the combination of R², R³, Y and Z corresponds for each compound to one row of table A

Table 61

Compounds of the formula I.1 in which X is chlorine, Lₘ is 2-chloro-4-fluoro and R¹ is methyl, R⁴ and R⁵ are hydrogen and the combination of R², R³, Y and Z corresponds for each compound to one row of table A

Table 62

Compounds of the formula I.1 in which X is chlorine, Lₘ is 2-chloro-5-fluoro and R¹ is methyl, R⁴ and R⁵ are hydrogen and the combination of R², R³, Y and Z corresponds for each compound to one row of table A

Table 63

Compounds of the formula I.1 in which X is chlorine, Lₘ is 2-methyl-5-fluoro and R¹ is methyl, R⁴ and R⁵ are hydrogen and the combination of R², R³, Y and Z corresponds for each compound to one row of table A

Table 64

Compounds of the formula I.1 in which X is chlorine, Lₘ is 2-chloro-4-methoxy and R¹ is methyl, R⁴ and R⁵ are hydrogen and the combination of R², R³, Y and Z corresponds for each compound to one row of table A

Table 65

Compounds of the formula I.3, in which X is chlorine, Lₘ is 2-fluoro-6-chloro and R¹, R⁴ and R⁵ are hydrogen and the combination of R², R³, Y and Z corresponds for each compound to one row of table B

Table 66

Compounds of the formula I.3, in which X is chlorine, Lₘ is 2,6-difluoro and R¹, R⁴ and R⁵ are hydrogen and the combination of R², R³, Y and Z corresponds for each compound to one row of table B

Table 67

Compounds of the formula I.3, in which X is chlorine, Lₘ is 2,6-dichloro and R¹, R⁴ and R⁵ are hydrogen and the combination of R², R³, Y and Z corresponds for each compound to one row of table B

Table 68

Compounds of the formula I.3, in which X is chlorine, Lₘ is 2-fluoro-6-methyl and R¹, R⁴ and R⁵ are hydrogen and the combination of R², R³, Y and Z corresponds for each compound to one row of table B

Table 69

Compounds of the formula I.3, in which X is chlorine, Lₘ is 2,4,6-trifluoro and R¹, R⁴ and R⁵ are hydrogen and the combination of R², R³, Y and Z corresponds for each compound to one row of table B

Table 70

Compounds of the formula I.3, in which X is chlorine, Lₘ is 2,6-difluoro-4-methoxy and R¹, R⁴ and R⁵ are hydrogen and the combination of R², R³, Y and Z corresponds for each compound to one row of table B

Table 71

Compounds of the formula I.3, in which X is chlorine, Lₘ is pentafluoro and R¹, R⁴ and R⁵ are hydrogen and the combination of R², R³, Y and Z corresponds for each compound to one row of table B

Table 72

Compounds of the formula I.3, in which X is chlorine, Lₘ is 2-methyl-4-fluoro and R¹, R⁴ and R⁵ are hydrogen and the combination of R², R³, Y and Z corresponds for each compound to one row of table B

Table 73

Compounds of the formula I.3, in which X is chlorine, Lₘ is 2-trifluoromethyl and R¹, R⁴ and R⁵ are hydrogen and the combination of R², R³, Y and Z corresponds for each compound to one row of table B

Table 74

Compounds of the formula I.3, in which X is chlorine, Lₘ is 2-methoxy-6-fluoro and R¹, R⁴ and R⁵ are hydrogen and the combination of R², R³, Y and Z corresponds for each compound to one row of table B

Table 75

Compounds of the formula I.3, in which X is chlorine, Lₘ is 2-chloro and R¹, R⁴ and R⁵ are hydrogen and the combination of R², R³, Y and Z corresponds for each compound to one row of table B

Table 76

Compounds of the formula I.3, in which X is chlorine, Lₘ is 2-fluoro and R¹, R⁴ and R⁵ are hydrogen and the combination of R², R³, Y and Z corresponds for each compound to one row of table B

Table 77

Compounds of the formula I.3, in which X is chlorine, Lₘ is 2,4-difluoro and R¹, R⁴ and R⁵ are hydrogen and the combination of R², R³, Y and Z corresponds for each compound to one row of table B

Table 78

Compounds of the formula I.3, in which X is chlorine, Lₘ is 2-fluoro-4-chloro and R¹, R⁴ and R⁵ are hydrogen and the combination of R², R³, Y and Z corresponds for each compound to one row of table B

Table 79

Compounds of the formula I.3, in which X is chlorine, Lₘ is 2-chloro-4-fluoro and R¹, R⁴ and R⁵ are hydrogen and the combination of R², R³, Y and Z corresponds for each compound to one row of table B

Table 80

Compounds of the formula I.3, in which X is chlorine, Lₘ is 2,3-difluoro and R¹, R⁴ and R⁵ are hydrogen and the combination of R², R³, Y and Z corresponds for each compound to one row of table B

Table 81

Compounds of the formula I.3, in which X is chlorine, Lₘ is 2,5-difluoro and R¹, R⁴ and R⁵ are hydrogen and the combination of R², R³, Y and Z corresponds for each compound to one row of table B

Table 82

Compounds of the formula I.3, in which X is chlorine, Lₘ is 2,3,4-trifluoro and R¹, R⁴ and R⁵ are hydrogen and the combination of R², R³, Y and 7 corresponds for each compound to one row of table B

Table 83

Compounds of the formula I.3, in which X is chlorine, Lₘ is 2-methyl and R¹, R⁴ and R⁵ are hydrogen and the combination of R², R³, Y and Z corresponds for each compound to one row of table B

Table 84

Compounds of the formula I.3, in which X is chlorine, Lₘ is 2,4-dimethyl and R¹, R⁴ and R⁵ are hydrogen and the combination of R², R³, Y and Z corresponds for each compound to one row of table B

Table 85

Compounds of the formula I.3, in which X is chlorine, Lₘ is 2-methyl-4-chloro and R¹, R⁴ and R⁵ are hydrogen and the combination of R², R³, Y and Z corresponds for each compound to one row of table B

Table 86

Compounds of the formula I.3, in which X is chlorine, Lₘ is 2-fluoro-4-methyl and R¹, R⁴ and R⁵ are hydrogen and the combination of R², R³, Y and Z corresponds for each compound to one row of table B

Table 87

Compounds of the formula I.3, in which X is chlorine, Lₘ is 2,6-dimethyl and R¹, R⁴ and R⁵ are hydrogen and the combination of R², R³, Y and Z corresponds for each compound to one row of table B

Table 88

Compounds of the formula I.3, in which X is chlorine, Lₘ is 2,4,6-trimethyl and R¹, R⁴ and R⁵ are hydrogen and the combination of R², R³, Y and Z corresponds for each compound to one row of table B

Table 89

Compounds of the formula I.3, in which X is chlorine, Lₘ is 2,6-difluoro-4-methyl and R¹, R⁴ and R⁵ are hydrogen and the combination of R², R³, Y and Z corresponds for each compound to one row of table B

Table 90

Compounds of the formula I.3, in which X is chlorine, Lₘ is 2-trifluoromethyl-4-fluoro and R¹, R⁴ and R⁵ are hydrogen and the combination of R², R³, Y and Z corresponds for each compound to one row of table B

Table 91

Compounds of the formula I.3, in which X is chlorine, Lₘ is 2-trifluoromethyl-5-fluoro and R¹, R⁴ and R⁵ are hydrogen and the combination of R², R³, Y and Z corresponds for each compound to one row of table B

Table 92

Compounds of the formula I.3, in which X is chlorine, Lₘ is 2-trifluoromethyl-5-chloro and R¹, R⁴ and R⁵ are hydrogen and the combination of R², R³, Y and Z corresponds for each compound to one row of table B

Table 93

Compounds of the formula I.3, in which X is chlorine, Lₘ is 2-chloro-4-fluoro and R¹, R⁴ and R⁵ are hydrogen and the combination of R², R³, Y and Z corresponds for each compound to one row of table B

Table 94

Compounds of the formula I.3, in which X is chlorine, Lₘ is 2-chloro-5-fluoro and R¹, R⁴ and R⁵ are hydrogen and the combination of R², R³, Y and Z corresponds for each compound to one row of table B

Table 95

Compounds of the formula I.3, in which X is chlorine, Lₘ is 2-methyl-5-fluoro and R¹, R⁴ and R⁵ are hydrogen and the combination of R², R³, Y and Z corresponds for each compound to one row of table B

Table 96

Compounds of the formula I.3, in which X is chlorine, Lₘ is 2-chloro-4-methoxy and R¹, R⁴ and R⁵ are hydrogen and the combination of R², R³, Y and Z corresponds for each compound to one row of table B

TABLE A

| No. | R² | R³ | Y | Z |
|---|---|---|---|---|
| A-1 | CH₃ | H | O | H |
| A-2 | CH₃ | H | O | CH₃ |
| A-3 | CH₃ | H | O | CH₂CH₃ |
| A-4 | CH₃ | H | O | CH₂CH₂CH₃ |
| A-5 | CH₃ | H | O | CH₂CH₂CH₂CH₃ |
| A-6 | CH₃ | H | O | CH(CH₃)CH₂CH₃ |
| A-7 | CH₃ | H | O | CH₂CH₂(CH₃)₂ |
| A-8 | CH₃ | H | O | C(CH₃)₃ |
| A-9 | CH₃ | H | O | CH(CH₃)₂ |
| A-10 | CH₃ | H | O | C(O)CH₃ |
| A-11 | CH₃ | H | O | C(O)CH₂CH₃ |
| A-12 | CH₃ | H | O | C(O)CH₂CH₂CH₃ |
| A-13 | CH₃ | H | O | C(O)CH₂CH₂CH₂CH₃ |
| A-14 | CH₃ | H | O | C(O)CH(CH₂CH₃)₂ |
| A-15 | CH₃ | H | O | C(O)CH(CH₂F)₂ |
| A-16 | CH₃ | H | O | C(O)CH₂CH₂C₆H₅ |
| A-17 | CH₃ | H | O | C(O)C₆H₅ |
| A-18 | CH₃ | H | O | C(O)(4-OCH₃—C₆H₄) |
| A-19 | CH₃ | H | O | C(O)(4-Cl—C₆H₄) |
| A-20 | CH₃ | H | O | C(O)(4-Cl—C₆H₄) |
| A-21 | CH₃ | H | O | C(O)(4-pyridyl) |
| A-22 | CH₃ | H | O | C(O)(2-CH₂CH₃—C₆H₄) |
| A-23 | CH₃ | H | O | C(O)(2-CH₃—C₆H₄) |
| A-24 | CH₃ | H | O | C(O)(3-CH₃—C₆H₄) |
| A-25 | CH₃ | H | O | C(O)(4-CH₃—C₆H₄) |
| A-26 | CH₃ | CH₃ | O | H |
| A-27 | CH₃ | CH₃ | O | CH₃ |
| A-28 | CH₃ | CH₃ | O | CH₂CH₃ |
| A-29 | CH₃ | CH₃ | O | CH₂CH₂CH₃ |
| A-30 | CH₃ | CH₃ | O | CH(CH₃)₂ |
| A-31 | CH₃ | CH₃ | O | CH₂CH₂CH₂CH₃ |
| A-32 | CH₃ | CH₃ | O | CH(CH₃)CH₂CH₃ |
| A-33 | CH₃ | CH₃ | O | CH₂CH₂(CH₃)₂ |
| A-34 | CH₃ | CH₃ | O | C(CH₃)₃ |
| A-35 | CH₃ | CH₃ | O | C(O)CH₃ |
| A-36 | CH₃ | CH₃ | O | C(O)CH₂CH₃ |
| A-37 | CH₃ | CH₃ | O | C(O)CH₂CH₂CH₃ |
| A-38 | CH₃ | CH₃ | O | C(O)CH₂CH₂CH₂CH₃ |
| A-39 | CH₃ | CH₃ | O | C(O)CH(CH₂CH₃)₂ |
| A-40 | CH₃ | CH₃ | O | C(O)CH(CH₂F)₂ |
| A-41 | CH₃ | CH₃ | O | C(O)CH₂CH₂C₆H₅ |
| A-42 | CH₃ | CH₃ | O | C(O)C₆H₅ |
| A-43 | CH₃ | CH₃ | O | C(O)(4-OCH₃—C₆H₄) |
| A-44 | CH₃ | CH₃ | O | C(O)(4-Cl—C₆H₄) |
| A-45 | CH₃ | CH₃ | O | C(O)(4-Cl—C₆H₄) |
| A-46 | CH₃ | CH₃ | O | C(O)(4-pyridyl) |
| A-47 | CH₃ | CH₃ | O | C(O)(2-CH₂CH₃—C₆H₄) |
| A-48 | CH₃ | CH₃ | O | C(O)(2-CH₃—C₆H₄) |
| A-49 | CH₃ | CH₃ | O | C(O)(3-CH₃—C₆H₄) |
| A-50 | CH₃ | CH₃ | O | C(O)(4-CH₃—C₆H₄) |
| A-51 | CH₂CH₃ | H | O | H |
| A-52 | CH₂CH₃ | H | O | CH₃ |
| A-53 | CH₂CH₃ | H | O | CH₂CH₃ |
| A-54 | CH₂CH₃ | H | O | CH₂CH₂CH₃ |
| A-55 | CH₂CH₃ | H | O | CH(CH₃)₂ |
| A-56 | CH₂CH₃ | H | O | CH₂CH₂CH₂CH₃ |
| A-57 | CH₂CH₃ | H | O | CH(CH₃)CH₂CH₃ |
| A-58 | CH₂CH₃ | H | O | CH₂CH₂(CH₃)₂ |
| A-59 | CH₂CH₃ | H | O | C(CH₃)₃ |
| A-60 | CH₂CH₃ | H | O | C(O)CH₃ |
| A-61 | CH₂CH₃ | H | O | C(O)CH₂CH₃ |
| A-62 | CH₂CH₃ | H | O | C(O)CH₂CH₂CH₃ |
| A-63 | CH₂CH₃ | H | O | C(O)CH₂CH₂CH₂CH₃ |
| A-64 | CH₂CH₃ | H | O | C(O)CH(CH₂CH₃)₂ |
| A-65 | CH₂CH₃ | H | O | C(O)CH(CH₂F)₂ |
| A-66 | CH₂CH₃ | H | O | C(O)CH₂CH₂C₆H₅ |
| A-67 | CH₂CH₃ | H | O | C(O)C₆H₅ |
| A-68 | CH₂CH₃ | H | O | C(O)(4-OCH₃—C₆H₄) |
| A-69 | CH₂CH₃ | H | O | C(O)(4-Cl—C₆H₄) |
| A-70 | CH₂CH₃ | H | O | C(O)(4-Cl—C₆H₄) |
| A-71 | CH₂CH₃ | H | O | C(O)(4-pyridyl) |
| A-72 | CH₂CH₃ | H | O | C(O)(2-CH₂CH₃—C₆H₄) |
| A-73 | CH₂CH₃ | H | O | C(O)(2-CH₃—C₆H₄) |
| A-74 | CH₂CH₃ | H | O | C(O)(3-CH₃—C₆H₄) |
| A-75 | CH₂CH₃ | H | O | C(O)(4-CH₃—C₆H₄) |
| A-76 | CH₂CH₃ | CH₂CH₃ | O | H |
| A-77 | CH₂CH₃ | CH₂CH₃ | O | CH₃ |
| A-78 | CH₂CH₃ | CH₂CH₃ | O | CH₂CH₃ |
| A-79 | CH₂CH₃ | CH₂CH₃ | O | CH₂CH₂CH₃ |
| A-80 | CH₂CH₃ | CH₂CH₃ | O | CH(CH₃)₂ |
| A-81 | CH₂CH₃ | CH₂CH₃ | O | CH₂CH₂CH₂CH₃ |
| A-82 | CH₂CH₃ | CH₂CH₃ | O | CH(CH₃)CH₂CH₃ |
| A-83 | CH₂CH₃ | CH₂CH₃ | O | CH₂CH₂(CH₃)₂ |
| A-84 | CH₂CH₃ | CH₂CH₃ | O | C(CH₃)₃ |
| A-85 | CH₂CH₃ | CH₂CH₃ | O | C(O)CH₃ |
| A-86 | CH₂CH₃ | CH₂CH₃ | O | C(O)CH₂CH₃ |
| A-87 | CH₂CH₃ | CH₂CH₃ | O | C(O)CH₂CH₂CH₃ |
| A-88 | CH₂CH₃ | CH₂CH₃ | O | C(O)CH₂CH₂CH₂CH₃ |
| A-89 | CH₂CH₃ | CH₂CH₃ | O | C(O)CH(CH₂CH₃)₂ |
| A-90 | CH₂CH₃ | CH₂CH₃ | O | C(O)CH(CH₂F)₂ |
| A-91 | CH₂CH₃ | CH₂CH₃ | O | C(O)CH₂CH₂C₆H₅ |
| A-92 | CH₂CH₃ | CH₂CH₃ | O | C(O)C₆H₅ |
| A-93 | CH₂CH₃ | CH₂CH₃ | O | C(O)(4-OCH₃—C₆H₄) |
| A-94 | CH₂CH₃ | CH₂CH₃ | O | C(O)(4-Cl—C₆H₄) |
| A-95 | CH₂CH₃ | CH₂CH₃ | O | C(O)(4-Cl—C₆H₄) |
| A-96 | CH₂CH₃ | CH₂CH₃ | O | C(O)(4-pyridyl) |
| A-97 | CH₂CH₃ | CH₂CH₃ | O | C(O)(2-CH₂CH₃—C₆H₄) |
| A-98 | CH₂CH₃ | CH₂CH₃ | O | C(O)(2-CH₃—C₆H₄) |
| A-99 | CH₂CH₃ | CH₂CH₃ | O | C(O)(3-CH₃—C₆H₄) |
| A-100 | CH₂CH₃ | CH₂CH₃ | O | C(O)(4-CH₃—C₆H₄) |
| A-101 | CH₂CH₂CH₃ | H | O | H |
| A-102 | CH₂CH₂CH₃ | H | O | CH₃ |
| A-103 | CH₂CH₂CH₃ | H | O | CH₂CH₃ |
| A-104 | CH₂CH₂CH₃ | H | O | CH₂CH₂CH₃ |
| A-105 | CH₂CH₂CH₃ | H | O | CH(CH₃)₂ |
| A-106 | CH₂CH₂CH₃ | H | O | CH₂CH₂CH₂CH₃ |
| A-107 | CH₂CH₂CH₃ | H | O | CH(CH₃)CH₂CH₃ |
| A-108 | CH₂CH₂CH₃ | H | O | CH₂CH₂(CH₃)₂ |
| A-109 | CH₂CH₂CH₃ | H | O | C(CH₃)₃ |
| A-110 | CH₂CH₂CH₃ | H | O | C(O)CH₃ |
| A-111 | CH₂CH₂CH₃ | H | O | C(O)CH₂CH₃ |
| A-112 | CH₂CH₂CH₃ | H | O | C(O)CH₂CH₂CH₃ |
| A-113 | CH₂CH₂CH₃ | H | O | C(O)CH₂CH₂CH₂CH₃ |
| A-114 | CH₂CH₂CH₃ | H | O | C(O)CH(CH₂CH₃)₂ |
| A-115 | CH₂CH₂CH₃ | H | O | C(O)CH(CH₂F)₂ |
| A-116 | CH₂CH₂CH₃ | H | O | C(O)CH₂CH₂C₆H₅ |
| A-117 | CH₂CH₂CH₃ | H | O | C(O)C₆H₅ |
| A-118 | CH₂CH₂CH₃ | H | O | C(O)(4-OCH₃—C₆H₄) |
| A-119 | CH₂CH₂CH₃ | H | O | C(O)(4-Cl—C₆H₄) |
| A-120 | CH₂CH₂CH₃ | H | O | C(O)(4-Cl—C₆H₄) |
| A-121 | CH₂CH₂CH₃ | H | O | C(O)(4-pyridyl) |
| A-122 | CH₂CH₂CH₃ | H | O | C(O)(2-CH₂CH₃—C₆H₄) |
| A-123 | CH₂CH₂CH₃ | H | O | C(O)(2-CH₃—C₆H₄) |
| A-124 | CH₂CH₂CH₃ | H | O | C(O)(3-CH₃—C₆H₄) |
| A-125 | CH₂CH₂CH₃ | H | O | C(O)(4-CH₃—C₆H₄) |
| A-126 | CH₂CH₂CH₃ | CH₂CH₂CH₃ | O | H |
| A-127 | CH₂CH₂CH₃ | CH₂CH₂CH₃ | O | CH₃ |
| A-128 | CH₂CH₂CH₃ | CH₂CH₂CH₃ | O | CH₂CH₃ |
| A-129 | CH₂CH₂CH₃ | CH₂CH₂CH₃ | O | CH₂CH₂CH₃ |
| A-130 | CH₂CH₂CH₃ | CH₂CH₂CH₃ | O | CH(CH₃)₂ |
| A-131 | CH₂CH₂CH₃ | CH₂CH₂CH₃ | O | CH₂CH₂CH₂CH₃ |
| A-132 | CH₂CH₂CH₃ | CH₂CH₂CH₃ | O | CH(CH₃)CH₂CH₃ |
| A-133 | CH₂CH₂CH₃ | CH₂CH₂CH₃ | O | CH₂CH₂(CH₃)₂ |
| A-134 | CH₂CH₂CH₃ | CH₂CH₂CH₃ | O | C(CH₃)₃ |
| A-135 | CH₂CH₂CH₃ | CH₂CH₂CH₃ | O | C(O)CH₃ |
| A-136 | CH₂CH₂CH₃ | CH₂CH₂CH₃ | O | C(O)CH₂CH₃ |
| A-137 | CH₂CH₂CH₃ | CH₂CH₂CH₃ | O | C(O)CH₂CH₂CH₃ |
| A-138 | CH₂CH₂CH₃ | CH₂CH₂CH₃ | O | C(O)CH₂CH₂CH₂CH₃ |
| A-139 | CH₂CH₂CH₃ | CH₂CH₂CH₃ | O | C(O)CH(CH₂CH₃)₂ |
| A-140 | CH₂CH₂CH₃ | CH₂CH₂CH₃ | O | C(O)CH(CH₂F)₂ |
| A-141 | CH₂CH₂CH₃ | CH₂CH₂CH₃ | O | C(O)CH₂CH₂C₆H₅ |
| A-142 | CH₂CH₂CH₃ | CH₂CH₂CH₃ | O | C(O)C₆H₅ |
| A-143 | CH₂CH₂CH₃ | CH₂CH₂CH₃ | O | C(O)(4-OCH₃—C₆H₄) |
| A-144 | CH₂CH₂CH₃ | CH₂CH₂CH₃ | O | C(O)(4-Cl—C₆H₄) |
| A-145 | CH₂CH₂CH₃ | CH₂CH₂CH₃ | O | C(O)(4-Cl—C₆H₄) |
| A-146 | CH₂CH₂CH₃ | CH₂CH₂CH₃ | O | C(O)(4-pyridyl) |
| A-147 | CH₂CH₂CH₃ | CH₂CH₂CH₃ | O | C(O)(2-CH₂CH₃—C₆H₄) |
| A-148 | CH₂CH₂CH₃ | CH₂CH₂CH₃ | O | C(O)(2-CH₃—C₆H₄) |
| A-149 | CH₂CH₂CH₃ | CH₂CH₂CH₃ | O | C(O)(3-CH₃—C₆H₄) |
| A-150 | CH₂CH₂CH₃ | CH₂CH₂CH₃ | O | C(O)(4-CH₃—C₆H₄) |
| A-151 | CH(CH₃)₂ | H | O | H |
| A-152 | CH(CH₃)₂ | H | O | CH₃ |
| A-153 | CH(CH₃)₂ | H | O | CH₂CH₃ |
| A-154 | CH(CH₃)₂ | H | O | CH₂CH₂CH₃ |
| A-155 | CH(CH₃)₂ | H | O | CH(CH₃)₂ |
| A-156 | CH(CH₃)₂ | H | O | CH₂CH₂CH₂CH₃ |
| A-157 | CH(CH₃)₂ | H | O | CH(CH₃)CH₂CH₃ |
| A-158 | CH(CH₃)₂ | H | O | CH₂CH₂(CH₃)₂ |
| A-159 | CH(CH₃)₂ | H | O | C(CH₃)₃ |
| A-160 | CH(CH₃)₂ | H | O | C(O)CH₃ |
| A-161 | CH(CH₃)₂ | H | O | C(O)CH₂CH₃ |
| A-162 | CH(CH₃)₂ | H | O | C(O)CH₂CH₂CH₃ |
| A-163 | CH(CH₃)₂ | H | O | C(O)CH₂CH₂CH₂CH₃ |
| A-164 | CH(CH₃)₂ | H | O | C(O)CH(CH₂CH₃)₂ |
| A-165 | CH(CH₃)₂ | H | O | C(O)CH(CH₂F)₂ |
| A-166 | CH(CH₃)₂ | H | O | C(O)CH₂CH₂C₆H₅ |
| A-167 | CH(CH₃)₂ | H | O | C(O)C₆H₅ |
| A-168 | CH(CH₃)₂ | H | O | C(O)(4-OCH₃—C₆H₄) |
| A-169 | CH(CH₃)₂ | H | O | C(O)(4-Cl—C₆H₄) |
| A-170 | CH(CH₃)₂ | H | O | C(O)(4-Cl—C₆H₄) |
| A-171 | CH(CH₃)₂ | H | O | C(O)(4-pyridyl) |
| A-172 | CH(CH₃)₂ | H | O | C(O)(2-CH₂CH₃—C₆H₄) |
| A-173 | CH(CH₃)₂ | H | O | C(O)(2-CH₃—C₆H₄) |
| A-174 | CH(CH₃)₂ | H | O | C(O)(3-CH₃—C₆H₄) |
| A-175 | CH(CH₃)₂ | H | O | C(O)(4-CH₃—C₆H₄) |
| A-176 | CH(CH₃)₂ | CH(CH₃)₂ | O | H |
| A-177 | CH(CH₃)₂ | CH(CH₃)₂ | O | CH₃ |
| A-178 | CH(CH₃)₂ | CH(CH₃)₂ | O | CH₂CH₃ |
| A-179 | CH(CH₃)₂ | CH(CH₃)₂ | O | CH₂CH₂CH₃ |
| A-180 | CH(CH₃)₂ | CH(CH₃)₂ | O | CH(CH₃)₂ |
| A-181 | CH(CH₃)₂ | CH(CH₃)₂ | O | CH₂CH₂CH₂CH₃ |
| A-182 | CH(CH₃)₂ | CH(CH₃)₂ | O | CH(CH₃)CH₂CH₃ |
| A-183 | CH(CH₃)₂ | CH(CH₃)₂ | O | CH₂CH₂(CH₃)₂ |
| A-184 | CH(CH₃)₂ | CH(CH₃)₂ | O | C(CH₃)₃ |
| A-185 | CH(CH₃)₂ | CH(CH₃)₂ | O | C(O)CH₃ |
| A-186 | CH(CH₃)₂ | CH(CH₃)₂ | O | C(O)CH₂CH₃ |
| A-187 | CH(CH₃)₂ | CH(CH₃)₂ | O | C(O)CH₂CH₂CH₃ |
| A-188 | CH(CH₃)₂ | CH(CH₃)₂ | O | C(O)CH₂CH₂CH₂CH₃ |
| A-189 | CH(CH₃)₂ | CH(CH₃)₂ | O | C(O)CH(CH₂CH₃)₂ |
| A-190 | CH(CH₃)₂ | CH(CH₃)₂ | O | C(O)CH(CH₂F)₂ |
| A-191 | CH(CH₃)₂ | CH(CH₃)₂ | O | C(O)CH₂CH₂C₆H₅ |
| A-192 | CH(CH₃)₂ | CH(CH₃)₂ | O | C(O)C₆H₅ |
| A-193 | CH(CH₃)₂ | CH(CH₃)₂ | O | C(O)(4-OCH₃—C₆H₄) |
| A-194 | CH(CH₃)₂ | CH(CH₃)₂ | O | C(O)(4-Cl—C₆H₄) |
| A-195 | CH(CH₃)₂ | CH(CH₃)₂ | O | C(O)(4-Cl—C₆H₄) |
| A-196 | CH(CH₃)₂ | CH(CH₃)₂ | O | C(O)(4-pyridyl) |
| A-197 | CH(CH₃)₂ | CH(CH₃)₂ | O | C(O)(2-CH₂CH₃—C₆H₄) |
| A-198 | CH(CH₃)₂ | CH(CH₃)₂ | O | C(O)(2-CH₃—C₆H₄) |
| A-199 | CH(CH₃)₂ | CH(CH₃)₂ | O | C(O)(3-CH₃—C₆H₄) |
| A-200 | CH(CH₃)₂ | CH(CH₃)₂ | O | C(O)(4-CH₃—C₆H₄) |
| A-201 | C(CH₃)₃ | H | O | H |
| A-202 | C(CH₃)₃ | H | O | CH₃ |
| A-203 | C(CH₃)₃ | H | O | CH₂CH₃ |
| A-204 | C(CH₃)₃ | H | O | CH₂CH₂CH₃ |
| A-205 | C(CH₃)₃ | H | O | CH(CH₃)₂ |
| A-206 | C(CH₃)₃ | H | O | CH₂CH₂CH₂CH₃ |
| A-207 | C(CH₃)₃ | H | O | CH(CH₃)CH₂CH₃ |
| A-208 | C(CH₃)₃ | H | O | CH₂CH₂(CH₃)₂ |
| A-209 | C(CH₃)₃ | H | O | C(CH₃)₃ |
| A-210 | C(CH₃)₃ | H | O | C(O)CH₃ |
| A-211 | C(CH₃)₃ | H | O | C(O)CH₂CH₃ |
| A-212 | C(CH₃)₃ | H | O | C(O)CH₂CH₂CH₃ |
| A-213 | C(CH₃)₃ | H | O | C(O)CH₂CH₂CH₂CH₃ |
| A-214 | C(CH₃)₃ | H | O | C(O)CH(CH₂CH₃)₂ |
| A-215 | C(CH₃)₃ | H | O | C(O)CH(CH₂F)₂ |
| A-216 | C(CH₃)₃ | H | O | C(O)CH₂CH₂C₆H₅ |
| A-217 | C(CH₃)₃ | H | O | C(O)C₆H₅ |
| A-218 | C(CH₃)₃ | H | O | C(O)(4-OCH₃—C₆H₄) |
| A-219 | C(CH₃)₃ | H | O | C(O)(4-Cl—C₆H₄) |
| A-220 | C(CH₃)₃ | H | O | C(O)(4-Cl—C₆H₄) |
| A-221 | C(CH₃)₃ | H | O | C(O)(4-pyridyl) |
| A-222 | C(CH₃)₃ | H | O | C(O)(2-CH₂CH₃—C₆H₄) |
| A-223 | C(CH₃)₃ | H | O | C(O)(2-CH₃—C₆H₄) |
| A-224 | C(CH₃)₃ | H | O | C(O)(3-CH₃—C₆H₄) |
| A-225 | C(CH₃)₃ | H | O | C(O)(4-CH₃—C₆H₄) |
| A-226 | CH(CH₂CH₃)₂ | H | O | H |
| A-227 | CH(CH₂CH₃)₂ | H | O | CH₃ |
| A-228 | CH(CH₂CH₃)₂ | H | O | CH₂CH₃ |
| A-229 | CH(CH₂CH₃)₂ | H | O | CH₂CH₂CH₃ |
| A-230 | CH(CH₂CH₃)₂ | H | O | CH(CH₃)₂ |
| A-231 | CH(CH₂CH₃)₂ | H | O | CH₂CH₂CH₂CH₃ |
| A-232 | CH(CH₂CH₃)₂ | H | O | CH(CH₃)CH₂CH₃ |
| A-233 | CH(CH₂CH₃)₂ | H | O | CH₂CH₂(CH₃)₂ |
| A-234 | CH(CH₂CH₃)₂ | H | O | C(CH₃)₃ |
| A-235 | CH(CH₂CH₃)₂ | H | O | C(O)CH₃ |
| A-236 | CH(CH₂CH₃)₂ | H | O | C(O)CH₂CH₃ |
| A-237 | CH(CH₂CH₃)₂ | H | O | C(O)CH₂CH₂CH₃ |
| A-238 | CH(CH₂CH₃)₂ | H | O | C(O)CH₂CH₂CH₂CH₃ |
| A-239 | CH(CH₂CH₃)₂ | H | O | C(O)CH(CH₂CH₃)₂ |
| A-240 | CH(CH₂CH₃)₂ | H | O | C(O)CH(CH₂F)₂ |
| A-241 | CH(CH₂CH₃)₂ | H | O | C(O)CH₂CH₂C₆H₅ |
| A-242 | CH(CH₂CH₃)₂ | H | O | C(O)C₆H₅ |
| A-243 | CH(CH₂CH₃)₂ | H | O | C(O)(4-OCH₃—C₆H₄) |
| A-244 | CH(CH₂CH₃)₂ | H | O | C(O)(4-Cl—C₆H₄) |
| A-245 | CH(CH₂CH₃)₂ | H | O | C(O)(4-Cl—C₆H₄) |
| A-246 | CH(CH₂CH₃)₂ | H | O | C(O)(4-pyridyl) |
| A-247 | CH(CH₂CH₃)₂ | H | O | C(O)(2-CH₂CH₃—C₆H₄) |
| A-248 | CH(CH₂CH₃)₂ | H | O | C(O)(2-CH₃—C₆H₄) |
| A-249 | CH(CH₂CH₃)₂ | H | O | C(O)(3-CH₃—C₆H₄) |
| A-250 | CH(CH₂CH₃)₂ | H | O | C(O)(4-CH₃—C₆H₄) |
| A-251 | c-C₃H₅ | H | O | H |
| A-252 | c-C₃H₅ | H | O | CH₃ |
| A-253 | c-C₃H₅ | H | O | CH₂CH₃ |
| A-254 | c-C₃H₅ | H | O | CH₂CH₂CH₃ |
| A-255 | c-C₃H₅ | H | O | CH(CH₃)₂ |
| A-256 | c-C₃H₅ | H | O | CH₂CH₂CH₂CH₃ |
| A-257 | c-C₃H₅ | H | O | CH(CH₃)CH₂CH₃ |
| A-258 | c-C₃H₅ | H | O | CH₂CH₂(CH₃)₂ |
| A-259 | c-C₃H₅ | H | O | C(CH₃)₃ |
| A-260 | c-C₃H₅ | H | O | C(O)CH₃ |
| A-261 | c-C₃H₅ | H | O | C(O)CH₂CH₃ |
| A-262 | c-C₃H₅ | H | O | C(O)CH₂CH₂CH₃ |
| A-263 | c-C₃H₅ | H | O | C(O)CH₂CH₂CH₂CH₃ |
| A-264 | c-C₃H₅ | H | O | C(O)CH(CH₂CH₃)₂ |
| A-265 | c-C₃H₅ | H | O | C(O)CH(CH₂F)₂ |
| A-266 | c-C₃H₅ | H | O | C(O)CH₂CH₂C₆H₅ |
| A-267 | c-C₃H₅ | H | O | C(O)C₆H₅ |
| A-268 | c-C₃H₅ | H | O | C(O)(4-OCH₃—C₆H₄) |
| A-269 | c-C₃H₅ | H | O | C(O)(4-Cl—C₆H₄) |
| A-270 | c-C₃H₅ | H | O | C(O)(4-Cl—C₆H₄) |
| A-271 | c-C₃H₅ | H | O | C(O)(4-pyridyl) |
| A-272 | c-C₃H₅ | H | O | C(O)(2-CH₂CH₃—C₆H₄) |
| A-273 | c-C₃H₅ | H | O | C(O)(2-CH₃—C₆H₄) |
| A-274 | c-C₃H₅ | H | O | C(O)(3-CH₃—C₆H₄) |
| A-275 | c-C₃H₅ | H | O | C(O)(4-CH₃—C₆H₄) |
| A-276 | c-C₃H₅ | CH₃ | O | H |
| A-277 | c-C₃H₅ | CH₃ | O | CH₃ |
| A-278 | c-C₃H₅ | CH₃ | O | CH₂CH₃ |
| A-279 | c-C₃H₅ | CH₃ | O | CH₂CH₂CH₃ |
| A-280 | c-C₃H₅ | CH₃ | O | CH(CH₃)₂ |
| A-281 | c-C₃H₅ | CH₃ | O | CH₂CH₂CH₂CH₃ |
| A-282 | c-C₃H₅ | CH₃ | O | CH(CH₃)CH₂CH₃ |
| A-283 | c-C₃H₅ | CH₃ | O | CH₂CH₂(CH₃)₂ |
| A-284 | c-C₃H₅ | CH₃ | O | C(CH₃)₃ |
| A-285 | c-C₃H₅ | CH₃ | O | C(O)CH₃ |
| A-286 | c-C₃H₅ | CH₃ | O | C(O)CH₂CH₃ |
| A-287 | c-C₃H₅ | CH₃ | O | C(O)CH₂CH₂CH₃ |
| A-288 | c-C₃H₅ | CH₃ | O | C(O)CH₂CH₂CH₂CH₃ |
| A-289 | c-C₃H₅ | CH₃ | O | C(O)CH(CH₂CH₃)₂ |
| A-290 | c-C₃H₅ | CH₃ | O | C(O)CH(CH₂F)₂ |
| A-291 | c-C₃H₅ | CH₃ | O | C(O)CH₂CH₂C₆H₅ |
| A-292 | c-C₃H₅ | CH₃ | O | C(O)C₆H₅ |
| A-293 | c-C₃H₅ | CH₃ | O | C(O)(4-OCH₃—C₆H₄) |
| A-294 | c-C₃H₅ | CH₃ | O | C(O)(4-Cl—C₆H₄) |
| A-295 | c-C₃H₅ | CH₃ | O | C(O)(4-Cl—C₆H₄) |
| A-296 | c-C₃H₅ | CH₃ | O | C(O)(4-pyridyl) |
| A-297 | c-C₃H₅ | CH₃ | O | C(O)(2-CH₂CH₃—C₆H₄) |
| A-298 | c-C₃H₅ | CH₃ | O | C(O)(2-CH₃—C₆H₄) |
| A-299 | c-C₃H₅ | CH₃ | O | C(O)(3-CH₃—C₆H₄) |
| A-300 | c-C₃H₅ | CH₃ | O | C(O)(4-CH₃—C₆H₄) |
| A-301 | c-C₅H₉ | H | O | H |
| A-302 | c-C₅H₉ | H | O | CH₃ |
| A-303 | c-C₅H₉ | H | O | CH₂CH₃ |
| A-304 | c-C₅H₉ | H | O | CH₂CH₂CH₃ |
| A-305 | c-C₅H₉ | H | O | CH(CH₃)₂ |
| A-306 | c-C₅H₉ | H | O | CH₂CH₂CH₂CH₃ |
| A-307 | c-C₅H₉ | H | O | CH(CH₃)CH₂CH₃ |
| A-308 | c-C₅H₉ | H | O | CH₂CH₂(CH₃)₂ |
| A-309 | c-C₅H₉ | H | O | C(CH₃)₃ |
| A-310 | c-C₅H₉ | H | O | C(O)CH₃ |
| A-311 | c-C₅H₉ | H | O | C(O)CH₂CH₃ |
| A-312 | c-C₅H₉ | H | O | C(O)CH₂CH₂CH₃ |
| A-313 | c-C₅H₉ | H | O | C(O)CH₂CH₂CH₂CH₃ |
| A-314 | c-C₅H₉ | H | O | C(O)CH(CH₂CH₃)₂ |
| A-315 | c-C₅H₉ | H | O | C(O)CH(CH₂F)₂ |
| A-316 | c-C₅H₉ | H | O | C(O)CH₂CH₂C₆H₅ |
| A-317 | c-C₅H₉ | H | O | C(O)C₆H₅ |
| A-318 | c-C₅H₉ | H | O | C(O)(4-OCH₃—C₆H₄) |
| A-319 | c-C₅H₉ | H | O | C(O)(4-Cl—C₆H₄) |
| A-320 | c-C₅H₉ | H | O | C(O)(4-Cl—C₆H₄) |
| A-321 | c-C₅H₉ | H | O | C(O)(4-pyridyl) |
| A-322 | c-C₅H₉ | H | O | C(O)(2-CH₂CH₃—C₆H₄) |
| A-323 | c-C₅H₉ | H | O | C(O)(2-CH₃—C₆H₄) |
| A-324 | c-C₅H₉ | H | O | C(O)(3-CH₃—C₆H₄) |
| A-325 | c-C₅H₉ | H | O | C(O)(4-CH₃—C₆H₄) |
| A-326 | c-C₅H₉ | CH₃ | O | H |
| A-327 | c-C₅H₉ | CH₃ | O | CH₃ |
| A-328 | c-C₅H₉ | CH₃ | O | CH₂CH₃ |
| A-329 | c-C₅H₉ | CH₃ | O | CH₂CH₂CH₃ |
| A-330 | c-C₅H₉ | CH₃ | O | CH(CH₃)₂ |
| A-331 | c-C₅H₉ | CH₃ | O | CH₂CH₂CH₂CH₃ |
| A-332 | c-C₅H₉ | CH₃ | O | CH(CH₃)CH₂CH₃ |
| A-333 | c-C₅H₉ | CH₃ | O | CH₂CH₂(CH₃)₂ |
| A-334 | c-C₅H₉ | CH₃ | O | C(CH₃)₃ |
| A-335 | c-C₅H₉ | CH₃ | O | C(O)CH₃ |
| A-336 | c-C₅H₉ | CH₃ | O | C(O)CH₂CH₃ |
| A-337 | c-C₅H₉ | CH₃ | O | C(O)CH₂CH₂CH₃ |
| A-338 | c-C₅H₉ | CH₃ | O | C(O)CH₂CH₂CH₂CH₃ |
| A-339 | c-C₅H₉ | CH₃ | O | C(O)CH(CH₂CH₃)₂ |
| A-340 | c-C₅H₉ | CH₃ | O | C(O)CH(CH₂F)₂ |
| A-341 | c-C₅H₉ | CH₃ | O | C(O)CH₂CH₂C₆H₅ |
| A-342 | c-C₅H₉ | CH₃ | O | C(O)C₆H₅ |
| A-343 | c-C₅H₉ | CH₃ | O | C(O)(4-OCH₃—C₆H₄) |
| A-344 | c-C₅H₉ | CH₃ | O | C(O)(4-Cl—C₆H₄) |
| A-345 | c-C₅H₉ | CH₃ | O | C(O)(4-Cl—C₆H₄) |
| A-346 | c-C₅H₉ | CH₃ | O | C(O)(4-pyridyl) |
| A-347 | c-C₅H₉ | CH₃ | O | C(O)(2-CH₂CH₃—C₆H₄) |
| A-348 | c-C₅H₉ | CH₃ | O | C(O)(2-CH₃—C₆H₄) |
| A-349 | c-C₅H₉ | CH₃ | O | C(O)(3-CH₃—C₆H₄) |
| A-350 | c-C₅H₉ | CH₃ | O | C(O)(4—CH₃—C₆H₄) |
| A-351 | —CH₂CH₂— | | O | H |
| A-352 | CH₂CH₂ | | O | CH₃ |
| A-353 | CH₂CH₂ | | O | CH₂CH₃ |
| A-354 | CH₂CH₂ | | O | CH₂CH₂CH₃ |
| A-355 | CH₂CH₂ | | O | CH(CH₃)₂ |
| A-356 | CH₂CH₂ | | O | CH₂CH₂CH₂CH₃ |
| A-357 | CH₂CH₂ | | O | CH(CH₃)CH₂CH₃ |
| A-358 | CH₂CH₂ | | O | CH₂CH₂(CH₃)₂ |
| A-359 | CH₂CH₂ | | O | C(CH₃)₃ |
| A-360 | CH₂CH₂ | | O | C(O)CH₃ |
| A-361 | CH₂CH₂ | | O | C(O)CH₂CH₃ |
| A-362 | CH₂CH₂ | | O | C(O)CH₂CH₂CH₃ |
| A-363 | CH₂CH₂ | | O | C(O)CH₂CH₂CH₂CH₃ |
| A-364 | CH₂CH₂ | | O | C(O)CH(CH₂CH₃)₂ |
| A-365 | CH₂CH₂ | | O | C(O)CH(CH₂F)₂ |
| A-366 | CH₂CH₂ | | O | C(O)CH₂CH₂C₆H₅ |
| A-367 | CH₂CH₂ | | O | C(O)C₆H₅ |
| A-368 | CH₂CH₂ | | O | C(O)(4-OCH₃—C₆H₄) |
| A-369 | CH₂CH₂ | | O | C(O)(4-Cl—C₆H₄) |
| A-370 | CH₂CH₂ | | O | C(O)(4-Cl—C₆H₄) |
| A-371 | CH₂CH₂ | | O | C(O)(4-pyridyl) |
| A-372 | CH₂CH₂ | | O | C(O)(2-CH₂CH₃—C₆H₄) |
| A-373 | CH₂CH₂ | | O | C(O)(2-CH₃—C₆H₄) |
| A-374 | CH₂CH₂ | | O | C(O)(3-CH₃—C₆H₄) |
| A-375 | CH₂CH₂ | | O | C(O)(4-CH₃—C₆H₄) |
| A-376 | —CH₂CH₂CH₂CH₂— | | O | H |
| A-377 | —CH₂CH₂CH₂CH₂— | | O | CH₃ |
| A-378 | —CH₂CH₂CH₂CH₂— | | O | CH₂CH₃ |
| A-379 | —CH₂CH₂CH₂CH₂— | | O | CH₂CH₂CH₃ |
| A-380 | —CH₂CH₂CH₂CH₂— | | O | CH(CH₃)₂ |
| A-381 | —CH₂CH₂CH₂CH₂— | | O | CH₂CH₂CH₂CH₃ |
| A-382 | —CH₂CH₂CH₂CH₂— | | O | CH(CH₃)CH₂CH₃ |
| A-383 | —CH₂CH₂CH₂CH₂— | | O | CH₂CH₂(CH₃)₂ |
| A-384 | —CH₂CH₂CH₂CH₂— | | O | C(CH₃)₃ |
| A-385 | —CH₂CH₂CH₂CH₂— | | O | C(O)CH₃ |
| A-386 | —CH₂CH₂CH₂CH₂— | | O | C(O)CH₂CH₃ |
| A-387 | —CH₂CH₂CH₂CH₂— | | O | C(O)CH₂CH₂CH₃ |
| A-388 | —CH₂CH₂CH₂CH₂— | | O | C(O)CH₂CH₂CH₂CH₃ |
| A-389 | —CH₂CH₂CH₂CH₂— | | O | C(O)CH(CH₂CH₃)₂ |
| A-390 | —CH₂CH₂CH₂CH₂— | | O | C(O)CH(CH₂F)₂ |
| A-391 | —CH₂CH₂CH₂CH₂— | | O | C(O)CH₂CH₂C₆H₅ |
| A-392 | —CH₂CH₂CH₂CH₂— | | O | C(O)C₆H₅ |
| A-393 | —CH₂CH₂CH₂CH₂— | | O | C(O)(4-OCH₃—C₆H₄) |
| A-394 | —CH₂CH₂CH₂CH₂— | | O | C(O)(4-Cl—C₆H₄) |
| A-395 | —CH₂CH₂CH₂CH₂— | | O | C(O)(4-Cl—C₆H₄) |
| A-396 | —CH₂CH₂CH₂CH₂— | | O | C(O)(4-pyridyl) |
| A-397 | —CH₂CH₂CH₂CH₂— | | O | C(O)(2-CH₂CH₃—C₆H₄) |
| A-398 | —CH₂CH₂CH₂CH₂— | | O | C(O)(2-CH₃—C₆H₄) |
| A-399 | —CH₂CH₂CH₂CH₂— | | O | C(O)(3-CH₃—C₆H₄) |
| A-400 | —CH₂CH₂CH₂CH₂— | | O | C(O)(4-CH₃—C₆H₄) |
| A-401 | CH₃ | H | S | H |
| A-402 | CH₃ | H | S | CH₃ |
| A-403 | CH₃ | H | S | CH₂CH₃ |
| A-404 | CH₃ | H | S | CH₂CH₂CH₃ |
| A-405 | CH₃ | H | S | CH(CH₃)₂ |
| A-406 | CH₃ | H | S | CH₂CH₂CH₂CH₃ |
| A-407 | CH₃ | H | S | CH(CH₃)CH₂CH₃ |
| A-408 | CH₃ | H | S | CH₂CH₂(CH₃)₂ |
| A-409 | CH₃ | H | S | C(CH₃)₃ |
| A-410 | CH₃ | H | S | C(S)CH₃ |
| A-411 | CH₃ | H | S | C(S)CH₂CH₃ |
| A-412 | CH₃ | CH₃ | S | H |
| A-413 | CH₃ | CH₃ | S | CH₃ |
| A-414 | CH₃ | CH₃ | S | CH₂CH₃ |
| A-415 | CH₃ | CH₃ | S | CH₂CH₂CH₃ |
| A-416 | CH₃ | CH₃ | S | CH(CH₃)₂ |
| A-417 | CH₃ | CH₃ | S | CH₂CH₂CH₂CH₃ |
| A-418 | CH₃ | CH₃ | S | CH(CH₃)CH₂CH₃ |
| A-419 | CH₃ | CH₃ | S | CH₂CH₂(CH₃)₂ |
| A-420 | CH₃ | CH₃ | S | C(CH₃)₃ |
| A-421 | CH₃ | CH₃ | S | C(S)CH₃ |
| A-422 | CH₃ | CH₃ | S | C(S)CH₂CH₃ |
| A-423 | CH₂CH₃ | H | S | H |
| A-424 | CH₂CH₃ | H | S | CH₃ |
| A-425 | CH₂CH₃ | H | S | CH₂CH₃ |
| A-426 | CH₂CH₃ | H | S | CH₂CH₂CH₃ |
| A-427 | CH₂CH₃ | H | S | CH(CH₃)₂ |
| A-428 | CH₂CH₃ | H | S | CH₂CH₂CH₂CH₃ |
| A-429 | CH₂CH₃ | H | S | CH(CH₃)CH₂CH₃ |
| A-430 | CH₂CH₃ | H | S | CH₂CH₂(CH₃)₂ |
| A-431 | CH₂CH₃ | H | S | C(CH₃)₃ |
| A-432 | CH₂CH₃ | H | S | C(S)CH₃ |
| A-433 | CH₂CH₃ | H | S | C(S)CH₂CH₃ |
| A-434 | CH₂CH₃ | CH₂CH₃ | S | H |
| A-435 | CH₂CH₃ | CH₂CH₃ | S | CH₃ |
| A-436 | CH₂CH₃ | CH₂CH₃ | S | CH₂CH₃ |
| A-437 | CH₂CH₃ | CH₂CH₃ | S | CH₂CH₂CH₃ |
| A-438 | CH₂CH₃ | CH₂CH₃ | S | CH(CH₃)₂ |
| A-439 | CH₂CH₃ | CH₂CH₃ | S | CH₂CH₂CH₂CH₃ |
| A-440 | CH₂CH₃ | CH₂CH₃ | S | CH(CH₃)CH₂CH₃ |
| A-441 | CH₂CH₃ | CH₂CH₃ | S | CH₂CH₂(CH₃)₂ |
| A-442 | CH₂CH₃ | CH₂CH₃ | S | C(CH₃)₃ |
| A-443 | CH₂CH₃ | CH₂CH₃ | S | C(S)CH₃ |
| A-444 | CH₂CH₃ | CH₂CH₃ | S | C(S)CH₂CH₃ |
| A-445 | CH₂CH₂CH₃ | H | S | H |
| A-446 | CH₂CH₂CH₃ | H | S | CH₃ |
| A-447 | CH₂CH₂CH₃ | H | S | CH₂CH₃ |
| A-448 | CH₂CH₂CH₃ | H | S | CH₂CH₂CH₃ |
| A-449 | CH₂CH₂CH₃ | H | S | CH(CH₃)₂ |
| A-450 | CH₂CH₂CH₃ | H | S | CH₂CH₂CH₂CH₃ |
| A-451 | CH₂CH₂CH₃ | H | S | CH(CH₃)CH₂CH₃ |
| A-452 | CH₂CH₂CH₃ | H | S | CH₂CH₂(CH₃)₂ |
| A-453 | CH₂CH₂CH₃ | H | S | C(CH₃)₃ |
| A-454 | CH₂CH₂CH₃ | H | S | C(S)CH₃ |
| A-455 | CH₂CH₂CH₃ | H | S | C(S)CH₂CH₃ |
| A-456 | CH₂CH₂CH₃ | CH₂CH₂CH₃ | S | H |
| A-457 | CH₂CH₂CH₃ | CH₂CH₂CH₃ | S | CH₃ |
| A-458 | CH₂CH₂CH₃ | CH₂CH₂CH₃ | S | CH₂CH₃ |
| A-459 | CH₂CH₂CH₃ | CH₂CH₂CH₃ | S | CH₂CH₂CH₃ |
| A-460 | CH₂CH₂CH₃ | CH₂CH₂CH₃ | S | CH(CH₃)₂ |
| A-461 | CH₂CH₂CH₃ | CH₂CH₂CH₃ | S | CH₂CH₂CH₂CH₃ |
| A-462 | CH₂CH₂CH₃ | CH₂CH₂CH₃ | S | CH(CH₃)CH₂CH₃ |
| A-463 | CH₂CH₂CH₃ | CH₂CH₂CH₃ | S | CH₂CH₂(CH₃)₂ |
| A-464 | CH₂CH₂CH₃ | CH₂CH₂CH₃ | S | C(CH₃)₃ |
| A-465 | CH₂CH₂CH₃ | CH₂CH₂CH₃ | S | C(S)CH₃ |
| A-466 | CH₂CH₂CH₃ | CH₂CH₂CH₃ | S | C(S)CH₂CH₃ |
| A-467 | CH(CH₃)₂ | H | S | H |
| A-468 | CH(CH₃)₂ | H | S | CH₃ |
| A-469 | CH(CH₃)₂ | H | S | CH₂CH₃ |
| A-470 | CH(CH₃)₂ | H | S | CH₂CH₂CH₃ |
| A-471 | CH(CH₃)₂ | H | S | CH(CH₃)₂ |
| A-472 | CH(CH₃)₂ | H | S | CH₂CH₂CH₂CH₃ |
| A-473 | CH(CH₃)₂ | H | S | CH(CH₃)CH₂CH₃ |
| A-474 | CH(CH₃)₂ | H | S | CH₂CH₂(CH₃)₂ |
| A-475 | CH(CH₃)₂ | H | S | C(CH₃)₃ |
| A-476 | CH(CH₃)₂ | H | S | C(S)CH₃ |
| A-477 | CH(CH₃)₂ | H | S | C(S)CH₂CH₃ |
| A-478 | CH(CH₃)₂ | CH(CH₃)₂ | S | H |
| A-479 | CH(CH₃)₂ | CH(CH₃)₂ | S | CH₃ |
| A-480 | CH(CH₃)₂ | CH(CH₃)₂ | S | CH₂CH₃ |
| A-481 | CH(CH₃)₂ | CH(CH₃)₂ | S | CH₂CH₂CH₃ |
| A-482 | CH(CH₃)₂ | CH(CH₃)₂ | S | CH(CH₃)₂ |
| A-483 | CH(CH₃)₂ | CH(CH₃)₂ | S | CH₂CH₂CH₂CH₃ |
| A-484 | CH(CH₃)₂ | CH(CH₃)₂ | S | CH(CH₃)CH₂CH₃ |
| A-485 | CH(CH₃)₂ | CH(CH₃)₂ | S | CH₂CH₂(CH₃)₂ |
| A-486 | CH(CH₃)₂ | CH(CH₃)₂ | S | C(CH₃)₃ |
| A-487 | CH(CH₃)₂ | CH(CH₃)₂ | S | C(S)CH₃ |
| A-488 | CH(CH₃)₂ | CH(CH₃)₂ | S | C(S)CH₂CH₃ |
| A-489 | C(CH₃)₃ | H | S | H |
| A-490 | C(CH₃)₃ | H | S | CH₃ |
| A-491 | C(CH₃)₃ | H | S | CH₂CH₃ |
| A-492 | C(CH₃)₃ | H | S | CH₂CH₂CH₃ |
| A-493 | C(CH₃)₃ | H | S | CH(CH₃)₂ |
| A-494 | C(CH₃)₃ | H | S | CH₂CH₂CH₂CH₃ |
| A-495 | C(CH₃)₃ | H | S | CH(CH₃)CH₂CH₃ |
| A-496 | C(CH₃)₃ | H | S | CH₂CH₂(CH₃)₂ |
| A-497 | C(CH₃)₃ | H | S | C(CH₃)₃ |
| A-498 | C(CH₃)₃ | H | S | C(S)CH₃ |
| A-499 | C(CH₃)₃ | H | S | C(S)CH₂CH₃ |
| A-500 | CH(CH₂CH₃)₂ | H | S | H |
| A-501 | CH(CH₂CH₃)₂ | H | S | CH₃ |
| A-502 | CH(CH₂CH₃)₂ | H | S | CH₂CH₃ |
| A-503 | CH(CH₂CH₃)₂ | H | S | CH₂CH₂CH₃ |
| A-504 | CH(CH₂CH₃)₂ | H | S | CH(CH₃)₂ |
| A-505 | CH(CH₂CH₃)₂ | H | S | CH₂CH₂CH₂CH₃ |
| A-506 | CH(CH₂CH₃)₂ | H | S | CH(CH₃)CH₂CH₃ |
| A-507 | CH(CH₂CH₃)₂ | H | S | CH₂CH₂(CH₃)₂ |
| A-508 | CH(CH₂CH₃)₂ | H | S | C(CH₃)₃ |
| A-509 | CH(CH₂CH₃)₂ | H | S | C(S)CH₃ |
| A-510 | CH(CH₂CH₃)₂ | H | S | C(S)CH₂CH₃ |
| A-511 | c-C₃H₅ | H | S | H |
| A-512 | c-C₃H₅ | H | S | CH₃ |
| A-513 | c-C₃H₅ | H | S | CH₂CH₃ |
| A-514 | c-C₃H₅ | H | S | CH₂CH₂CH₃ |
| A-515 | c-C₃H₅ | H | S | CH(CH₃)₂ |
| A-516 | c-C₃H₅ | H | S | CH₂CH₂CH₂CH₃ |
| A-517 | c-C₃H₅ | H | S | CH(CH₃)CH₂CH₃ |
| A-518 | c-C₃H₅ | H | S | CH₂CH₂(CH₃)₂ |
| A-519 | c-C₃H₅ | H | S | C(CH₃)₃ |
| A-520 | c-C₃H₅ | H | S | C(S)CH₃ |
| A-521 | c-C₃H₅ | H | S | C(S)CH₂CH₃ |
| A-522 | c-C₃H₅ | CH₃ | S | H |
| A-523 | c-C₃H₅ | CH₃ | S | CH₃ |
| A-524 | c-C₃H₅ | CH₃ | S | CH₂CH₃ |
| A-525 | c-C₃H₅ | CH₃ | S | CH₂CH₂CH₃ |
| A-526 | c-C₃H₅ | CH₃ | S | CH(CH₃)₂ |
| A-527 | c-C₃H₅ | CH₃ | S | CH₂CH₂CH₂CH₃ |
| A-528 | c-C₃H₅ | CH₃ | S | CH(CH₃)CH₂CH₃ |
| A-529 | c-C₃H₅ | CH₃ | S | CH₂CH₂(CH₃)₂ |
| A-530 | c-C₃H₅ | CH₃ | S | C(CH₃)₃ |
| A-531 | c-C₃H₅ | CH₃ | S | C(S)CH₃ |
| A-532 | c-C₃H₅ | CH₃ | S | C(S)CH₂CH₃ |
| A-533 | c-C₅H₉ | H | S | H |
| A-534 | c-C₅H₉ | H | S | CH₃ |
| A-535 | c-C₅H₉ | H | S | CH₂CH₃ |
| A-536 | c-C₅H₉ | H | S | CH₂CH₂CH₃ |
| A-537 | c-C₅H₉ | H | S | CH(CH₃)₂ |
| A-538 | c-C₅H₉ | H | S | CH₂CH₂CH₂CH₃ |
| A-539 | c-C₅H₉ | H | S | CH(CH₃)CH₂CH₃ |
| A-540 | c-C₅H₉ | H | S | CH₂CH₂(CH₃)₂ |
| A-541 | c-C₅H₉ | H | S | C(CH₃)₃ |
| A-542 | c-C₅H₉ | H | S | C(S)CH₃ |
| A-543 | c-C₅H₉ | H | S | C(S)CH₂CH₃ |
| A-544 | c-C₅H₉ | CH₃ | S | H |
| A-545 | c-C₅H₉ | CH₃ | S | CH₃ |
| A-546 | c-C₅H₉ | CH₃ | S | CH₂CH₃ |
| A-547 | c-C₅H₉ | CH₃ | S | CH₂CH₂CH₃ |
| A-548 | c-C₅H₉ | CH₃ | S | CH(CH₃)₂ |
| A-549 | c-C₅H₉ | CH₃ | S | CH₂CH₂CH₂CH₃ |
| A-550 | c-C₅H₉ | CH₃ | S | CH(CH₃)CH₂CH₃ |
| A-551 | c-C₅H₉ | CH₃ | S | CH₂CH₂(CH₃)₂ |
| A-552 | c-C₅H₉ | CH₃ | S | C(CH₃)₃ |
| A-553 | c-C₅H₉ | CH₃ | S | C(S)CH₃ |
| A-554 | c-C₅H₉ | CH₃ | S | C(S)CH₂CH₃ |

| | | | |
|---|---|---|---|
| A-555 | —CH₂CH₂— | S | H |
| A-556 | CH₂CH₂ | S | CH₃ |
| A-557 | CH₂CH₂ | S | CH₂CH₃ |
| A-558 | CH₂CH₂ | S | CH₂CH₂CH₃ |
| A-559 | CH₂CH₂ | S | CH(CH₃)₂ |
| A-560 | CH₂CH₂ | S | CH₂CH₂CH₂CH₃ |
| A-561 | CH₂CH₂ | S | CH(CH₃)CH₂CH₃ |
| A-562 | CH₂CH₂ | S | CH₂CH₂(CH₃)₂ |
| A-563 | CH₂CH₂ | S | C(CH₃)₃ |
| A-564 | CH₂CH₂ | S | C(S)CH₃ |
| A-565 | CH₂CH₂ | S | C(S)CH₂CH₃ |
| A-566 | —CH₂CH₂CH₂CH₂— | S | H |
| A-567 | —CH₂CH₂CH₂CH₂— | S | CH₃ |
| A-568 | —CH₂CH₂CH₂CH₂— | S | CH₂CH₃ |
| A-569 | —CH₂CH₂CH₂CH₂— | S | CH₂CH₂CH₃ |
| A-570 | —CH₂CH₂CH₂CH₂— | S | CH(CH₃)₂ |
| A-571 | —CH₂CH₂CH₂CH₂— | S | CH₂CH₂CH₂CH₃ |
| A-572 | —CH₂CH₂CH₂CH₂— | S | CH(CH₃)CH₂CH₃ |
| A-573 | —CH₂CH₂CH₂CH₂— | S | CH₂CH₂(CH₃)₂ |
| A-574 | —CH₂CH₂CH₂CH₂— | S | C(CH₃)₃ |
| A-575 | —CH₂CH₂CH₂CH₂— | S | C(S)CH₃ |
| A-576 | —CH₂CH₂CH₂CH₂— | S | C(S)CH₂CH₃ |

TABLE B

| No. | R² | R³ | Y | Z |
|---|---|---|---|---|
| B-1 | CH₃ | H | O | OCH₃ |
| B-2 | CH₃ | H | O | OCH₂CH₃ |
| B-3 | CH₃ | H | O | OCH₂CH₂CH₃ |
| B-4 | CH₃ | H | O | OCH(CH₃)₂ |
| B-5 | CH₃ | H | O | OCH₂CH₂CH₂CH₃ |
| B-6 | CH₃ | H | O | OCH₂CH(CH₃)₂ |
| B-7 | CH₃ | H | O | OCH(CH₃)CH₂CH₃ |
| B-8 | CH₃ | H | O | OC(CH₃)₃ |
| B-9 | CH₃ | H | O | OCH₂CH₂CH₂CH₂CH₃ |
| B-10 | CH₃ | H | O | NH₂ |
| B-11 | CH₃ | H | O | NHCH₃ |
| B-12 | CH₃ | H | O | NHCH₂CH₃ |
| B-13 | CH₃ | H | O | NHCH₂CH₂CH₃ |
| B-14 | CH₃ | H | O | NHCH(CH₃)₂ |
| B-15 | CH₃ | H | O | NHCH₂CH₂CH₂CH₃ |
| B-16 | CH₃ | H | O | N(CH₃)₂ |
| B-17 | CH₃ | H | O | N(CH₃)CH₂CH₃ |
| B-18 | CH₃ | H | O | N(CH₃)CH₂CH₂CH₃ |
| B-19 | CH₃ | H | O | N(CH₃)CH(CH₃)₂ |
| B-20 | CH₃ | H | O | N(CH₃)CH₂CH₂CH₂CH₃ |
| B-21 | CH₃ | H | O | N(CH₂CH₃)₂ |
| B-22 | CH₃ | H | O | N(CH₂CH₃)CH₂CH₂CH₃ |
| B-23 | CH₃ | H | O | N(CH₃)CH(CH₃)₂ |
| B-24 | CH₃ | H | O | N(CH₃)CH₂CH₂CH₂CH₃ |
| B-25 | CH₃ | H | O | N(CH₂CH₂CH₃)₂ |
| B-26 | CH₃ | H | O | N(CH₂CH₂CH₃)CH(CH₃)₂ |
| B-27 | CH₃ | H | O | N(CH₂CH₂CH₃)CH₂CH₂CH₂CH₃ |
| B-28 | CH₃ | H | O | N(CH₂CH₂CH₂CH₃)₂ |
| B-29 | CH₃ | H | O | N(—CH₂CH₂OCH₂CH₂—) |
| B-30 | CH₃ | H | O | N(—CH₂CH₂N[CH₃]CH₂CH₂—) |
| B-31 | CH₃ | CH₃ | O | OCH₃ |
| B-32 | CH₃ | CH₃ | O | OCH₂CH₃ |
| B-33 | CH₃ | CH₃ | O | OCH₂CH₂CH₃ |
| B-34 | CH₃ | CH₃ | O | OCH(CH₃)₂ |
| B-35 | CH₃ | CH₃ | O | OCH₂CH₂CH₂CH₃ |
| B-36 | CH₃ | CH₃ | O | OCH₂CH(CH₃)₂ |
| B-37 | CH₃ | CH₃ | O | OCH(CH₃)CH₂CH₃ |
| B-38 | CH₃ | CH₃ | O | OC(CH₃)₃ |
| B-39 | CH₃ | CH₃ | O | OCH₂CH₂CH₂CH₂CH₃ |
| B-40 | CH₃ | CH₃ | O | NH₂ |
| B-41 | CH₃ | CH₃ | O | NHCH₃ |
| B-42 | CH₃ | CH₃ | O | NHCH₂CH₃ |
| B-43 | CH₃ | CH₃ | O | NHCH₂CH₂CH₃ |
| B-44 | CH₃ | CH₃ | O | NHCH(CH₃)₂ |
| B-45 | CH₃ | CH₃ | O | NHCH₂CH₂CH₂CH₃ |
| B-46 | CH₃ | CH₃ | O | N(CH₃)₂ |
| B-47 | CH₃ | CH₃ | O | N(CH₃)CH₂CH₃ |
| B-48 | CH₃ | CH₃ | O | N(CH₃)CH₂CH₂CH₃ |
| B-49 | CH₃ | CH₃ | O | N(CH₃)CH(CH₃)₂ |
| B-50 | CH₃ | CH₃ | O | N(CH₃)CH₂CH₂CH₂CH₃ |
| B-51 | CH₃ | CH₃ | O | N(CH₂CH₃)₂ |
| B-52 | CH₃ | CH₃ | O | N(CH₂CH₃)CH₂CH₂CH₃ |
| B-53 | CH₃ | CH₃ | O | N(CH₃)CH(CH₃)₂ |
| B-54 | CH₃ | CH₃ | O | N(CH₃)CH₂CH₂CH₂CH₃ |
| B-55 | CH₃ | CH₃ | O | N(CH₂CH₂CH₃)₂ |
| B-56 | CH₃ | CH₃ | O | N(CH₂CH₂CH₃)CH(CH₃)₂ |
| B-57 | CH₃ | CH₃ | O | N(CH₂CH₂CH₃)CH₂CH₂CH₂CH₃ |
| B-58 | CH₃ | CH₃ | O | N(CH₂CH₂CH₂CH₃)₂ |
| B-59 | CH₃ | CH₃ | O | N(—CH₂CH₂OCH₂CH₂—) |
| B-60 | CH₃ | CH₃ | O | N(—CH₂CH₂N[CH₃]CH₂CH₂—) |
| B-61 | CH₂CH₃ | H | O | OCH₃ |
| B-62 | CH₂CH₃ | H | O | OCH₂CH₃ |
| B-63 | CH₂CH₃ | H | O | OCH₂CH₂CH₃ |
| B-64 | CH₂CH₃ | H | O | OCH(CH₃)₂ |
| B-65 | CH₂CH₃ | H | O | OCH₂CH₂CH₂CH₃ |
| B-66 | CH₂CH₃ | H | O | OCH₂CH(CH₃)₂ |
| B-67 | CH₂CH₃ | H | O | OCH(CH₃)CH₂CH₃ |
| B-68 | CH₂CH₃ | H | O | OC(CH₃)₃ |
| B-69 | CH₂CH₃ | H | O | OCH₂CH₂CH₂CH₂CH₃ |
| B-70 | CH₂CH₃ | H | O | NH₂ |
| B-71 | CH₂CH₃ | H | O | NHCH₃ |
| B-72 | CH₂CH₃ | H | O | NHCH₂CH₃ |
| B-73 | CH₂CH₃ | H | O | NHCH₂CH₂CH₃ |
| B-74 | CH₂CH₃ | H | O | NHCH(CH₃)₂ |
| B-75 | CH₂CH₃ | H | O | NHCH₂CH₂CH₂CH₃ |
| B-76 | CH₂CH₃ | H | O | N(CH₃)₂ |
| B-77 | CH₂CH₃ | H | O | N(CH₃)CH₂CH₃ |
| B-78 | CH₂CH₃ | H | O | N(CH₃)CH₂CH₂CH₃ |
| B-79 | CH₂CH₃ | H | O | N(CH₃)CH(CH₃)₂ |
| B-80 | CH₂CH₃ | H | O | N(CH₃)CH₂CH₂CH₂CH₃ |
| B-81 | CH₂CH₃ | H | O | N(CH₂CH₃)₂ |
| B-82 | CH₂CH₃ | H | O | N(CH₂CH₃)CH₂CH₂CH₃ |
| B-83 | CH₂CH₃ | H | O | N(CH₃)CH(CH₃)₂ |
| B-84 | CH₂CH₃ | H | O | N(CH₃)CH₂CH₂CH₂CH₃ |
| B-85 | CH₂CH₃ | H | O | N(CH₂CH₂CH₃)₂ |
| B-86 | CH₂CH₃ | H | O | N(CH₂CH₂CH₃)CH(CH₃)₂ |
| B-87 | CH₂CH₃ | H | O | N(CH₂CH₂CH₃)CH₂CH₂CH₂CH₃ |
| B-88 | CH₂CH₃ | H | O | N(CH₂CH₂CH₂CH₃)₂ |
| B-89 | CH₂CH₃ | H | O | N(—CH₂CH₂OCH₂CH₂—) |
| B-90 | CH₂CH₃ | H | O | N(—CH₂CH₂N[CH₃]CH₂CH₂—) |
| B-91 | CH₂CH₂CH₃ | H | O | OCH₃ |
| B-92 | CH₂CH₂CH₃ | H | O | OCH₂CH₃ |
| B-93 | CH₂CH₂CH₃ | H | O | OCH₂CH₂CH₃ |
| B-94 | CH₂CH₂CH₃ | H | O | OCH(CH₃)₂ |
| B-95 | CH₂CH₂CH₃ | H | O | OCH₂CH₂CH₂CH₃ |
| B-96 | CH₂CH₂CH₃ | H | O | OCH₂CH(CH₃)₂ |
| B-97 | CH₂CH₂CH₃ | H | O | OCH(CH₃)CH₂CH₃ |
| B-98 | CH₂CH₂CH₃ | H | O | OC(CH₃)₃ |
| B-99 | CH₂CH₂CH₃ | H | O | OCH₂CH₂CH₂CH₂CH₃ |
| B-100 | CH₂CH₂CH₃ | H | O | NH₂ |
| B-101 | CH₂CH₂CH₃ | H | O | NHCH₃ |
| B-102 | CH₂CH₂CH₃ | H | O | NHCH₂CH₃ |
| B-103 | CH₂CH₂CH₃ | H | O | NHCH₂CH₂CH₃ |
| B-104 | CH₂CH₂CH₃ | H | O | NHCH(CH₃)₂ |
| B-105 | CH₂CH₂CH₃ | H | O | NHCH₂CH₂CH₂CH₃ |
| B-106 | CH₂CH₂CH₃ | H | O | N(CH₃)₂ |
| B-107 | CH₂CH₂CH₃ | H | O | N(CH₃)CH₂CH₃ |
| B-108 | CH₂CH₂CH₃ | H | O | N(CH₃)CH₂CH₂CH₃ |
| B-109 | CH₂CH₂CH₃ | H | O | N(CH₃)CH(CH₃)₂ |
| B-110 | CH₂CH₂CH₃ | H | O | N(CH₃)CH₂CH₂CH₂CH₃ |
| B-111 | CH₂CH₂CH₃ | H | O | N(CH₂CH₃)₂ |
| B-112 | CH₂CH₂CH₃ | H | O | N(CH₂CH₃)CH₂CH₂CH₃ |
| B-113 | CH₂CH₂CH₃ | H | O | N(CH₃)CH(CH₃)₂ |
| B-114 | CH₂CH₂CH₃ | H | O | N(CH₃)CH₂CH₂CH₂CH₃ |
| B-115 | CH₂CH₂CH₃ | H | O | N(CH₂CH₂CH₃)₂ |
| B-116 | CH₂CH₂CH₃ | H | O | N(CH₂CH₂CH₃)CH(CH₃)₂ |
| B-117 | CH₂CH₂CH₃ | H | O | N(CH₂CH₂CH₃)CH₂CH₂CH₂CH₃ |
| B-118 | CH₂CH₂CH₃ | H | O | N(CH₂CH₂CH₂CH₃)₂ |
| B-119 | CH₂CH₂CH₃ | H | O | N(—CH₂CH₂OCH₂CH₂—) |
| B-120 | CH₂CH₂CH₃ | H | O | N(—CH₂CH₂N[CH₃]CH₂CH₂—) |
| B-121 | CH(CH₃)₂ | H | O | OCH₃ |
| B-122 | CH(CH₃)₂ | H | O | OCH₂CH₃ |
| B-123 | CH(CH₃)₂ | H | O | OCH₂CH₂CH₃ |
| B-124 | CH(CH₃)₂ | H | O | OCH(CH₃)₂ |
| B-125 | CH(CH₃)₂ | H | O | OCH₂CH₂CH₂CH₃ |
| B-126 | CH(CH₃)₂ | H | O | OCH₂CH(CH₃)₂ |
| B-127 | CH(CH₃)₂ | H | O | OCH(CH₃)CH₂CH₃ |
| B-128 | CH(CH₃)₂ | H | O | OC(CH₃)₃ |
| B-129 | CH(CH₃)₂ | H | O | OCH₂CH₂CH₂CH₂CH₃ |
| B-130 | CH(CH₃)₂ | H | O | NH₂ |
| B-131 | CH(CH₃)₂ | H | O | NHCH₃ |
| B-132 | CH(CH₃)₂ | H | O | NHCH₂CH₃ |
| B-133 | CH(CH₃)₂ | H | O | NHCH₂CH₂CH₃ |
| B-134 | CH(CH₃)₂ | H | O | NHCH(CH₃)₂ |
| B-135 | CH(CH₃)₂ | H | O | NHCH₂CH₂CH₂CH₃ |
| B-136 | CH(CH₃)₂ | H | O | N(CH₃)₂ |
| B-137 | CH(CH₃)₂ | H | O | N(CH₃)CH₂CH₃ |
| B-138 | CH(CH₃)₂ | H | O | N(CH₃)CH₂CH₂CH₃ |
| B-139 | CH(CH₃)₂ | H | O | N(CH₃)CH(CH₃)₂ |
| B-140 | CH(CH₃)₂ | H | O | N(CH₃)CH₂CH₂CH₂CH₃ |
| B-141 | CH(CH₃)₂ | H | O | N(CH₂CH₃)₂ |
| B-142 | CH(CH₃)₂ | H | O | N(CH₂CH₃)CH₂CH₂CH₃ |
| B-143 | CH(CH₃)₂ | H | O | N(CH₃)CH(CH₃)₂ |
| B-144 | CH(CH₃)₂ | H | O | N(CH₃)CH₂CH₂CH₂CH₃ |
| B-145 | CH(CH₃)₂ | H | O | N(CH₂CH₂CH₃)₂ |
| B-146 | CH(CH₃)₂ | H | O | N(CH₂CH₂CH₃)CH(CH₃)₂ |
| B-147 | CH(CH₃)₂ | H | O | N(CH₂CH₂CH₃)CH₂CH₂CH₂CH₃ |
| B-148 | CH(CH₃)₂ | H | O | N(CH₂CH₂CH₂CH₃)₂ |
| B-149 | CH(CH₃)₂ | H | O | N(—CH₂CH₂OCH₂CH₂—) |
| B-150 | CH(CH₃)₂ | H | O | N(—CH₂CH₂N[CH₃]CH₂CH₂—) |
| B-151 | CH₂CH₂CH₂CH₃ | H | O | OCH₃ |
| B-152 | CH₂CH₂CH₂CH₃ | H | O | OCH₂CH₃ |
| B-153 | CH₂CH₂CH₂CH₃ | H | O | OCH₂CH₂CH₃ |
| B-154 | CH₂CH₂CH₂CH₃ | H | O | OCH(CH₃)₂ |
| B-155 | CH₂CH₂CH₂CH₃ | H | O | OCH₂CH₂CH₂CH₃ |
| B-156 | CH₂CH₂CH₂CH₃ | H | O | OCH₂CH(CH₃)₂ |
| B-157 | CH₂CH₂CH₂CH₃ | H | O | OCH(CH₃)CH₂CH₃ |
| B-158 | CH₂CH₂CH₂CH₃ | H | O | OC(CH₃)₃ |
| B-159 | CH₂CH₂CH₂CH₃ | H | O | OCH₂CH₂CH₂CH₂CH₃ |
| B-160 | CH₂CH₂CH₂CH₃ | H | O | NH₂ |
| B-161 | CH₂CH₂CH₂CH₃ | H | O | NHCH₃ |
| B-162 | CH₂CH₂CH₂CH₃ | H | O | NHCH₂CH₃ |
| B-163 | CH₂CH₂CH₂CH₃ | H | O | NHCH₂CH₂CH₃ |
| B-164 | CH₂CH₂CH₂CH₃ | H | O | NHCH(CH₃)₂ |
| B-165 | CH₂CH₂CH₂CH₃ | H | O | NHCH₂CH₂CH₂CH₃ |
| B-166 | CH₂CH₂CH₂CH₃ | H | O | N(CH₃)₂ |
| B-167 | CH₂CH₂CH₂CH₃ | H | O | N(CH₃)CH₂CH₃ |
| B-168 | CH₂CH₂CH₂CH₃ | H | O | N(CH₃)CH₂CH₂CH₃ |
| B-169 | CH₂CH₂CH₂CH₃ | H | O | N(CH₃)CH(CH₃)₂ |
| B-170 | CH₂CH₂CH₂CH₃ | H | O | N(CH₃)CH₂CH₂CH₂CH₃ |
| B-171 | CH₂CH₂CH₂CH₃ | H | O | N(CH₂CH₃)₂ |
| B-172 | CH₂CH₂CH₂CH₃ | H | O | N(CH₂CH₃)CH₂CH₂CH₃ |
| B-173 | CH₂CH₂CH₂CH₃ | H | O | N(CH₃)CH(CH₃)₂ |
| B-174 | CH₂CH₂CH₂CH₃ | H | O | N(CH₃)CH₂CH₂CH₂CH₃ |
| B-175 | CH₂CH₂CH₂CH₃ | H | O | N(CH₂CH₂CH₃)₂ |
| B-176 | CH₂CH₂CH₂CH₃ | H | O | N(CH₂CH₂CH₃)CH(CH₃)₂ |
| B-177 | CH₂CH₂CH₂CH₃ | H | O | N(CH₂CH₂CH₃)CH₂CH₂CH₂CH₃ |
| B-178 | CH₂CH₂CH₂CH₃ | H | O | N(CH₂CH₂CH₂CH₃)₂ |
| B-179 | CH₂CH₂CH₂CH₃ | H | O | N(—CH₂CH₂OCH₂CH₂—) |
| B-180 | CH₂CH₂CH₂CH₃ | H | O | N(—CH₂CH₂N[CH₃]CH₂CH₂—) |
| B-181 | C(CH₃)₃ | H | O | OCH₃ |
| B-182 | C(CH₃)₃ | H | O | OCH₂CH₃ |
| B-183 | C(CH₃)₃ | H | O | OCH₂CH₂CH₃ |
| B-184 | C(CH₃)₃ | H | O | OCH(CH₃)₂ |
| B-185 | C(CH₃)₃ | H | O | OCH₂CH₂CH₂CH₃ |
| B-186 | C(CH₃)₃ | H | O | OCH₂CH(CH₃)₂ |
| B-187 | C(CH₃)₃ | H | O | OCH(CH₃)CH₂CH₃ |
| B-188 | C(CH₃)₃ | H | O | OC(CH₃)₃ |
| B-189 | C(CH₃)₃ | H | O | OCH₂CH₂CH₂CH₂CH₃ |
| B-190 | C(CH₃)₃ | H | O | NH₂ |
| B-191 | C(CH₃)₃ | H | O | NHCH₃ |
| B-192 | C(CH₃)₃ | H | O | NHCH₂CH₃ |
| B-193 | C(CH₃)₃ | H | O | NHCH₂CH₂CH₃ |
| B-194 | C(CH₃)₃ | H | O | NHCH(CH₃)₂ |
| B-195 | C(CH₃)₃ | H | O | NHCH₂CH₂CH₂CH₃ |
| B-196 | C(CH₃)₃ | H | O | N(CH₃)₂ |
| B-197 | C(CH₃)₃ | H | O | N(CH₃)CH₂CH₃ |
| B-198 | C(CH₃)₃ | H | O | N(CH₃)CH₂CH₂CH₃ |
| B-199 | C(CH₃)₃ | H | O | N(CH₃)CH(CH₃)₂ |
| B-200 | C(CH₃)₃ | H | O | N(CH₃)CH₂CH₂CH₂CH₃ |
| B-201 | C(CH₃)₃ | H | O | N(CH₂CH₃)₂ |
| B-202 | C(CH₃)₃ | H | O | N(CH₂CH₃)CH₂CH₂CH₃ |
| B-203 | C(CH₃)₃ | H | O | N(CH₃)CH(CH₃)₂ |
| B-204 | C(CH₃)₃ | H | O | N(CH₃)CH₂CH₂CH₂CH₃ |
| B-205 | C(CH₃)₃ | H | O | N(CH₂CH₂CH₃)₂ |
| B-206 | C(CH₃)₃ | H | O | N(CH₂CH₂CH₃)CH(CH₃)₂ |
| B-207 | C(CH₃)₃ | H | O | N(CH₂CH₂CH₃)CH₂CH₂CH₂CH₃ |
| B-208 | C(CH₃)₃ | H | O | N(CH₂CH₂CH₂CH₃)₂ |
| B-209 | C(CH₃)₃ | H | O | N(—CH₂CH₂OCH₂CH₂—) |
| B-210 | C(CH₃)₃ | H | O | N(—CH₂CH₂N[CH₃]CH₂CH₂—) |
| B-211 | C(CH₃)CH₂CH₃ | H | O | OCH₃ |
| B-212 | C(CH₃)CH₂CH₃ | H | O | OCH₂CH₃ |
| B-213 | C(CH₃)CH₂CH₃ | H | O | OCH₂CH₂CH₃ |
| B-214 | C(CH₃)CH₂CH₃ | H | O | OCH(CH₃)₂ |
| B-215 | C(CH₃)CH₂CH₃ | H | O | OCH₂CH₂CH₂CH₃ |
| B-216 | C(CH₃)CH₂CH₃ | H | O | OCH₂CH(CH₃)₂ |
| B-217 | C(CH₃)CH₂CH₃ | H | O | OCH(CH₃)CH₂CH₃ |
| B-218 | C(CH₃)CH₂CH₃ | H | O | OC(CH₃)₃ |
| B-219 | C(CH₃)CH₂CH₃ | H | O | OCH₂CH₂CH₂CH₂CH₃ |
| B-220 | C(CH₃)CH₂CH₃ | H | O | NH₂ |
| B-221 | C(CH₃)CH₂CH₃ | H | O | NHCH₃ |
| B-222 | C(CH₃)CH₂CH₃ | H | O | NHCH₂CH₃ |
| B-223 | C(CH₃)CH₂CH₃ | H | O | NHCH₂CH₂CH₃ |
| B-224 | C(CH₃)CH₂CH₃ | H | O | NHCH(CH₃)₂ |
| B-225 | C(CH₃)CH₂CH₃ | H | O | NHCH₂CH₂CH₂CH₃ |
| B-226 | C(CH₃)CH₂CH₃ | H | O | N(CH₃)₂ |
| B-227 | C(CH₃)CH₂CH₃ | H | O | N(CH₃)CH₂CH₃ |
| B-228 | C(CH₃)CH₂CH₃ | H | O | N(CH₃)CH₂CH₂CH₃ |
| B-229 | C(CH₃)CH₂CH₃ | H | O | N(CH₃)CH(CH₃)₂ |
| B-230 | C(CH₃)CH₂CH₃ | H | O | N(CH₃)CH₂CH₂CH₂CH₃ |
| B-231 | C(CH₃)CH₂CH₃ | H | O | N(CH₂CH₃)₂ |
| B-232 | C(CH₃)CH₂CH₃ | H | O | N(CH₂CH₃)CH₂CH₂CH₃ |
| B-233 | C(CH₃)CH₂CH₃ | H | O | N(CH₃)CH(CH₃)₂ |
| B-234 | C(CH₃)CH₂CH₃ | H | O | N(CH₃)CH₂CH₂CH₂CH₃ |
| B-235 | C(CH₃)CH₂CH₃ | H | O | N(CH₂CH₂CH₃)₂ |
| B-236 | C(CH₃)CH₂CH₃ | H | O | N(CH₂CH₂CH₃)CH(CH₃)₂ |
| B-237 | C(CH₃)CH₂CH₃ | H | O | N(CH₂CH₂CH₃)CH₂CH₂CH₂CH₃ |
| B-238 | C(CH₃)CH₂CH₃ | H | O | N(CH₂CH₂CH₂CH₃)₂ |
| B-239 | C(CH₃)CH₂CH₃ | H | O | N(—CH₂CH₂OCH₂CH₂—) |
| B-240 | C(CH₃)CH₂CH₃ | H | O | N(—CH₂CH₂N[CH₃]CH₂CH₂—) |
| B-241 | CH₂CH(CH₃)₂ | H | O | OCH₃ |
| B-242 | CH₂CH(CH₃)₂ | H | O | OCH₂CH₃ |
| B-243 | CH₂CH(CH₃)₂ | H | O | OCH₂CH₂CH₃ |
| B-244 | CH₂CH(CH₃)₂ | H | O | OCH(CH₃)₂ |
| B-245 | CH₂CH(CH₃)₂ | H | O | OCH₂CH₂CH₂CH₃ |
| B-246 | CH₂CH(CH₃)₂ | H | O | OCH₂CH(CH₃)₂ |
| B-247 | CH₂CH(CH₃)₂ | H | O | OCH(CH₃)CH₂CH₃ |
| B-248 | CH₂CH(CH₃)₂ | H | O | OC(CH₃)₃ |
| B-249 | CH₂CH(CH₃)₂ | H | O | OCH₂CH₂CH₂CH₂CH₃ |
| B-250 | CH₂CH(CH₃)₂ | H | O | NH₂ |
| B-251 | CH₂CH(CH₃)₂ | H | O | NHCH₃ |
| B-252 | CH₂CH(CH₃)₂ | H | O | NHCH₂CH₃ |
| B-253 | CH₂CH(CH₃)₂ | H | O | NHCH₂CH₂CH₃ |
| B-254 | CH₂CH(CH₃)₂ | H | O | NHCH(CH₃)₂ |
| B-255 | CH₂CH(CH₃)₂ | H | O | NHCH₂CH₂CH₂CH₃ |
| B-256 | CH₂CH(CH₃)₂ | H | O | N(CH₃)₂ |
| B-257 | CH₂CH(CH₃)₂ | H | O | N(CH₃)CH₂CH₃ |
| B-258 | CH₂CH(CH₃)₂ | H | O | N(CH₃)CH₂CH₂CH₃ |
| B-259 | CH₂CH(CH₃)₂ | H | O | N(CH₃)CH(CH₃)₂ |
| B-260 | CH₂CH(CH₃)₂ | H | O | N(CH₃)CH₂CH₂CH₂CH₃ |
| B-261 | CH₂CH(CH₃)₂ | H | O | N(CH₂CH₃)₂ |
| B-262 | CH₂CH(CH₃)₂ | H | O | N(CH₂CH₃)CH₂CH₂CH₃ |
| B-263 | CH₂CH(CH₃)₂ | H | O | N(CH₃)CH(CH₃)₂ |
| B-264 | CH₂CH(CH₃)₂ | H | O | N(CH₃)CH₂CH₂CH₂CH₃ |
| B-265 | CH₂CH(CH₃)₂ | H | O | N(CH₂CH₂CH₃)₂ |
| B-266 | CH₂CH(CH₃)₂ | H | O | N(CH₂CH₂CH₃)CH(CH₃)₂ |
| B-267 | CH₂CH(CH₃)₂ | H | O | N(CH₂CH₂CH₃)CH₂CH₂CH₂CH₃ |
| B-268 | CH₂CH(CH₃)₂ | H | O | N(CH₂CH₂CH₂CH₃)₂ |
| B-269 | CH₂CH(CH₃)₂ | H | O | N(—CH₂CH₂OCH₂CH₂—) |
| B-270 | CH₂CH(CH₃)₂ | H | O | N(—CH₂CH₂N[CH₃]CH₂CH₂—) |

The compounds I are suitable as fungicides. They are distinguished by an outstanding effectiveness against a broad spectrum of phytopathogenic fungi, especially from the classes of the Ascomycetes, Deuteromycetes, Oomycetes and Basidiomycetes. Some are systemically effective and they can be used in plant protection as foliar fungicides, as fungicides for seed dressing and as soil fungicides.

The present invention further relates to the pharmaceutical use of the compounds of the formula I according to the invention and/or the pharmaceutically acceptable salts thereof, in particular to their use for treating tumors in mammals such as humans for example.

They are particularly important in plant protection in the control of a multitude of fungi on various cultivated plants, such as wheat, rye, barley, oats, rice, maize, grass, bananas, cotton, soya, coffee, sugar cane, vines, fruits and ornamental plants, and vegetables, such as cucumbers, beans, tomatoes, potatoes and cucurbits, and on the seeds of these plants.

They are especially suitable for controlling the following plant diseases:
Alternaria species on vegetables, rapeseed, sugar beet and fruit and rice,
Aphanomyces species on sugar beet and vegetables,
Bipolaris and Drechslera species on corn, cereals, rice and lawns,
Blumeria gramihis (powdery mildew) on cereals,
Botrytis cinerea (gray mold) on strawberries, vegetables, ornamental plants and grapevines,
Bremia lactucae on lettuce,
Cercospora species on corn, soybean, rice and sugar beet,
Cochliobolus species on corn, cereals, rice (e.g., Cochliobolus sativus on cereals, Cochliobolus miyabeanus on rice),
Colletotricum species on soybean and cotton
Drechslera species on cereals and corn,
Exserohilum species on corn,
Erysiphe cichoracearum and Sphaerotheca fuliginea on cucurbits,
Fusarium and Verticillium species on various plants,
Gaeumanomyces graminis on cereals,
Gibberella species on cereals and rice (e.g., Gibberella fujikuroi on rice),
Grainstaining complex on rice,
Helminthosporium species on corn and rice,
Michrodochium nivale on cereals,
Mycosphaerella species on cereals, bananas and peanuts,
Phakopsara pachyrhizi and Phakopsara meibomiae on soybean,
Phomopsis species on soybean and sunflowers,
Phytophthora infestans on potatoes and tomatoes,
Plasmopara viticola on grapevines,
Podosphaera leucotricha on apples,
Pseudocercosporella herpotrichoides on cereals,
Pseudoperonospora species on hops and cucurbits,
Puccinia species on cereals and corn,
Pyrenophora species on cereals,
Pyricularia oryzae, Corticium sasakii, Sarocladium oryzae, S. attenuatum, Entyloma oryzae on rice,
Pyricularia grisea on lawns and cereals
Pythium spp. on lawns, rice, corn, cotton, rapeseed, sunflowers, sugar beet, vegetables and other plants,
Rhizoctonia species on cotton, rice, potatoes, lawns, corn, rapeseed, potatoes, sugar beet, vegetables and other plants,
Sclerotinia species on rapeseed and sunflowers,
Septoria tritici and Stagonospora nodorum on wheat,
Erysiphe (syn. Uncinula) necatoron grapevines,
Setospaeria species on corn and lawns,

Sphacelotheca reilinia on corn,
Thievaliopsis species on soybean and cotton,
Tilletia species on cereals,
Ustilago species on cereals, corn and sugar beet, and
Venturia species (scab) on apples and pears.

The compounds I are also suitable for controlling harmful fungi, such as Paecilomyces variotii, in the protection of materials (e.g. wood, paper, paint dispersions, fibers or fabrics) and in the protection of stored products.

The compounds I are employed by treating the fungi or the plants, seeds, materials or soil to be protected from fungal attack with a fungicidally effective amount of the active compounds. The application can be carried out both before and after the infection of the materials, plants or seeds by the fungi.

The fungicidal compositions generally comprise between 0.1 and 95%, preferably between 0.5 and 90%, by weight of active compound.

When employed in plant protection, the amounts applied are, depending on the kind of effect desired, between 0.01 and 2.0 kg of active compound per ha.

In seed treatment, for example dusting, coating or impregnation of seed, amounts of active compound of from 1 to 1000 g/100 kg of seed, preferably from 1 to 200 g/100 kg, in particular from 5 to 100 g/100 kg are generally used.

When used in the protection of materials or stored products, the amount of active compound applied depends on the kind of application area and on the desired effect. Amounts customarily applied in the protection of materials are, for example, 0.001 g to 2 kg, preferably 0.005 g to 1 kg, of active compound per cubic meter of treated material.

The compounds of the formula I can be present in various crystalline forms which likewise form part of the subject matter of the present invention.

The compounds I can be converted into the customary formulations, for example solutions, emulsions, suspensions, dusts, powders, pastes and granules. The application form depends on the particular purpose; in each case, it should ensure a fine and uniform distribution of the compound according to the invention.

Formulations for seed treatment may further comprise binders and/or gelants and if appropriate dyes.

Binders can be added to increase the adhesion of the active compounds to the seed after the treatment. Suitable binders are for example EO/PO block copolymer surfactants, but also polyvinyl alcohols, polyvinylpyrrolidones, polyacrylates, polymethacrylates, polybutenes, polyisobutylenes, polystyrenes, polyethyleneamines, polyethyleneamides, polyethyleneimines (Lupasol®), Polymin®), polyethers, polyurethanes, polyvinyl acetates, Tylose and copolymers of these polymers. A suitable gelant is for example carrageen (Satiagel®)).

The formulations are prepared in a known manner, for example by extending the active compound with solvents and/or carriers, if appropriate using emulsifiers and dispersants. Solvents/auxiliaries which are suitable are essentially:
water, aromatic solvents (for example Solvesso products, xylene), paraffins (for example mineral oil fractions), alcohols (for example methanol, butanol, pentanol, benzyl alcohol), ketones (for example cyclohexanone, gamma-butyrolactone), pyrrolidones (NMP, NOP), acetates (glycol diacetate), glycols, fatty acid dimethylamides, fatty acids and fatty acid esters. In principle, solvent mixtures may also be used,
carriers such as ground natural minerals (for example kaolins, clays, talc, chalk) and ground synthetic minerals (for example highly disperse silica, silicates); emulsifiers such as nonionic and anionic emulsifiers (for example polyoxyethylene fatty alcohol ethers, alkylsulfonates and arylsulfonates) and dispersants such as lignosulfite waste liquors and methylcellulose.

Suitable surfactants are alkali metal, alkaline earth metal and ammonium salts of lignosulfonic acid, naphthalenesulfonic acid, phenolsulfonic acid, dibutylnaphthalenesulfonic acid, alkylarylsulfonates, alkyl sulfates, alkylsulfonates, fatty alcohol sulfates, fatty acids and sulfated fatty alcohol glycol ethers, furthermore condensates of sulfonated naphthalene and naphthalene derivatives with formaldehyde, condensates of naphthalene or of naphthalenesulfonic acid with phenol and formaldehyde, polyoxyethylene octylphenol ether, ethoxylated isooctylphenol, octylphenol, nonylphenol, alkylphenol polyglycol ethers, tributylphenyl polyglycol ether, tristearylphenyl polyglycol ether, alkylaryl polyether alcohols, alcohol and fatty alcohol/ethylene oxide condensates, ethoxylated castor oil, polyoxyethylene alkyl ethers, ethoxylated polyoxypropylene, lauryl alcohol polyglycol ether acetal, sorbitol esters, lignosulfite waste liquors and methylcellulose.

Suitable for the preparation of directly sprayable solutions, emulsions, pastes or oil dispersions are mineral oil fractions of medium to high boiling point, such as kerosene or diesel oil, furthermore coal tar oils and oils of vegetable or animal origin, aliphatic, cyclic and aromatic hydrocarbons, for example toluene, xylene, paraffin, tetrahydronaphthalene, alkylated naphthalenes or their derivatives, methanol, ethanol, propanol, butanol, cyclohexanol, cyclohexanone, isophorone, strongly polar solvents, for example dimethyl sulfoxide, N-methylpyrrolidone and water.

Powders, materials for spreading and dustable products can be prepared by mixing or concomitantly grinding the active substances with a solid carrier.

Granules, for example coated granules, impregnated granules and homogeneous granules, can be prepared by binding the active compounds to solid carriers. Examples of solid carriers are mineral earths such as silica gels, silicates, talc, kaolin, attaclay, limestone, lime, chalk, bole, loess, clay, dolomite, diatomaceous earth, calcium sulfate, magnesium sulfate, magnesium oxide, ground synthetic materials, fertilizers, such as, for example, ammonium sulfate, ammonium phosphate, ammonium nitrate, ureas, and products of vegetable origin, such as cereal meal, tree bark meal, wood meal and nutshell meal, cellulose powders and other solid carriers.

In general, the formulations comprise from 0.01 to 95% by weight, preferably from 0.1 to 90% by weight, of the active compound. The active compounds are employed in a purity of from 90% to 100%, preferably 95% to 100% (according to NMR spectrum).

For seed treatment, the formulations in question, when diluted from two- to ten-fold, produce active compound concentrations in the range from 0.01% to 60% by weight and preferably in the range from 0.1% to 40% by weight in the ready-to-use preparations.

The following are examples of formulations: 1. Products for dilution with water

A Water-Soluble Concentrates (SL, LS)

10 parts by weight of a compound according to the invention are dissolved with 90 parts by weight of water or with a water-soluble solvent. As an alternative, wetters or other auxiliaries are added. The active compound dissolves upon dilution with water. This gives a formulation having an active compound content of 10% by weight.

B Dispersible Concentrates (DC)

20 parts by weight of a compound according to the invention are dissolved in 70 parts by weight of cyclohexanone with addition of 10 parts by weight of a dispersant, for example polyvinylpyrrolidone. Dilution with water gives a dispersion. The active compound concentration is 20% by weight

C Emulsifiable Concentrates (EC)

15 parts by weight of a compound according to the invention are dissolved in 75 parts by weight of xylene with addition of calcium dodecylbenzenesulfonate and castor oil ethoxylate (in each case 5%). Dilution with water gives an emulsion. The formulation has an active compound content of 15% by weight.

D Emulsions (EW, EO, ES)

25 parts by weight of a compound according to the invention are dissolved in 35 parts by weight of xylene with addition of calcium dodecylbenzenesulfonate and castor oil ethoxylate (in each case 5 parts by weight). This mixture is added to 30 parts by weight of water by means of an emulsifying machine (e.g. Ultraturrax) and made into a homogeneous emulsion. Dilution with water gives an emulsion. The formulation has an active compound content of 25% by weight.

E Suspensions (SC, OD, FS)

In an agitated ball mill, 20 parts by weight of a compound according to the invention are comminuted with addition of 10 parts by weight of dispersants and wetters and 70 parts by weight of water or an organic solvent to give a fine active compound suspension. Dilution with water gives a stable suspension of the active compound. The active compound content in the formulation is 20% by weight.

F Water-Dispersible Granules and Water-Soluble Granules (WG, SG)

50 parts by weight of a compound according to the invention are ground finely with addition of 50 parts by weight of dispersants and wetters and made into water-dispersible or water-soluble granules by means of technical appliances (for example extrusion, spray tower, fluidized bed). Dilution with water gives a stable dispersion or solution of the active compound. The formulation has an active compound content of 50% by weight.

G Water-Dispersible Powders and Water-Soluble Powders (WP, SP, SS, WS)

75 parts by weight of a compound according to the invention are ground in a rotor-stator mill with addition of 25 parts by weight of dispersants, wetters and silica gel. Dilution with water gives a stable dispersion or solution of the active compound. The active compound content of the formulation is 75% by weight.

H Gel Formulations

20 parts by weight of a compound according to the invention, 10 parts by weight of dispersant, 1 part by weight of gelant and 70 parts by weight of water or of an organic solvent are ball milled to form a fine suspension. Dilution with water gives a stable suspension having an active compound content of 20% by weight.

2. Products to be Applied Undiluted

I Dustable Powders (DP, DS)

5 parts by weight of a compound according to the invention are ground finely and mixed intimately with 95 parts by weight of finely divided kaolin. This gives a dustable product with an active compound content of 5% by weight.

J Granules (GR, FG, GG, MG)

0.5 part by weight of a compound according to the invention is ground finely and associated with 99.5 parts by weight of carriers. Current methods are extrusion, spray-drying or the fluidized bed. This gives granules with an active compound content of 0.5% by weight to be applied undiluted.

K ULV Solutions (UL)

10 parts by weight of a compound according to the invention are dissolved in 90 parts by weight of an organic solvent, for example xylene. This gives a product with an active compound content of 10% by weight to be applied undiluted.

Seed treatment typically utilizes water-soluble concentrates (LS), suspensions (FS), dusts (DS), water-dispersible and water-soluble powders (WS, SS), emulsions (ES), emulsifiable concentrates (EC) and gel formulations (GF). These formulations can be applied neat or preferably diluted to the seed. The application can take place prior to sowing.

Preference is given to using FS formulations for seed treatment. Such formulations typically comprise from 1 to 800 g/l of active compound, from 1 to 200 g/l of surfactants, from 0 to 200 g/l of antifreeze, from 0 to 400 g/l of binder, from 0 to 200 g/l of dyes and solvent, preferably water.

The active compounds can be used as such, in the form of their formulations or the use forms prepared therefrom, for example in the form of directly sprayable solutions, powders, suspensions or dispersions, emulsions, oil dispersions, pastes, dustable products, materials for spreading, or granules, by means of spraying, atomizing, dusting, spreading or pouring. The use forms depend entirely on the intended purposes; the intention is to ensure in each case the finest possible distribution of the active compounds according to the invention.

Aqueous use forms can be prepared from emulsion concentrates, pastes or wettable powders (sprayable powders, oil dispersions) by adding water. To prepare emulsions, pastes or oil dispersions, the substances, as such or dissolved in an oil or solvent, can be homogenized in water by means of a wetting agent, tackifier, dispersant or emulsifier. Alternatively, it is possible to prepare concentrates composed of active substance, wetter, tackifier, dispersant or emulsifier and, if appropriate, solvent or oil, and such concentrates are suitable for dilution with water.

The active compound concentrations in the ready-to-use preparations can be varied within relatively wide ranges. In general, they are from 0.0001 to 10%, preferably from 0.01 to 1%.

The active compounds may also be used successfully in the ultra-low-volume process (ULV), by which it is possible to apply formulations comprising over 95% by weight of active compound, or even to apply the active compound without additives.

Various types of oils, wetters, adjuvants, herbicides, fungicides, other pesticides, or bactericides may be added to the active compounds, if appropriate not until immediately prior to use (tank mix). These agents can be admixed with the agents according to the invention in a weight ratio of 1:10 to 10:1.

The compositions according to the invention can, in the use form as fungicides, also be present together with other active compounds, e.g. with herbicides, insecticides, growth regulators, fungicides or else with fertilizers. Mixing the compounds I or the compositions comprising them in the application form as fungicides with further active compounds, in particular with other fungicides makes it possible in many cases to broaden the activity spectrum or prevent developments of resistance. Synergistic effects are obtained in many cases.

The following list of fungicides, in conjunction with which the compounds according to the invention can be used, is intended to illustrate the possible combinations but does not limit them:
acylalanines, such as benalaxyl, metalaxyl, ofurace or oxadixyl,
amine derivatives, such as aldimorph, dodine, dodemorph, fenpropimorph, fenpropidin, guazatine, iminoctadine, spiroxamine or tridemorph,
anilinopyrimidines, such as pyrimethanil, mepanipyrim or cyprodinyl,
antibiotics, such as cycloheximide, griseofulvin, kasugamycin, natamycin, polyoxin or streptomycin,
azoles, such as bitertanol, bromoconazole, cyproconazole, difenoconazole, dinitroconazole, enilconazole, epoxiconazole, fenbuconazole, fluquinconazole, flusilazole, flutriafole hexaconazole, imazalil, metconazole, myclobutanil, penconazole, propiconazole, prochloraz, prothioconazole, tebuconazole, triadimefon, triadimenol, triflumizole or triticonazole,
dicarboximides, such as iprodione, myclozolin, procymidone or vinclozolin,
dithiocarbamates, such as ferbam, nabam, maneb, mancozeb, metam, metiram, propineb, polycarbamate, thiram, ziram or zineb,
heterocyclic compounds, such as anilazine, benomyl, boscalid, carbendazim, carboxin, oxycarboxin, cyazofamid, dazomet, dithianon, famoxadone, fenamidone, fenarimol, fuberidazole, flutolanil, furametpyr, isoprothiolane, mepronil, nuarimol, penthiopyrad, probenazole, proquinazid, pyrifenox, pyroquilon, quinoxyfen, silthiofam, thiabendazole, thifluzamide, thiophanate-methyl, tiadinil, tricyclazole or triforine, N-(4′-bromobiphenyl-2-yl)-4-difluoromethyl-2-methylthiazole-5-carboxamide, N-(4′-trifluoromethylbiphenyl-2-yl)-4-difluoromethyl-2-methylthiazole-5-carboxamide, N-(4′-chloro-3′-fluorobiphenyl-2-yl)-4-difluoromethyl-2-methylthiazole-5-carboxamide, N-(3′,4′-dichloro-4-fluorobiphenyl-2-yl)-3-difluoromethyl-1-methylpyrazole-4-carboxamide, N-(2-cyanophenyl)-3,4-dichloroisothiazole-5-carboxamide, 5-chloro-7-(4-methylpiperidin-1-yl)-6-(2,4,6-trifluorophenyl)-[1,2,4]triazolo[1,5-a]pyrimidine, 2-butoxy-6-iodo-3-propylchromen-4-one, N-dimethyl-3-(3-bromo-6-fluoro-2-methylindole-1-sulfonyl)-[1,2,4]triazole-1-sulfonamide,
copper fungicides, such as Bordeaux mixture, copper acetate, copper oxychloride or basic copper sulfate,
nitrophenyl derivatives, such as binapacryl, dinocap, dinobuton or nitrophthal-isopropyl,
phenylpyrroles, such as fenpiclonil or fludioxonil,
sulfur,
other fungicides, such as acibenzolar-S-methyl, benthiavalicarb, carpropamid, chlorothalonil, cyflufenamid, cymoxanil, dazomet, diclomezine, diclocymet, diethofencarb, edifenphos, ethaboxam, fenhexamid, fentin acetate, fenoxanil, ferimzone, fluazinam, fosetyl, fosetyl-aluminum, phosphorous acid and its salts, iprovalicarb, hexachlorobenzene, mandipropamide, metrafenone, pencycuron, propamocarb, phthalide, tolclofos-methyl, quintozene or zoxamide, N-(2-(4-[3-(4-chlorophenyl)prop-2-ynyloxy]-3-methoxyphenyl)ethyl)-2-methanesulfonylamino-3-methylbutyramide, N-(2-(4-[3-(4-chlorophenyl)prop-2-ynyloxy]-3-methoxyphenyl)ethyl)-2-ethanesulfonylamino-3-methylbutyramide, 3-[5-(4-chlorophenyl)-2,3-dimethylisoxazolidin-3-yl]pyridine, methyl 3-(4-chlorophenyl)-3-(2-isopropoxycarbonylamino-3-methylbutyrylamino)propionate, 4-fluorophenyl N-(1-(1-(4-cyanophenyl)ethanesulfonyl)but-2-yl)carbamate,
strobilurins, such as azoxystrobin, dimoxystrobin, enestroburin, fluoxastrobin, kresoxim-methyl, metominostrobin, orysastrobin, picoxystrobin, pyraclostrobin or trifloxystrobin,
sulfenic acid derivatives, such as captafol, captan, dichlofluanid, folpet or tolylfluanid,
cinnamides and analogous compounds, such as dimethomorph, flumetover or flumorph.

### SYNTHESIS EXAMPLES

The procedures described in the synthesis examples below were used to prepare further compounds I by appropriate modification of the starting compounds. The compounds thus obtained are listed in the tables below, together with physical data.

### Example 1

### Preparation of 2-[5-chloro-6-(2,4,6-trifluorophenyl)-[1,2,4]triazolo[1,5-a]pyrimidin-7-ylamino]butan-1-ol

1.57 mmol of triethylamine and 1.57 mmol of 2-aminobutan-1-ol were added to a solution of 500 mg (1.57 mmol) of 5,7-dichloro-6-(2,4,6-trifluorophenyl)-[1,2,4]triazolo[1,5-a]pyrimidine in 4 ml of dichloromethane, and the solution was then stirred at 20 to 25° C. for about 15 hours. The solution was then extracted with 0.5 M HCl and 5% strength NaCl solution. The organic phases were dried and freed from the solvent. What remained were 535 mg of the title compound as light-yellow crystals of m.p. 76-77° C.

¹H-NMR (CDCl₃; δ in ppm): 8.2 (1H, s); 6.85 (2H, q); 6.7 (1H, broad, NH); 3.85 (1H, s, broad); 3.7 (2H, q); 3.5 (1H, s, broad); 1.65 (1H, m); 1.55 (1H, m); 0.8 (3H, t).

### Example 2

### Preparation of 2-[5-chloro-6-(2,4,6-trifluorophenyl)-[1,2,4]triazolo[1,5-a]pyrimidin-7-ylamino]butyl acetate

In each case 0.13 mmol of triethylamine, acetic anhydride and 4-N,N-dimethylaminopyridine were added to a solution of 50 mg (0.13 mmol) of 2-[5-chloro-6-(2,4,6-trifluorophenyl)-[1,2,4]triazolo[1,5-a]pyrimidin-7-ylamino]butan-1-ol (see Ex. 1) in 2 ml of dichloromethane. The solution was stirred at 20 to 25° C. for about 5 hours. The solution was then extracted with 0.5 M HCl and 5% strength NaCl solution. The organic phases were dried and freed from the solvent. What remained were 36 mg of the title compound as light-yellow crystals of m.p. 39-42° C.

¹H-NMR (CDCl₃; 6 in ppm): 8.4 (1H, s); 6.9 (2H, t); 6.4 (1H, broad, NH); 4.05 (2H, d); 3.7 (1H, broad); 2.0 (3H, s); 1.55 (2H, m); 0.85 (3H, t).

### Example 3

### Preparation of [5-chloro-6-(2,4,6-trifluorophenyl)-[1,2,4]triazolo[1,5-a]pyrimidin-7-yl]-(1-ethoxymethylpropyl)amine

0.05 mmol of caesium carbonate and 0.05 mmol of iodethane were added to a solution of 20 mg (0.05 mmol) of 2-[5-chloro-6-(2,4,6-trifluorophenyl)-[1,2,4]triazolo[1,5-a]pyrimidin-7-ylamino]butan-1-ol (see Ex. 1) in 1 ml of acetone, and the suspension was then stirred at 50° C. for 2 hours. After addition of 5 ml of dichloromethane, the mixture was extracted with 0.5 M HCl and 5% strength NaCl solution. The organic phases were dried and freed from the solvent. What remained were 9 mg of the title compound as a colorless oil.

¹H-NMR (CDCl₃; 6 in ppm): 8.35 (1H, s); 6.85 (2H, m); 6.6 (1H, broad, NH); 3.5 (1H, broad); 3.45 (2H, q); 3.4 (2H, d); 1.65 (1H, m); 1.5 (1H, m); 1.1 (3H, t); 0.75 (3H, t).

The HPLC retention times (RT) in the tables below were determined at 40° C. using the RP-18 column Chromolith Speed ROD (from Merck KgaA, Germany) using the mobile phase acetonitrile+0.1% trifluoroacetic acid (TFA)/water+0.1% TFA in a gradient from 5:95 to 95:5 over 5 min. Mass spectrometry was carried out using quadropole electrospray ionization, 80 V (positive mode).

TABLE I

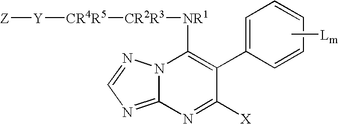

| | | | | | | | | | | phys. data HPLC |
|---|---|---|---|---|---|---|---|---|---|---|
| | | | | | | | | | | (RT [min]; MS [m/z]); |
| | | | | | | | | | | m.p. [° C.]; |
| No. | R¹ | R² | R³ | R⁴ | R⁵ | Y | Z | X | Lₘ | ¹H-NMR [δ ppm] |
| I-1 | H | CH₂CH₃ | H | H | H | O | CH₃ | Cl | 2,4,6-F₃ | 3.35 min, [386 M + H]⁺ |
| I-2 | H | CH₂CH₃ | H | H | H | O | CH₂CH₃ | Cl | 2,4,6-F₃ | 3.60 min; [400 M + H]⁺ |
| I-3 | H | CH₃ | H | H | H | O | CH₂CH₃ | Cl | 2,4,6-F₃ | 3.38 min; [385 M + H]⁺ |
| I-4 | H | CH₃ | H | H | H | O | C(CH₃)₃ | Cl | 2,4,6-F₃ | 3.85 min; [413 M + H]⁺ |
| I-5 | H | CH₃ | H | H | H | O | CH₂CH(CH₃)₂ | Cl | 2,4,6-F₃ | 3.90 min; [413 M + H]⁺ |
| I-6 | H | CH₃ | H | H | H | O | CH₂CH₂CH₃ | Cl | 2,4,6-F₃ | 3.65 min; [399 M + H]⁺ |
| I-7 | CH₃ | CH₂CH₃ | H | H | H | O | CH₃ | Cl | 2,4,6-F₃ | 3.36 min; [385 M + H]⁺ |
| I-8 | H | CH₂CH₃ | H | H | H | O | CH₂CH₃ | Cl | 2-Cl, 6-F | 3.56 min; [398 M + H]⁺ |
| I-9 | H | CH₂CH₃ | H | H | H | O | CH₂CH₃ | Cl | 2-Cl, 4-OCH₃ | 3.54 min; [410 M + H]⁺ |
| I-10 | H | CH(CH₃)₂ | H | H | H | O | H | Cl | 2,4,6-F₃ | 2.95 min; [385 M + H]⁺ |
| I-11 | H | CH₃ | H | CH₃ | H | O | H | Cl | 2,4,6-F₃ | 2.79 min; [371 M + H]⁺ |
| I-12 | H | C₆H₅ | H | H | H | O | H | Cl | 2,4,6-F₃ | 3.06 min; [419 M + H]⁺ |
| I-13 | H | CH₃ | H | H | H | O | H | Cl | 2,4,6-F₃ | 2.56 min; [357 M + H]⁺ |
| I-14 | H | C(CH₃)₃ | H | H | H | O | H | Cl | 2,4,6-F₃ | C*R²R³ in S config. |
| | | | | | | | | | | 166-168° C. |
| I-15 | H | CH₂CH₃ | H | H | H | O | H | Cl | 2,4,6-F₃ | 2.81 min; [371 M + H]⁺ |
| I-16 | H | CH₃ | H | CH₃ | CH₃ | O | H | Cl | 2,4,6-F₃ | 3.01 min; [385 M + H]⁺ |
| I-17 | H | CH₂CH₃ | H | H | H | O | C(O)CH₃ | Cl | 2,4,6-F₃ | 3.13 min; [413 M + H]⁺ |
| I-18 | H | CH₂CH₃ | H | H | H | O | C(O)CH₂CH₃ | Cl | 2,4,6-F₃ | 3.35 min; [427 M + H]⁺ |
| I-19 | H | CH₂CH₃ | H | H | H | O | C(O)CH(CH₃)₂ | Cl | 2.4.6-F₃ | 3.55 min; [441 M + H]⁺ |
| I-20 | H | CH(CH₃)₂ | H | H | H | O | C(O)CH₃ | Cl | 2,4,6-F₃ | 3.29 min; [427 M + H]⁺ |
| I-21 | H | CH(CH₃)₂ | H | H | H | O | C(O)CH₂CH₃ | Cl | 2,4,6-F₃ | 3.50 min; [441 M + H]⁺ |
| I-22 | H | CH(CH₃)₂ | H | H | H | O | C(O)CH(CH₃)₂ | Cl | 2,4,6-F₃ | 3.69 min; [455 M + H]⁺ |
| I-23 | H | CH₃ | H | CH₃ | H | O | C(O)CH₃ | Cl | 2,4,6-F₃ | 3.15 min; [413 M + H]⁺ |
| I-24 | H | CH₃ | H | CH₃ | H | O | C(O)CH₂CH₃ | Cl | 2,4,6-F₃ | 3.38 min; [427 M + H]⁺ |
| I-25 | H | CH₃ | H | CH₃ | H | O | C(O)CH(CH₃)₂ | Cl | 2,4,6-F₃ | 3.60 min; [441 M + H]⁺ |
| I-26 | H | C₆H₅ | H | H | H | O | C(O)CH₃ | Cl | 2,4,6-F₃ | 3.37 min; [461 M + H]⁺ |
| I-27 | H | C₆H₅ | H | H | H | O | C(O)CH₂CH₃ | Cl | 2,4,6-F₃ | 3.57 min; [475 M + H]⁺ |
| I-28 | H | C₆H₅ | H | H | H | O | C(O)CH(CH₃)₂ | Cl | 2,4,6-F₃ | 3.75 min; [489 M + H]⁺ |
| I-29 | H | CH₃ | H | H | H | O | C(O)CH₃ | Cl | 2,4,6-F₃ | 2.95 min; [399 M + H]⁺ |
| I-30 | H | CH₃ | H | H | H | O | C(O)CH₂CH₃ | Cl | 2,4,6-F₃ | 3.18 min; [413 M + H]⁺ |
| I-31 | H | CH₃ | H | H | H | O | C(O)CH(CH₃)₂ | Cl | 2,4,6-F₃ | 3.38 min; [427 M + H]⁺ |
| I-32 | H | CH₃ | H | C₆H₅ | H | O | H | Cl | 2,4,6-F₃ | 3.29 min; [433 M + H]⁺ |
| I-33 | H | CH₂CH₂CH₃ | H | H | H | O | H | Cl | 2,4,6-F₃ | 164-167° C. |
| I-34 | H | CH₂CH₂CH₃ | H | H | H | O | C(O)CH₃ | Cl | 2,4,6-F₃ | 3.34 min; [427 M + H]⁺ |
| I-35 | H | CH₂CH₂CH₃ | H | H | H | O | C(O)CH₂CH₃ | Cl | 2,4,6-F₃ | 3.54 min; [441 M + H]⁺ |
| I-36 | H | CH₂CH₂CH₃ | H | H | H | O | C(O)CH(CH₃)₂ | Cl | 2,4,6-F₃ | 3.72 min; [455 M + H]⁺ |
| I-37 | H | CH₂CH₂CH₃ | H | H | H | O | H | Cl | 2-Cl, 6-OCH₃ | 72-74° C. |
| I-38 | H | CH(CH₃)₂ | H | H | H | O | H | Cl | 2-Cl, 6-OCH₃ | 78-81° C. |
| I-39 | H | CH₃ | H | CH₃ | H | O | H | Cl | 2-Cl, 6-OCH₃ | 70-73° C. |
| I-40 | H | CH₃ | H | CH₃ | CH₃ | O | H | Cl | 2-Cl, 6-OCH₃ | 3.03 min; [395 M + H]⁺ |
| I-41 | H | CH₃ | H | H | H | O | H | Cl | 2-Cl, 6-OCH₃ | 2.67 min; [367 M + H]⁺ |
| I-42 | H | CH₂CH₃ | H | H | H | O | H | Cl | 2-Cl, 6-OCH₃ | 2.83 min; [381 M + H]⁺ |
| I-43 | H | CH₂CH₂CH₃ | H | H | H | O | H | Cl | 2-Cl, 6-F | 2.99 min; [383 M + H]⁺ |
| I-44 | H | CH(CH₃)₂ | H | H | H | O | H | Cl | 2-Cl, 6-F | 3.00 min; [383 M + H]⁺ |
| I-45 | H | CH₃ | H | CH₃ | H | O | H | Cl | 2-Cl, 6-F | 2.78 min; [369 M + H]⁺ |
| I-46 | H | CH₃ | H | CH₃ | CH₃ | O | H | Cl | 2-Cl, 6-F | 67-69° C. |
| I-47 | H | CH₃ | H | H | H | O | H | Cl | 2-Cl, 6-F | 76-79° C. |
| I-48 | H | CH₂CH₃ | H | H | H | O | H | Cl | 2-Cl, 6-F | 80-82° C. |
| I-49 | H | CH₃ | H | H | H | S | CH₂CH₂CH₃ | Cl | 2,4,6-F₃ | 3.72 min; [416 M + H]⁺ |
| I-50 | H | CH₂CH₃ | H | H | H | S | CH₂CH₃ | Cl | 2,4,6-F₃ | 3.67 min; [416 M + H]⁺ |
| I-51 | H | CH₃ | H | CH₃ | CH₃ | O | C(O)CH₃ | Cl | 2,4,6-F₃ | 3.45 min; [427 M + H]⁺ |
| I-52 | H | CH₃ | H | CH₃ | CH₃ | O | C(O)CH₂CH₃ | Cl | 2,4,6-F₃ | 3.70 min; [441 M + H]⁺ |
| I-53 | H | CH₃ | H | CH₃ | CH₃ | O | C(O)CH(CH₃)₂ | Cl | 2,4,6-F₃ | 3.92 min; [455 M + H]⁺ |
| I-54 | H | CH₃ | H | H | H | O | C(O)CHClCH₃ | Cl | 2,4,6-F₃ | 3.35 min; [447 M + H]⁺ |
| I-55 | H | CH(CH₃)₂ | H | H | H | O | C(O)CHClCH₃ | Cl | 2,4,6-F₃ | 3.65 min; [475 M + H]⁺ |
| I-56 | H | CH(CH₃)₂ | H | H | H | O | C(O)CHF₂ | Cl | 2,4,6-F₃ | 3.48 min; [463 M + H]⁺ |
| I-57 | H | CH(CH₃)₂ | H | H | H | O | C(O)CH₂CH(CH₃)₂ | Cl | 2,4,6-F₃ | 3.88 min; [469 M + H]⁺ |
| I-58 | H | CH(CH₃)₂ | H | H | H | O | C(O)C(═CH₂)CH₃ | Cl | 2,4,6-F₃ | 3.67 min; [453 M + H]⁺ |
| I-59 | H | CH₃ | H | H | H | O | C(O)CH₂CH(CH₃)₂ | Cl | 2,4,6-F₃ | 3.59 min; [441 M + H]⁺ |
| I-60 | H | CH₃ | H | H | H | O | C(O)C(═CH₂)CH₃ | Cl | 2,4,6-F₃ | 3.35 min; [425 M + H]⁺ |
| I-61 | H | CH₃ | H | CH₃ | CH₃ | O | C(O)CHClCH₃ | Cl | 2,4,6-F₃ | 3.74 min; [475 M + H]⁺ |
| I-62 | H | CH₃ | H | CH₃ | CH₃ | O | C(O)CHF₂ | Cl | 2,4,6-F₃ | 3.48 min; [463 M + H]⁺ |
| I-63 | H | CH₃ | H | CH₃ | CH₃ | O | C(O)CH₂CH(CH₃)₂ | Cl | 2,4,6-F₃ | 4.06 min; [469 M + H]⁺ |
| I-64 | H | CH₃ | H | CH₃ | CH₃ | O | C(O)C(═CH₂)CH₃ | Cl | 2,4,6-F₃ | 3.85 min; [453 M + H]⁺ |
| I-65 | H | C(CH₃)₃ | H | H | H | O | C(O)CH₃ | Cl | 2,4,6-F₃ | 3.51 min; [441 M + H]⁺ |
| I-66 | H | C(CH₃)₃ | H | H | H | O | C(O)CH₂CH₃ | Cl | 2,4,6-F₃ | 3.72 min; [455 M + H]⁺ |
| I-67 | H | C(CH₃)₃ | H | H | H | O | C(O)CH(CH₃)₂ | Cl | 2,4,6-F₃ | 3.90 min; [469 M + H]⁺ |
| I-68 | H | C(CH₃)₃ | H | H | H | O | C(O)CHClCH₃ | Cl | 2,4,6-F₃ | 3.83 min; [490 M + H]⁺ |
| I-69 | H | C(CH₃)₃ | H | H | H | O | C(O)CHF₂ | Cl | 2,4,6-F₃ | 3.66 min; [477 M + H]⁺ |
| I-70 | H | C(CH₃)₃ | H | H | H | O | C(O)CH₂CH(CH₃)₂ | Cl | 2,4,6-F₃ | 4.06 min; [483 M + H]⁺ |
| I-71 | H | CH(CH₃)₂ | H | H | H | O | H | Cl | 2,4,6-F₃ | 2.99 min; [385 M + H]⁺ |
| I-72 | H | CH₃ | CH₃ | H | H | S | CH₂CH₃ | Cl | 2,4,6-F₃ | 3.77 min; [415 M + H]⁺ |
| I-73 | H | CH₃ | CH₃ | H | H | S | CH₃ | Cl | 2,4,6-F₃ | 3.57 min; [401 M + H]⁺ |
| I-74 | H | CH₂CH₂CH₃ | H | H | H | O | C(O)CH₃ | Cl | 2-Cl, 4-OCH₃ | 3.39 min; [437 M + H]⁺ |
| I-75 | H | CH₂CH₂CH₃ | H | H | H | O | C(O)CH₂CH₃ | Cl | 2-Cl, 4-OCH₃ | 3.60 min; [452 M + H]⁺ |
| I-76 | H | CH₂CH₂CH₃ | H | H | H | O | C(O)CH(CH₃)₂ | Cl | 2-Cl, 4-OCH₃ | 3.80 min; [466 M + H]⁺ |
| I-77 | H | CH(CH₃)₂ | H | H | H | O | C(O)CH₃ | Cl | 2-Cl, 4-OCH₃ | 3.36 min; [438 M + H]⁺ |
| I-78 | H | CH(CH₃)₂ | H | H | H | O | C(O)CH₂CH₃ | Cl | 2-Cl, 4-OCH₃ | 3.57 min; [452 M + H]⁺ |
| I-79 | H | CH(CH₃)₂ | H | H | H | O | C(O)CH(CH₃)₂ | Cl | 2-Cl, 4-OCH₃ | 3.77 min; [466 M + H]⁺ |
| I-80 | H | CH₃ | H | CH₃ | H | O | C(O)CH₃ | Cl | 2-Cl, 4-OCH₃ | 3.24 min; [424 M + H]⁺ |
| I-81 | H | CH₃ | H | CH₃ | H | O | C(O)CH₂CH₃ | Cl | 2-Cl, 4-OCH₃ | 3.46 min; [438 M + H]⁺ |
| I-82 | H | CH₃ | H | CH₃ | H | O | C(O)CH(CH₃)₂ | Cl | 2-Cl, 4-OCH₃ | 3.65 min; [452 M + H]⁺ |
| I-83 | H | CH₃ | H | CH₃ | CH₃ | O | C(O)CH₃ | Cl | 2-Cl, 4-OCH₃ | 3.48 min; [438 M + H]⁺ |
| I-84 | H | CH₃ | H | CH₃ | CH₃ | O | C(O)CH₂CH₃ | Cl | 2-Cl, 4-OCH₃ | 3.71 min; [452 M + H]⁺ |
| I-85 | H | CH₃ | H | CH₃ | CH₃ | O | C(O)CH(CH₃)₂ | Cl | 2-Cl, 4-OCH₃ | 3.91 min; [466 M + H]⁺ |
| I-86 | H | CH₃ | H | H | H | O | C(O)CH₃ | Cl | 2-Cl, 4-OCH₃ | 3.02 min; [410 M + H]⁺ |
| I-87 | H | CH₃ | H | H | H | O | C(O)CH₂CH₃ | Cl | 2-Cl, 4-OCH₃ | 3.24 min; [424 M + H]⁺ |
| I-88 | H | CH₃ | H | H | H | O | C(O)CH(CH₃)₂ | Cl | 2-Cl, 4-OCH₃ | 3.45 min; [438 M + H]⁺ |
| I-89 | H | CH₂CH₃ | H | H | H | O | C(O)CH₃ | Cl | 2-Cl, 4-OCH₃ | 3.19 min; [424 M + H]⁺ |
| I-90 | H | CH₂CH₃ | H | H | H | O | C(O)CH₂CH₃ | Cl | 2-Cl, 4-OCH₃ | 3.42 min; [438 M + H]⁺ |
| I-91 | H | CH₂CH₃ | H | H | H | O | C(O)CH(CH₃)₂ | Cl | 2-Cl, 4-OCH₃ | 3.62 min; [452 M + H]⁺ |
| I-92 | H | CH₂CH₂CH₃ | H | H | H | O | C(O)CH₃ | Cl | 2-Cl, 6-F | 3.35 min; [426 M + H]⁺ |
| I-93 | H | CH₂CH₂CH₃ | H | H | H | O | C(O)CH₂CH₃ | Cl | 2-Cl, 6-F | 3.56 min; [440 M + H]⁺ |
| I-94 | H | CH₂CH₂CH₃ | H | H | H | O | C(O)CH(CH₃)₂ | Cl | 2-Cl, 6-F | 3.76 min; [454 M + H]⁺ |
| I-95 | H | CH(CH₃)₂ | H | H | H | O | C(O)CH₃ | Cl | 2-Cl, 6-F | 3.33 min; [426 M + H]⁺ |
| I-96 | H | CH(CH₃)₂ | H | H | H | O | C(O)CH₂CH₃ | Cl | 2-Cl, 6-F | 3.53 min; [440 M + H]⁺ |
| I-97 | H | CH(CH₃)₂ | H | H | H | O | C(O)CH(CH₃)₂ | Cl | 2-Cl, 6-F | 3.73 min; [454 M + H]⁺ |
| I-98 | H | CH₃ | H | CH₃ | H | O | C(O)CH₃ | Cl | 2-Cl, 6-F | 3.17 min; [412 M + H]⁺ |
| I-99 | H | CH₃ | H | CH₃ | H | O | C(O)CH₂CH₃ | Cl | 2-Cl, 6-F | 3.41 min; [426 M + H]⁺ |
| I-100 | H | CH₃ | H | CH₃ | H | O | C(O)CH(CH₃)₂ | Cl | 2-Cl, 6-F | 3.61 min; [440 M + H]⁺ |
| I-101 | H | CH₃ | CH₃ | CH₃ | H | O | C(O)CH₃ | Cl | 2-Cl, 6-F | 3.43 min; [426 M + H]⁺ |
| I-102 | H | CH₃ | CH₃ | CH₃ | H | O | C(O)CH₂CH₃ | Cl | 2-Cl, 6-F | 3.67 min; [440 M + H]⁺ |
| I-103 | H | CH₃ | CH₃ | CH₃ | H | O | C(O)CH(CH₃)₂ | Cl | 2-Cl, 6-F | 3.91 min; [454 M + H]⁺ |
| I-104 | H | CH₃ | H | H | H | O | C(O)CH₃ | Cl | 2-Cl, 6-F | 2.95 min; [398 M + H]⁺ |
| I-105 | H | CH₃ | H | H | H | O | C(O)CH₂CH₃ | Cl | 2-Cl, 6-F | 3.18 min; [412 M + H]⁺ |
| I-106 | H | CH₃ | H | H | H | O | C(O)OH(CH₃)₂ | Cl | 2-Cl, 6-F | 3.40 min; [426 M + H]⁺ |
| I-107 | H | CH₂CH₃ | H | H | H | O | C(O)CH₃ | Cl | 2-C1, 6-F | 3.14 min; [412 M + H]⁺ |
| I-108 | H | CH₂CH₃ | H | H | H | O | C(O)CH₂CH₃ | Cl | 2-Cl, 6-F | 3.36 min; [426 M + H]⁺ |
| I-109 | H | CH₂CH₃ | H | H | H | O | C(O)CH(CH₃)₂ | Cl | 2-Cl, 6-F | 3.57 min; [440 M + H]⁺ |
| I-110 | H | CH₂CH₃ | H | H | H | O | C(O)CHF₂ | Cl | 2,4,6-F₃ | 3.68 min; [421 M + H]⁺ |
| I-111 | H | C(CH₃)₃ | H | H | H | O | H | Cl | 2-Cl, 4-OCH₃ | 3.66 min; [409 M + H]⁺ |
| I-112 | H | C(CH₃)₃ | H | H | H | O | H | Cl | 2-Cl, 6-F | 3.29 min; [397 M + H]⁺ |
| I-113 | H | C(CH₃)₃ | H | H | H | O | C(O)CH₂CH₃ | Cl | 2-Cl, 4-OCH₃ | 3.76 min; [465 M + H]⁺ |
| I-114 | H | C(CH₃)₃ | H | H | H | O | C(O)CH(CH₃)₂ | Cl | 2-Cl, 4-OCH₃ | 3.93 min; [479 M + H]⁺ |
| I-115 | H | C(CH₃)₃ | H | H | H | O | C(O)CH₂CH₃ | Cl | 2-Cl, 6-F | 3.71 min; [453 M + H]⁺ |
| I-116 | H | C(CH₃)₃ | H | H | H | O | C(O)CH(CH₃)₂ | Cl | 2-C1, 6-F | 3.89 min; [467 M + H]⁺ |
| I-117 | H | CH₃ | H | H | H | O | CH₂CH₃ | Cl | 2,4,6-F₃ | 3.37 min; [386 M + H]⁺ |
| I-118 | H | CH₃ | H | H | H | O | C(O)C(CH₃)═CH₂ | Cl | 2,4,6-F₃ | 3.36 min; [425 M + H]⁺ |
| I-119 | H | CH₃ | H | CH₃ | CH₃ | O | C(O)CHClCH₃ | Cl | 2,4,6-F₃ | 3.74 min; [476 M + H]⁺ |
| I-120 | H | CH₃ | H | CH₃ | CH₃ | O | C(O)CHF₂ | Cl | 2,4,6-F₃ | 3.49 min; [463 M + H]⁺ |
| I-121 | H | CH₃ | H | CH₃ | CH₃ | O | C(O)CH₂CH(CH₃)₂ | Cl | 2,4,6-F₃ | 4.07 min; [469 M + H]⁺ |
| I-122 | H | CH₃ | H | CH₃ | CH₃ | O | C(O)C(CH₃)═CH₂ | Cl | 2,4,6-F₃ | 3.85 min; [453 M + H]⁺ |
| I-123 | H | C(CH₃)₃ | H | H | H | O | C(O)CH₃ | Cl | 2,4,6-F₃ | 3.52 min; [441 M + H]⁺ |
| I-124 | H | C(CH₃)₃ | H | H | H | O | C(O)CH₂CH₃ | Cl | 2,4,6-F₃ | 3.73 min; [455 M + H]⁺ |
| I-125 | H | C(CH₃)₃ | H | H | H | O | C(O)CH(CH₃)₂ | Cl | 2,4,6-F₃ | 3.91 min; [469 M + H]⁺ |
| I-126 | H | C(CH₃)₃ | H | H | H | O | C(O)CHClCH₃ | Cl | 2,4,6-F₃ | 3.84 min; [490 M + H]⁺ |
| I-127 | H | C(CH₃)₃ | H | H | H | O | C(O)CHF₂ | Cl | 2,4,6-F₃ | 3.67 min; [477 M + H]⁺ |
| I-128 | H | C(CH₃)₃ | H | H | H | O | C(O)CH₂CH(CH₃)₂ | Cl | 2,4,6-F₃ | 4.06 min; [483 M + H]⁺ |
| I-129 | H | C(CH₃)₃ | H | H | H | O | C(O)C(CH₃)═CH₂ | Cl | 2,4,6-F₃ | 3.87 min; [467 M + H]⁺ |
| I-130 | CH₃ | CH₃ | H | H | 3-methylisoxazolidin-5-yl | | | Cl | 2,4,6-F₃ | 3.36 min; [426 M + H]⁺ |
| I-131 | H | CH₃ | H | H | H | O | C₂H₅ | Cl | 2,4,6-F₃ | 3.36 min; [385 M + H]⁺ |
| I-132 | H | CH(CH₃)C₂H₅ | H | H | H | O | C(O)CH₃ | Cl | 2,4,6-F₃ | 3.50 min; [441 M + H]⁺ |
| I-133 | H | CH(CH₃)C₂H₅ | H | H | H | O | C(O)C₂H₅ | Cl | 2,4,6-F₃ | 3.70 min; [455 M + H]⁺ |
| I-134 | H | CH(CH₃)C₂H₅ | H | H | H | O | C(O)CH(CH₃)₂ | Cl | 2,4,6-F₃ | 3.88 min; [469 M + H]⁺ |
| I-135 | H | CH(CH₃)C₂H₅ | H | H | H | O | C(O)CHClCH₃ | Cl | 2,4,6-F₃ | 3.81 min; [489 M + H]⁺ |
| I-136 | H | CH(CH₃)C₂H₅ | H | H | H | O | C(O)CHF₂ | Cl | 2,4,6-F₃ | 3.64 min; [477 M + H]⁺ |
| I-137 | H | CH(CH₃)C₂H₅ | H | H | H | O | C(O)CH₂CH(CH₃)₂ | Cl | 2,4,6-F₃ | 4.04 min; [483 M + H]⁺ |
| I-138 | H | CH(CH₃)C₂H₅ | H | H | H | O | H | Cl | 2,4,6-F₃ | 3.18 min; [399 M + H]⁺ |
| I-139 | H | C(CH₃)₃ | H | H | H | O | H | OCH₃ | 2,4,6-F₃ | 3.08 min; [395 M + H]⁺ |
| I-140 | H | CH(CH₃)C₂H₅ | H | H | H | O | H | OCH₃ | 2,4,6-F₃ | 3.07 min; [395 M + H]⁺ |
| I-141 | H | CH(CH₃)C₂H₅ | H | H | H | O | H | Cl | 2-Cl, 6-F | 3.22 min; [398 M + H]⁺ |
| I-142 | H | CH(CH₃)C₂H₅ | H | H | H | O | H | Cl | 2-Cl, 4-OCH₃ | 3.25 min; [410 M + H]⁺ |
| I-143 | H | C(CH₃)₃ | H | ═O | | O | CH₃ | Cl | 2,4,6-F₃ | 3.56 min; [427 M + H]⁺ |
| I-144 | H | CH₃ | CH₃ | H | H | O | H | Cl | 2,4,6-F₃ | 1.4(6H); 3.4(2H); 5.1 |
| | | | | | | | | | | (1H); 6.5(1H); 6.9(2H); |
| | | | | | | | | | | 8.35 (1H) |
| I-145 | H | CH₃ | CH₃ | H | H | O | H | CH₃ | 2,4,6-F₃ | 149-153° C. |
| I-146 | H | CF₃ | H | ═O | | O | H | Cl | 2,4,6-F₃ | 2.85 min; [425 M + H]⁺ |
| I-147 | H | C(CH₃)₃ | H | H | H | O | C(O)OCH₃ | Cl | 2,4,6-F₃ | 3.30 min; [457 M + H]⁺ |
| I-148 | H | C(CH₃)₃ | H | H | H | O | C(O)N(CH₃)₂ | Cl | 2,4,6-F₃ | 3.27 min; [470 M + H]⁺ |
| I-149 | H | C(CH₃)₃ | H | H | H | O | SO₂CH₃ | Cl | 2,4,6-F₃ | 3.22 min; [477 M + H]⁺ |
| I-150 | H | —CH₂CH₂— | | H | H | O | H | Cl | 2,4,6-F₃ | 205-208° C. |
| I-151 | H | —CH₂CH₂— | | H | H | O | H | CH₃ | 2,4,6-F₃ | 197-200° C. |
| I-152 | H | C(CH₃)₃ | H | H | H | O | C(O)O(CH₂)₃CH₃ | Cl | 2,4,6-F₃ | 3.91 min; [499 M + H]⁺ |
| I-153 | H | C(CH₃)₃ | H | H | H | O | C(O)OCH₂CCl₃ | Cl | 2,4,6-F₃ | 3.94 min; [574 M + H]⁺ |
| I-154 | H | C(CH₃)₃ | H | H | H | O | C(O)O(CH₂)₂CH₃ | Cl | 2,4,6-F₃ | 3.72 min; [485 M + H]⁺ |
| I-155 | H | C(CH₃)₃ | H | H | H | O | 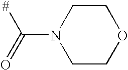 | Cl | 2,4,6-F₃ | 3.23 min; [512 M + H]⁺ |
| I-156 | H | C(CH₃)₃ | H | H | H | O | C(O)OCH₂CH₃ | Cl | 2,4,6-F₃ | 3.54 min; [471 M + H]⁺ |
| I-157 | H | C(CH₃)₃ | H | H | H | O | 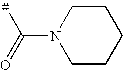 | Cl | 2,4,6-F₃ | 3.70 min; [510 M + H]⁺ |
| I-158 | H | C(CH₃)₃ | H | H | H | O | C(O)N[CH(CH₃)C₂H₅]₂ | Cl | 2,4,6-F₃ | 4.26 min; [555 M + H]⁺ |

# denotes the bond to Y

TABLE II

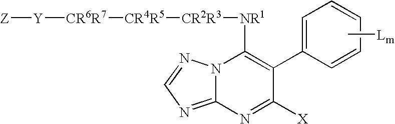

| | | | | | | | | | | | | phys. data HPLC (RT [min]; |
|---|---|---|---|---|---|---|---|---|---|---|---|---|
| No. | R¹ | R² | R³ | R⁴ | R⁵ | R⁶ | R⁷ | Y | Z | X | Lₘ | MS [m/z]); m.p. [° C.] |
| II-1 | H | CH₃ | H | H | H | H | H | O | H | Cl | 2,4,6-F₃ | 2.59 min; [371 M + H]⁺ |
| II-2 | H | CH₃ | H | H | H | H | H | O | C(O)CH₃ | Cl | 2,4,6-F₃ | 3.04 min; [413 M + H]⁺ |
| II-3 | H | CH₃ | H | H | H | H | H | O | C(O)CH₂CH₃ | Cl | 2,4,6-F₃ | 3.07 min; [427 M + H]⁺ |
| II-4 | H | CH₃ | H | H | H | H | H | O | C(O)CH(CH₃)₂ | Cl | 2,4,6-F₃ | 3.45 min; [441 M + H]⁺ |

### Examples of the Action Against Harmful Fungi

The fungicidal action of the compounds of the formula I was demonstrated by the following experiments:

The active compounds were formulated separately as a stock solution with 0.25% by weight of active compound in acetone or DMSO. 1% by weight of the emulsifier Wettol EM 31 (wetting agent having emulsifying and dispersing action based on ethoxylated alkylphenols) was added to this solution. The stock solutions of the active compounds were diluted with water to the stated concentration.

### Use Example 1

### Activity Against Early Blight of Tomatoes Caused by Alternaria solani

Leaves of potted plants of the cultivar “Golden Queen” were sprayed to run-off point with an aqueous suspension having the concentration of active compound stated below. The next day, the leaves were infected with an aqueous spore suspension of Alternaria solani in a 2% biomalt solution having a density of 0.17×10⁶ spores/ml. The plants were then placed in a water-vapor-saturated chamber at temperatures between 20 and 22° C. After 5 days, the disease on the untreated, but infected control plants had developed to such an extent that the infection could be determined visually in %.

In this test, the plants which had in each case been treated with 250 ppm of the active compounds Nos. I-1 to I-7, I-9, I-14, I-16, I-20, I-21, I-22, I-25, I-29, I-33 to I-40, I-43 to I-46, I-55, I-56, I-57, I-65, I-66, I-73 to I-80, I-83, I-84 and I-95 to I-97 had an infection level of not more than 15%, whereas the untreated plants were 90% infected.

### Use Example 2

### Activity Against Net Blotch of Barley Caused by Pyrenophora teres, 1 Day Protective Application

Leaves of potted barley seedlings of the cultivar “Hanna” were sprayed to run-off point with an aqueous suspension having the concentration of active compounds stated below. 24 hours after the spray coating had dried on, the test plants were inoculated with an aqueous spore suspension of Pyrenophora [syn. Drechslera] teres, the net blotch pathogen. The test plants were then placed in the greenhouse at temperatures between 20 and 24° C. and 95 to 100% relative atmospheric humidity. After 6 days, the extent of the development of the disease was determined visually in % infection of the entire leaf area.

In this test, the plants which had in each case been treated with 250 ppm of the active compounds Nos. I-1, I-2, I-6 to I-11, I-14 to I-24, I-33 to I-40, I-44, I-45, I-55 to I-5, I-71, I-74, I-76 and I-77 showed an infection level of not more than 10%, whereas the untreated plants were 90% infected.

1-14. (canceled)

15. A triazolopyrimidine of the formula I 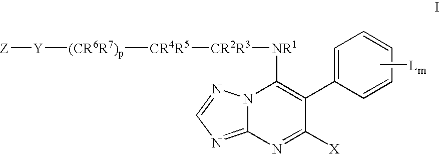
in which the substituents are as defined below:
R¹ is hydrogen, C₁-C₁₂-alkyl, C₁-C₁₂-haloalkyl, C₃-C₆-cycloalkyl, C₃-C₈-halocycloalkyl, C₂-C₁₂-alkenyl, C₂-C₁₂-haloalkenyl, C₃-C₆-cycloalkenyl, C₃-C₆-halocycloalkenyl, C₂-C₁₂-alkynyl, C₂-C₁₂-haloalkynyl or phenyl, naphthyl, or a five- or six-membered saturated, partially unsaturated or aromatic heterocycle, which comprises one to four heteroatoms from the group consisting of O, N and S,
R² is C₁-C₈-alkyl, C₁-C₆-haloalkyl, C₃-C₆-cycloalkyl, C₃-C₆-halocycloalkyl, C₂-C₈-alkenyl, C₂-C₈-haloalkenyl, C₃-C₆-cycloalkenyl, C₃-C₆-halocycloalkenyl, C₂-C₈-alkynyl, C₂-C₈-haloalkynyl or phenyl, naphthyl, or a five- or six-membered saturated, partially unsaturated or aromatic heterocycle which comprises one to four heteroatoms from the group consisting of O, N and S,
R³,R⁴,R⁵,R⁶,R⁷ are hydrogen or one of the groups mentioned under R²,
R⁴, together with R³ or R⁶, may also form a five- or six-membered saturated or unsaturated ring which, in addition to carbon atoms, may comprise one to three further heteroatoms from the group consisting of O, N and S as ring members and/or may carry one or more substituents R^{a};
R² with R³, R⁴ with R⁵, R⁶ with R⁷ may in each case together, with formation of carbonyl groups, also be oxygen and, with formation of spiro groups, form a C₂-C₅₋alkylene, or alkenylene or alkynylene chain which may be interrupted by one to three heteroatoms from the group consisting of O, N and S;
R¹ to R⁷ may in each case carry one to four identical or different groups R^{a}:
R^{a} is halogen, cyano, nitro, hydroxyl, C₁-C₆-alkyl, C₁-C₆-haloalkyl, C₁-C₆-alkylcarbonyl, C₃-C₆-cycloalkyl, C₁-C₆-alkoxy, C₁-C₆-haloalkoxy, C₁-C₆-alkoxycarbonyl, C₁-C₆-alkylthio, C₁-C₆-alkylamino, di-C₁-C₆-alkyl-amino, C₂-C₈-alkenyl, C₂-C₈-haloalkenyl, C₃-C₈-cycloalkenyl, C₂-C₆-alkenyloxy, C₃-C₆-haloalkenyloxy, C₂-C₆-alkynyl, C₂-C₆-haloalkynyl, C₃-C₆-alkynyloxy, C₃-C₆-haloalkynyloxy, C₃-C₆-cycloalkoxy, C₃-C₆-cycloalkenyloxy, oxy-C₁-C₃-alkyleneoxy, phenyl, naphthyl, a five- to ten-membered saturated, partially unsaturated or aromatic heterocycle which comprises one to four heteroatoms from the group consisting of O, N and S,
where these aliphatic, alicylic or aromatic groups for their part may be partially or fully halogenated or may carry one to three groups R^{b}:
R^{b} is halogen, cyano, nitro, hydroxyl, mercapto, amino, carboxyl, aminocarbonyl, aminothiocarbonyl, alkyl, haloalkyl, alkenyl, alkenyloxy, alkynyloxy, alkoxy, haloalkoxy, alkylthio, alkylamino, dialkylamino, formyl, alkylcarbonyl, alkylsulfonyl, alkylsulfoxyl, alkoxycarbonyl, alkylcarbonyloxy, alkylaminocarbonyl, dialkylaminocarbonyl, alkylaminothiocarbonyl, dialkylaminothiocarbonyl, where the alkyl groups in these radicals comprise 1 to 6 carbon atoms and the abovementioned alkenyl or alkynyl groups in these radicals comprise 2 to 8 carbon atoms;
and/or one to three of the following radicals:
cycloalkyl, cycloalkoxy, heterocyclyl, heterocyclyloxy, where the cyclic systems comprise 3 to 10 ring members; aryl,
aryloxy, arylthio, aryl-C₁-C₆-alkoxy, aryl-C₁-C₆-alkyl, hetaryl, hetaryloxy, hetarylthio, where the aryl radicals preferably comprise 6 to 10 ring members and the hetaryl radicals 5 or 6 ring members, where the cyclic systems may be partially or fully halogenated or substituted by alkyl or haloalkyl groups;
p is zero or 1;
L is halogen, C₁-C₄-alkyl, C₁-C₂-haloalkyl, C₁-C₄-alkoxy, cyano, nitro, amino, C₁-C₄-alkylamino, di-(C₁-C₄)-alkylamino, C₁-C₄-alkylcarbonylamino, C(O)—R, S(O)ₙ—R;
R is hydrogen, C₁-C₄-alkyl, C₁-C₂-haloalkyl, C₁-C₄-alkoxy, C₂-C₄-alkenyloxy, C₂-C₄-alkynyloxy, amino, C₁-C₄-alkylamino, di-(C₁-C₄)-alkylamino; where the groups R may be substituted by one to three groups R^{b};
n is zero, 1 or 2;
m is 1, 2, 3, 4 or 5;
X is halogen, cyano, C₁-C₄-alkyl, C₁-C₄-alkoxy, C₂-C₄-alkenyloxy, C₂-C₄-alkynyloxy or C₁-C₂-haloalkoxy,
Y is oxygen or sulfur;
Z is hydrogen, C₁-C₆-haloalkyl, C₃-C₆-cycloalkyl, C₁-C₈-alkylcarbonyl, C₁-C₈-alkoxycarbonyl, (C═O)NR^{A}R^{B}, C₃-C₈-alkenyloxycarbonyl, C₃-C₈-alkynyloxycarbonyl, C₃-C₆-cycloalkylcarbonyl, C₂-C₈-alkenyl, C₂-C₈-haloalkenyl, C₃-C₈-cycloalkenyl, C₂-C₆-alkynyl, C₂-C₆-haloalkynyl, C₃-C₆-cycloalkoxycarbonyl, C₃-C₆-cycloalkenyloxycarbonyl, C₁-C₈-alkylsulfinyl, C₁-C₈-alkylthio, C₁-C₈-alkylsulfonyl, phenyl, naphthyl, a five- to ten-membered saturated, partially unsaturated or aromatic heterocycle which comprises one to four heteroatoms from the group consisting of O, N and S; or
Z together with R⁵ or R⁷ may also form a five- or six-membered saturated or unsaturated ring which, in addition to carbon atoms and Y, may comprise one or two further heteroatoms from the group consisting of O, N and S as ring members and/or may carry one or more substituents R^{a};
the group Z may be partially or fully halogenated or carry one to three groups R^{b};
R^{A}, R^{B} are independently hydrogen, C₁-C₈-alkyl, C₂-C₈-alkenyl, C₂-C₈-alkynyl, C₃-C₆-cycloalkyl or C₃-C₆-cycloalkenyl;
R^{A} and R^{B} may combine with the nitrogen atom to which they are attached to form a five- or six-membered saturated, partially unsaturated or aromatic ring which, as well as carbon atoms, may comprise from one to three further heteroatoms from the group consisting of O, N and S as a ring member and/or may carry one or more substituents R^{a}.

16. The compound of the formula I according to claim 15 in which the substituents are as defined below:
R¹ is hydrogen, C₁-C₁₂-alkyl, C₁-C₁₂-haloalkyl, C₃-C₆-cycloalkyl, C₃-C₈-halocycloalkyl, C₂-C₁₂-alkenyl, C₂-C₁₂-haloalkenyl, C₃-C₆-cycloalkenyl, C₃-C₆-halocycloalkenyl, C₂-C₁₂-alkynyl, C₂-C₁₂-haloalkynyl or phenyl, naphthyl, or a five- or six-membered saturated, partially unsaturated or aromatic heterocycle which comprises one to four heteroatoms from the group consisting of O, N and S,
R² is C₁-C₈-alkyl, C₁-C₆-haloalkyl, C₃-C₆-cycloalkyl, C₃-C₆-halocycloalkyl, C₂-C₈-alkenyl, C₂-C₈-haloalkenyl, C₃-C₆-cycloalkenyl, C₃-C₆-halocycloalkenyl, C₂-C₈-alkynyl, C₂-C₈-haloalkynyl or phenyl, naphthyl, or a five- or six-membered saturated, partially unsaturated or aromatic heterocycle which comprises one to four heteroatoms from the group consisting of O, N and S,
R³,R⁴,R⁵,R⁶,R⁷ are hydrogen or one of the groups mentioned under R²,
R⁴, together with R³ or R⁶, may also form a five- or six-membered saturated or unsaturated ring which, in addition to carbon atoms, may comprise one to three further heteroatoms from the group consisting of O, N and S as ring members and/or may carry one or more substituents R^{a};
R² with R³, R⁴ with R⁵, R⁶ with R⁷ may in each case together, with formation of spiro groups, form a C₂-C₅-alkylene, or alkenylene or alkynylene chain which may be interrupted by one to three heteroatoms from the group consisting of O, N and S;
R¹ to R⁷ may in each case carry one to four identical or different groups R^{a} according to claim 1;
Z is hydrogen, C₁-C₆-haloalkyl, C₃-C₆-cycloalkyl, C₁-C₈-alkylcarbonyl, C₃-C₆-cycloalkylcarbonyl, C₂-C₈-alkenyl, C₂-C₈-haloalkenyl, C₃-C₈-cycloalkenyl, C₂-C₆-alkynyl, C₂-C₆-haloalkynyl, phenyl, naphthyl, a five- to ten-membered saturated, partially unsaturated or aromatic heterocycle which comprises one to four heteroatoms from the group consisting of O, N and S; or
Z together with R⁴ or R⁶ may also form a five- or six-membered saturated or unsaturated ring which, in addition to carbon atoms and Y, may comprise one or two further heteroatoms from the group consisting of O, N and S as ring members and/or may carry one or more substituents R^{a};
the group Z may be partially or fully halogenated or carry one to three groups R^{b} according to claim 1.

17. The compound of the formula I according to claim 15 in which X is chlorine or methyl.

18. The compound of the formula I according to claim 15 in which X is chlorine.

19. The compound of the formula I according to claim 15 in which the index p is zero.

20. The compound of the formula I according to claim 15 which conforms to the formula I.3 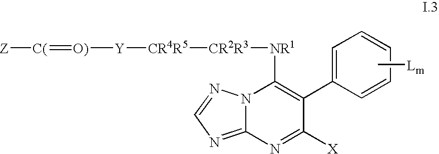
in which the variables are as defined according to the formula I and Z is defined as follows:
hydrogen, C₁-C₈-alkyl, C₁-C₈-alkoxy, C₃-C₆-cycloalkyl, C₃-C₈-alkenyloxy, C₃-C₈-alkynyloxy, C₃-C₆-cycloalkoxy, C₃-C₆-cycloalkenyloxy, NR^{A}R^{B}, a five- to ten-membered saturated, partially unsaturated or aromatic heterocycle which comprises one to four heteroatoms from the group consisting of O, N or S; the group Z may be partially or fully halogenated or carry one to three groups R^{b}.

21. The compound of the formula I according to claim 15 in which at least one group L is located in the ortho-position to the point of attachment to the triazolopyrimidine skeleton.

22. The compound of the formula I according to claim 15 in which the phenyl group substituted by Lₘ is the group A 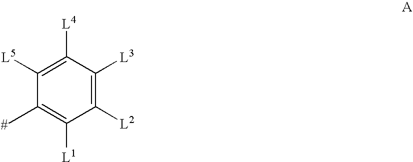
in which # is the point of attachment to the triazolopyrimidine skeleton and
L¹ is fluorine, chlorine, CH₃ or CF₃;
L²,L⁴ independently of one another are hydrogen or fluorine;
L³ is hydrogen, fluorine, chlorine, CN, CH₃, OCH₃, NH₂, NHCH₃, N(CH₃)₂, C(O)NH₂ or C(O)CH₃; and
L⁵ is hydrogen, fluorine, chlorine or CH₃.

23. The compound of the formula I according to claim 15 in which the phenyl group substituted by Lₘ is one of the combinations of substituents below: 2-fluoro-6-chloro, 2,6-difluoro, 2,6-dichloro, 2-fluoro-6-methyl, 2,4,6-trifluoro, 2,6-difluoro-4-methoxy, 2-chloro-4-methoxy, pentafluoro, 2-methyl-4-fluoro,
2-trifluoromethyl, 2-methoxy-6-fluoro, 2-chloro, 2-fluoro, 2,4-difluoro, 2-fluoro-4-chloro, 2-chloro-4-fluoro, 2-chloro-5-fluoro, 2,3-difluoro, 2,5-difluoro, 2,3,4-trifluoro, 2-methyl, 2,4-dimethyl, 2-methyl-4-chloro, 2-methyl-5-fluoro, 2-fluoro-4-methyl, 2,6-dimethyl, 2,4,6-trimethyl, 2,6-difluoro-4-methyl, 2-trifluoromethyl-4-fluoro, 2-trifluoromethyl-5-fluoro or 2-trifluoromethyl-5-chloro.

24. A process for preparing a compound of the formula I according to claim 15 in which X is halogen by reacting dihalotriazolopyrimidines of the formula II, 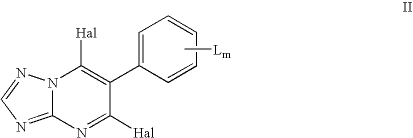
in which the variables are as defined for formula I and Hal is a halogen atom, with amines of the formula III

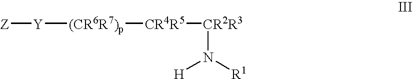

25. The process according to claim 24, wherein Hal is chlorine.

26. The process according to claim 24 in which Z is hydrogen.

27. A composition suitable for controlling harmful fungi, which composition comprises a solid or liquid carrier and a compound of the formula I according to claim 15.

28. The composition according to claim 27 which, as well as a compound of the formula I, additionally comprises a further active compound.

29. A method for controlling phytopathogenic harmful fungi, which method comprises treating the fungi or the materials, plants, the soil or seed to be protected against fungal attack with an effective amount of a compound of the formula I according to claim 15.

30. Seed comprising a compound of the formula I according to claim 15 in an amount from 1 to 1000 g per 100 kg.
